(19) United States
(12) Patent Application Publication
(10) Pub. No.: US 2026/0034277 A1
(43) Pub. Date: Feb. 5, 2026

(54) SYSTEMS AND METHODS FOR PATHOGEN INACTIVATION OF BLOOD PRODUCTS

(71) Applicant: Cerus Corporation, Concord, CA (US)

(72) Inventors: Peter BRINGMANN, Concord, CA (US); Daniel CHURCH, Danville, CA (US); Melody HOLTAN, Martinez, CA (US); Lloyd ISON, Livermore, CA (US); Naheed MUFTI, Danville, CA (US); Nils OLSSON, San Diego, CA (US); Veena PATA, Dublin, CA (US); Felicia SANTA MARIA, Bay Point, CA (US); Marc STERN, Oakland, CA (US); Blake WARBINGTON, Fairfield, CA (US)

(21) Appl. No.: 19/242,744

(22) Filed: Jun. 18, 2025

Related U.S. Application Data
(60) Provisional application No. 63/662,383, filed on Jun. 20, 2024.
(60) Provisional application No. 63/677,349, filed on Jul. 30, 2024.

(51) Int. Cl. A61M 1/02; A61L 2/00; G01N 5/00; G01N 33/49

(52) U.S. Cl. CPC A61M 1/0272; A61L 2/0047; A61L 2/0082; G01N 5/00; G01N 33/49; A61L 2202/11; A61L 2202/14; A61L 2202/22; A61M 2202/0427

(57) ABSTRACT

Provided are methods and systems for the pathogen inactivation of blood products and compositions, including methods of using devices (e.g., comprising component(s) configured to control flow of fluid(s)) to determine the amount of pathogen inactivation compound (PIC) necessary for pathogen inactivation of a blood composition (e.g., based at least in part on one or more input parameters) and allow transfer of the blood composition and the PIC into a container, e.g., of a processing set.

### CROSS-REFERENCE TO RELATED APPLICATIONS

This application claims the priority benefit of U.S. Provisional Application Nos. 63/662,383, filed Jun. 20, 2024, and 63/677,349, filed Jul. 30, 2024, the disclosures of each of which are incorporated herein by reference in their entirety.

### STATEMENT REGARDING FEDERALLY SPONSORED RESEARCH OR DEVELOPMENT

This invention was made with government support under Contract Nos. HHS0100201600009C and 75A50124C00046 awarded by the Department of Health and Human Services; Office of the Administration for Strategic Preparedness and Response (ASPR); Biomedical Advanced Research and Development Authority. The government has certain rights in the invention.

### TECHNICAL FIELD

The methods and systems described herein generally relate to the preparation of pathogen-inactivated blood compositions and products. More particularly the present disclosure relates to methods of using devices (e.g., comprising component(s) configured to control flow of fluid(s)) to determine the amount of pathogen inactivation compound (PIC) necessary for pathogen inactivation of a blood composition (e.g., based at least in part on one or more input parameters) and allow transfer of the blood composition and the PIC into a container, e.g., of a processing set.

### BACKGROUND

Blood component collection and processing serves a critical role in healthcare worldwide, and millions of units of donated blood components are collected by blood banks each year. While some units of whole blood are collected from donors and used directly for transfusion, most donations are generally separated into the blood components red blood cells, platelets and plasma for more specific therapeutic use. Separation may be either following collection of whole blood donations or at the point of collection if using a suitable separation device system, such as an apheresis collection device. Individual blood components are then used in treating different medical needs and conditions based on therapeutic need.

To minimize the risk of infecting an individual receiving blood or blood components, it is important to ensure that blood components are free of pathogens. Pathogen inactivation (PI) technologies increasingly are being used to treat blood components to help mitigate the risk of transfusion transmitted infection and complications arising from donor leukocytes. At least three PI technologies available commercially in certain countries utilize ultraviolet light, either in the presence or absence of a pathogen inactivation compound (e.g., photosensitizer), for treatment of one or more blood products, such as plasma, platelets, red blood cells and/or whole blood (see e.g., Irsch, 2011, Transf. Med. Hemother. 38:19-31; Prowse 2013, Vox Sang., 104,183-199; Schlenke, 2014, Transfus. Med. Hemother,. 41, 309-325). Such PI technologies include, for example, the amotosalen/UV based INTERCEPT® system (Cerus), the riboflavin/UV based Mirasol® system (TerumoBCT) and the UVC light only based Theraflex® system (Macopharma). At least one other PI technology in late-stage clinical development for treatment of red blood cells (RBCs) also targets nucleic acids, by utilizing frangible pathogen-inactivation compounds (e.g., S-303), but without the need for ultraviolet light (Henschler, 2011, Transfus. Med. Hemother., 38:33-42).

For plasma and platelet components, photochemical (e.g., photoactivated) compounds such as psoralens (e.g., amotosalen) with UVA may be used for pathogen inactivation (see e.g., Irsch, 2011; U.S. Pat. No. 5,593,823). Such inactivation methods may require specific guard band ranges for input platelet volumes and platelet numbers in order to achieve an effective compound concentration for pathogen inactivation. For example, a minimum concentration of compound is defined in order to achieve a certain level of pathogen inactivation, and a maximum concentration is defined to avoid impacting the function of the treated blood product. The psoralen-based photochemical inactivation technology utilizes a disposable processing set comprising a fixed volume of pathogen inactivation compound and a downstream compound adsorption device (CAD) for removing residual compound post-UVA.

For red blood cell-containing blood products, nucleic acid targeting compounds being developed for pathogen inactivation do not require photoactivation (see e.g., Henschler, 2011; U.S. Pat. Nos. 6,093,725 and 6,514,987), and one such compound, amustaline (S-303), has completed successful Phase 3 clinical testing. Such compounds (e.g., amustaline) for pathogen inactivation of RBCs include, for example, frangible nucleic acid targeted alkylators, comprising an acridine anchor (e.g., intercalator that targets nucleic acids), an effector (e.g., bis alkylator group that reacts with nucleophiles such as DNA bases) and a linker (e.g., small flexible carbon chain that contains a labile ester bond that can hydrolyze at neutral pH). Due to stability limitations of frangible compounds, the amustaline-based RBC pathogen inactivation technology utilizes a disposable processing set that includes the compound in a form that must be reconstituted shortly before use, together with a quencher, such as for example, glutathione (see e.g., U.S. Pat. No. 8,900,805), in a form that must also be reconstituted prior to use.

Due to the high demand for pathogen-inactivated blood components and the production thereof (including those that comprise platelets, plasma, or red blood cells), there remains a need for improved systems and methods for rapid and accurate dosing of pathogen inactivation compounds (such as those discussed supra) into blood components for pathogen inactivation.

### SUMMARY

The methods and systems described herein are useful for dosing of pathogen inactivation compounds into blood compositions in a rapid and accurate manner. These are thought to increase the speed and throughput of dosing a pathogen inactivation compound into a blood composition while maintaining accuracy of dosing and are amenable to a variety of pathogen inactivation compounds and blood compositions/products.

In one aspect, the present disclosure provides a method of preparing a pathogen inactivated blood composition, comprising: at a device comprising one or more components configured to control flow of one or more fluids: (a) coupling a container containing a pathogen inactivation compound (PIC container) to a first fluid path, optionally wherein the first fluid path is loaded to the device prior to coupling the PIC container to the first fluid path; (b) coupling (e.g., connecting) a processing set to the first fluid path; (c) transferring a blood composition from a container containing the blood composition (blood composition container) into a container of the processing set configured for mixing a blood composition with a PIC (mixing container); (d) determining an amount of a pathogen inactivation compound (PIC) to pathogen-inactivate the blood composition based at least in part on one or more input parameters; and (e) transferring the determined amount of the PIC from the PIC container into the mixing container via the first fluid path using the one or more components configured to control flow of fluids.

In some embodiments according to any one of the embodiments disclosed herein, the first fluid path is configured for multiple transfers (e.g., independent transfers) of PIC (e.g., to multiple processing sets). In some embodiments, the method further comprises, prior to (a): loading (e.g., mounting) the first fluid path to the device. In some embodiments, the mixing container is further configured for subjecting the blood composition in admixture with the PIC to ultraviolet light (e.g., illumination). In some embodiments, the amount of the PIC to pathogen-inactivate the blood composition is determined based at least in part on weight and/or volume of the blood composition. In some embodiments, the amount of the PIC to pathogen-inactivate the blood composition is determined based at least in part by calculating the volume of the blood composition based on the weight of the blood composition. In some embodiments, determining the amount of the PIC to pathogen-inactivate the blood composition further comprises weighing the blood composition in the blood composition container. In some embodiments, the device further comprises a scale (e.g., internal or external to a housing of the device), and the blood composition is weighed in the blood composition container using the scale to determine the weight of the blood composition (e.g., subtracting a tare weight of the container); wherein optionally the scale provides for motion (e.g., rocking, agitation) of the blood composition (e.g., when adding and/or mixing with PIC). In some embodiments, the device further comprises one or more components configured to receive an input, and determining the amount of the PIC to pathogen-inactivate the blood composition further comprises receiving an input indicating the weight of the blood composition. In some embodiments, the input further indicates the determined amount of the PIC. In some embodiments, the one or more components configured to receive an input comprise a scanner, optionally wherein the scanner is a barcode scanner, radio frequency identification (RFID) scanner, or quick response (QR) code scanner. In some embodiments, the one or more components configured to receive an input comprise a barcode scanner, and the received input comprises identifying information associated with the blood composition from the barcode scanner. In some embodiments, the one or more components configured to receive an input comprise a radio frequency identification (RFID) scanner, and the received input comprises identifying information associated with the blood composition from the RFID scanner. In some embodiments, the one or more components configured to receive an input comprise a quick response (QR) code scanner, and the received input comprises identifying information associated with the blood composition from the QR code scanner. In some embodiments, the device further comprises a scale, and the received input comprises data representing the weight of the blood composition from the scale. In some embodiments, the one or more components configured to receive an input comprise a touchscreen display or keyboard, and the received input is a user input from the touchscreen display or keyboard. In some embodiments, the one or more input parameters comprise one or more of: a type of the blood composition, a volume of the blood composition, a number of platelets or red blood cells in the blood composition, a type of solution (e.g., buffer, additive solution, plasma) in the blood composition, a type of PIC, a target PIC concentration, and a weight of the blood composition. In some embodiments, the one or more input parameters comprise one or more of: a type of the blood composition, a volume of the blood composition, a number of platelets or red blood cells in the blood composition, a type of solution in the blood composition, and a weight of the blood composition. In some embodiments, the device further comprises one or more processors, and the amount of the PIC to pathogen-inactivate the blood composition is determined using the one or more processors. In some embodiments, the methods further comprise reconstituting the PIC in a solution. In some embodiments, the methods further comprise reconstituting the PIC (e.g., from a powder, lyophilized solution, or otherwise dried form of PIC) in a solution. In some embodiments, the blood composition is transferred into the mixing container via a fluid path using gravity flow (e.g., a blood composition fluid path). In some embodiments, the one or more components configured to control flow of fluids comprise a pump. In some embodiments, the determined amount of the PIC is transferred into the mixing container via the first fluid path using the pump. In some embodiments, the blood composition is transferred into the mixing container via a fluid path (e.g., a blood composition fluid path) using the pump. In some embodiments, the blood composition is transferred into the mixing container prior to coupling the mixing container to the first fluid path or after coupling the mixing container to the first fluid path. In some embodiments, the device further comprises one or more supports configured to support the PIC container and/or the blood composition container. In some embodiments, the methods further comprise terminating fluid transfer from the PIC container into the mixing container via the first fluid path using one or more of the components configured to control flow of fluids. In some embodiments, the methods further comprise terminating fluid transfer from the PIC container into the mixing container via the first fluid path using one or more of the components configured to control flow of fluids based at least in part on detecting transfer of a weight (e.g., detecting an increased weight) corresponding to the determined amount of the PIC into the mixing container using the scale. In some embodiments, the methods further comprise terminating fluid transfer from the PIC container into the mixing container via the first fluid path using one or more of the components configured to control flow of fluids based at least in part on an input or signal from the pump or a time period after activation of the pump. In some embodiments, the methods further comprise terminating fluid transfer from the PIC container into the mixing container via the first fluid path using one or more of the components configured to control flow of fluids based at least in part on the pump transferring PIC for the amount of time required to transfer the determined amount of the PIC, e.g., as determined by flow rate. In some embodiments, the methods further comprise terminating fluid transfer from the PIC container into the mixing container via the first fluid path using one or more of the components configured to control flow of fluids based at least in part on detecting transfer of the determined amount of the PIC into the mixing container. In some embodiments, the device further comprises one or more fluid detectors configured to detect fluid transfer within the first fluid path. In some embodiments, the methods further comprise detecting transfer of the determined amount of the PIC into the mixing container via the first fluid path using the one or more fluid detectors. In some embodiments, the methods further comprise terminating fluid transfer from the PIC container into the mixing container via the first fluid path using one or more of the components configured to control flow of fluids based at least in part on detecting transfer of the determined amount of the PIC through the first fluid path using the one or more fluid detectors. In some embodiments, transfer of the determined amount of the PIC into the mixing container via the first fluid path is detected by measuring a volume of fluid transferred through the first fluid path using the one or more fluid detectors. In some embodiments, the device further comprises one or more fluid detectors configured to detect fluid transfer from the blood composition container via a fluid path (e.g., a blood composition fluid path) into the mixing container, and the method further comprises detecting transfer of the blood composition into the mixing container via the fluid path using the one or more fluid detectors. In some embodiments, the methods further comprise terminating fluid transfer from the blood composition container into the mixing container via the second fluid path using the one or more components configured to control flow of fluids based at least in part on detecting transfer of the blood composition through the second fluid path using the one or more fluid detectors. In some embodiments, the device further comprises a scale, and the method further comprises weighing the mixing container before, during, and/or after transfer of the determined amount of the PIC into the mixing container. In some embodiments, the methods further comprise terminating fluid transfer from the PIC container into the mixing container via the first fluid path using one or more of the components configured to control flow of fluids based at least in part on detecting transfer of a weight corresponding to the determined amount of the PIC into the mixing container using the scale. In some embodiments, the first fluid path comprises sterile tubing. In some embodiments, the first fluid path further comprises one or more of: a connector, a breakable connector, a cannula, a filter, a manifold, a pump, a container, and a coupling. In some embodiments, the first fluid path further comprises one or more of: a connector, a breakable connector, a cannula, a filter, a manifold, a container, and a coupling. In some embodiments, the methods further comprise coupling a container containing a diluent (diluent container) to the first fluid path (e.g., to a manifold of the first fluid path, to a fluid path connected to a manifold of the first fluid path), optionally wherein the diluent is a saline solution (saline). In some embodiments, the methods further comprise coupling a container containing a diluent (diluent container) to the first fluid path. In some embodiments, transferring the determined amount of the PIC from the PIC container into the mixing container via the first fluid path comprises: (i) transferring diluent from the diluent container into the PIC container (e.g., to yield a diluted concentration of PIC); and (ii) transferring an amount of the diluent solution and the PIC (e.g., diluted PIC) to provide the determined amount of the PIC from the PIC container into the mixing container. In some embodiments, transferring the determined amount of the PIC from the PIC container into the mixing container via the first fluid path comprises: (i) transferring PIC from the PIC container into the diluent container; and (ii) transferring an amount of the diluent solution and the PIC (e.g., diluted PIC) to provide the determined amount of the PIC from the diluent container into the mixing container. In some embodiments, transferring the determined amount of the PIC from the PIC container into the mixing container via the first fluid path comprises: (i) transferring diluent from the diluent container into a third container; (ii) transferring PIC from the PIC container into the third container; and (iii) transferring diluent and the determined amount of the PIC (e.g., diluted PIC) from the third container into the mixing container. In some embodiments, transferring the diluent and the PIC into the third container comprises transferring the diluent and the PIC via a manifold, wherein the third container is coupled to the manifold by a fluid path. In some embodiments, transferring the diluent and the PIC into the third container comprises transferring the diluent and the PIC via a manifold, wherein the third container is connected to the manifold by a fluid path. In some embodiments, the third container is coupled or configured to be coupled to the first fluid path. In some embodiments, the PIC in the PIC container is present at a concentration at least about 2 times greater than required to provide a solution comprising the diluent and the determined amount of the PIC (e.g., diluted PIC). In some embodiments, the PIC is diluted with the diluent at least about 2-fold. In some embodiments, the PIC in the PIC container is at a concentration at least about 2 times greater than the concentration of the determined amount of PIC (e.g., diluted PIC) transferred into the mixing container (e.g., than required to provide a solution comprising the diluent and the determined amount of the PIC). In some embodiments, the PIC in the PIC container is at a concentration about 2 times to about 50 times greater than the concentration of the determined amount of PIC (e.g., diluted PIC) transferred into the mixing container (e.g., than required to provide a solution comprising the diluent and the determined amount of the PIC). In some embodiments, the PIC in the PIC container is present at a concentration about 2 times to about 50 times, about 2 times to about 25 times, about 2 times to about 10 times about 5 times to about 25 times, about 5 times to about 15 times, or about 10 times to about 15 times greater than the concentration of the determined amount of PIC (e.g., diluted PIC) transferred into the mixing container (e.g., required to provide a solution comprising the diluent and the determined amount of the PIC). In some embodiments, the determined amount of the PIC is transferred into the mixing container at a volume accuracy of within about 5% of the determined amount. In some embodiments, the determined amount of the PIC is transferred into the mixing container at a volume accuracy of within about 2% of the determined amount. In some embodiments, amount of the diluent solution transferred into the PIC, the amount of PIC transferred into the diluent, and/or the amount of PIC and diluent transferred into the third container is transferred at a volume accuracy of within about 2% of a targeted volume. In some embodiments, amount of the diluent solution is transferred into the mixing container at a volume accuracy of within about 2% of a targeted volume. In some embodiments, the diluent comprises a saline solution (e.g., 0.9% NaCl) or water. In some embodiments, after the determined amount of the PIC is transferred into the mixing container via the first fluid path, less than about 0.3 mL of residual fluid is remaining in the first fluid path. In some embodiments, the methods further comprise after or concurrently with (e): admixing (e.g., mixing) the blood composition and the determined amount of the PIC in the mixing container. In some embodiments, the device is configured to agitate or rock the mixing container after or concurrently with transferring the determined amount of the PIC into the mixing container. In some embodiments, the device further comprises a scale, and the blood composition and the determined amount of the PIC are admixed in the mixing container on the scale. In some embodiments, the device is not an automated separation device (e.g., apheresis device). In some embodiments, the device is independent of an automated separation device (e.g., apheresis device). In some embodiments, the device is independent of an illumination device. In some embodiments, the processing set comprises: (i) the mixing container, wherein the mixing container is further configured for subjecting the blood composition in admixture with the PIC to illumination; and (ii) at least a first storage container, coupled to the container, wherein the storage container is configured for storing the pathogen-inactivated blood composition. In some embodiments, the processing set that comprises: (i) the container configured for mixing a blood composition with a PIC, wherein the container is further configured for subjecting the blood composition in admixture with the PIC to illumination; (ii) a compound adsorption device (CAD, container containing a CAD), coupled to the container, and (iii) at least a first storage container, coupled to the CAD, wherein the storage container is configured for storing the pathogen-inactivated blood composition. In some embodiments, the processing set comprises: (i) the mixing container, within which the blood composition in admixture with the determined amount of the PIC can be subjected to ultraviolet light (e.g., to photochemically inactivate a pathogen if present, wherein the mixing container is configured for subjecting the blood composition in admixture with the PIC to ultraviolet light, to photochemically inactivate pathogen(s) if present); and (ii) at least a first storage container, coupled to the mixing container, wherein the first storage container is configured for storing the pathogen-inactivated blood composition.

In some embodiments according to any one of the embodiments disclosed herein, the blood composition comprises a platelet and/or a plasma composition. In some embodiments, after (e) the PIC is at a concentration of about 40 μM and about 70 μM in the mixing container with the blood composition. In some embodiments, after (e) the PIC is at a concentration of about 40 μM and about 70 μM in the mixing container with the blood composition (e.g., after admixing with the blood composition). In some embodiments, after (e) the PIC is at a concentration of about 50 μM and about 60 μM, optionally about 55 μM, in the mixing container with the blood composition. In some embodiments, after (e) the PIC is at a concentration of about 50 μM and about 60 μM, optionally about 55 μM, in the mixing container with the blood composition (e.g., after admixing with the blood composition). In some embodiments, the one or more components configured to control flow of fluids comprise a pump, and wherein (e) comprises transferring the determined amount of the PIC from the PIC container into the mixing container via the first fluid path using the pump. In some embodiments, the mixing container is configured for photochemical inactivation of the blood composition using the determined amount of the PIC under sterile conditions. In some embodiments, the mixing container is configured for subjecting the blood composition in admixture with the PIC to ultraviolet light (e.g., to photochemically inactivate pathogen(s) if present in the blood composition) under sterile conditions. In some embodiments, the methods further comprise calculating a light dose for subjecting the blood composition and PIC to ultraviolet light to photochemically inactivate pathogen(s) if present in the blood composition using the determined amount of the PIC based at least in part on one or more of: a type of the blood composition, a volume of the blood composition, a type of PIC, a concentration of the PIC, a volume of the container for illumination, a number of platelets in the blood composition, a type of solution in the blood composition, and a weight of the blood composition. In some embodiments, the methods further comprise calculating a light dose for subjecting the blood composition and PIC to ultraviolet light to photochemically inactivate pathogen(s) if present in the blood composition using the determined amount of the PIC based at least in part on a volume of the blood composition and a concentration of the PIC, and optionally a volume of the container for illumination. In some embodiments, the methods further comprise transferring to a second device or server data to calculate a light dose for photochemical inactivation of the blood composition using the determined amount of the PIC. In some embodiments, the second device is an illumination device (e.g., configured to subject the blood composition and PIC to ultraviolet light). In some embodiments, the methods further comprise, after (e): subjecting the blood composition to illumination in the mixing container using the determined amount of the PIC (e.g., for photochemical inactivation of a pathogen, if present in the blood composition). In some embodiments, subjecting the blood composition to illumination comprises illuminating for a duration of between 1 second and 2 hours; and/or illuminating at an intensity between 1 and 1000 mW/cm²; and/or illuminating with a total dose of ultraviolet light between about 0.5 J/cm² to about 50 J/cm². In some embodiments, the methods further comprise, after subjecting the blood composition to illumination: transferring the pathogen-inactivated blood composition from the container into one or more storage containers. In some embodiments, the method does not comprise subjecting the pathogen-inactivated blood composition to a compound removal step, optionally wherein the method does not comprise contacting the pathogen-inactivated blood composition with a compound adsorption device (CAD). In some embodiments, the methods further comprise, after subjecting the blood composition to illumination: transferring the pathogen-inactivated blood composition from the container into a container containing a CAD. In some embodiments, the methods further comprise calculating a light dose for illumination of the blood composition using the determined amount of the PIC based at least in part on one or more of: a type of the blood composition, a volume of the blood composition, a number of platelets in the blood composition, a type of solution in the blood composition, and a weight of the blood composition. In some embodiments, the methods further comprise transferring data indicating the calculated light dose to a second device or server. In some embodiments, the PIC is a photoactive pathogen inactivating compound selected from the group consisting of a psoralen, an isoalloxazine, an alloxazine, a phthalocyanine, a phenothiazine, a porphyrin, and merocyanine 540. In some embodiments, the PIC is amotosalen. In some embodiments, the determined amount of the PIC is transferred into the mixing container at a volume accuracy of within about 1%, within about 2%, within about 3%, within about 4% or within about 5% of the determined amount. In some embodiments, the processing set comprises: (i) the mixing container, which is configured for mixing the blood composition and the determined amount of the PIC; and (ii) a second container, coupled to the container, wherein the second container is configured for incubating the blood composition and the determined amount of the PIC; wherein optionally the processing set comprises one or more filters (e.g., 0.2 μm filters). In some embodiments, the processing set comprises: (i) the mixing container, within which the blood composition in admixture with the determined amount of the PIC can be subjected to ultraviolet light; and (ii) at least a first storage container, coupled to the mixing container, wherein the first storage container is configured for storing the pathogen-inactivated blood composition; wherein optionally the processing set comprises one or more filters (e.g., 0.2 μm filters). In some embodiments, the processing set comprises: (i) the mixing container, which is configured for mixing the blood composition and the determined amount of the PIC; and (ii) a second container, coupled to the container, wherein the second container is configured for incubating the blood composition and the determined amount of the PIC. In some embodiments, the processing set further comprises (iii) at least a first storage container, coupled (e.g., by a fluid path) to the second container, wherein the first storage container is configured for storing the pathogen-inactivated blood composition. In some embodiments, the processing set comprises: (i) the container configured for mixing a blood composition with a PIC, wherein the container is further configured for subjecting the blood composition in admixture with the PIC to illumination; and (ii) at least a first storage container, coupled to the container, wherein the first storage container is configured for storing the pathogen-inactivated blood composition. In some embodiments, the processing set comprises: (i) the container configured for mixing the blood composition with the PIC; (ii) a second container, coupled to the container, wherein the second container is configured for incubating the blood composition and the determined amount of the PIC; and (iii) at least a first storage container, coupled to the second container, wherein the storage container is configured for storing the pathogen-inactivated blood composition.

In some embodiments according to any one of the embodiments disclosed herein, the blood composition comprises red blood cells (RBCs), e.g., a RBC composition. In some embodiments, the methods further comprise mixing the blood composition with a quencher. In some embodiments, mixing the blood composition with a quencher comprises: at a second device comprising one or more components configured to control flow of one or more fluids: (i) coupling a container containing a quencher (quencher container) to a first fluid path at the second device; (ii) coupling a processing set to the first fluid path at the second device; (iii) determining an amount of quencher to quench a pathogen inactivation compound (PIC) based at least in part on one or more input parameters; and (iv) transferring the determined amount of the quencher from the quencher container into a container of the processing set configured for mixing the blood composition with the quencher (mixing container) using one or more of the components configured to control flow of fluids of the second device; wherein the blood composition is transferred from the blood composition container into the mixing container in performance of step (c) before coupling the processing set of (b) to the first fluid path at the first device. In some embodiments, mixing the blood composition with a quencher comprises: at a second device comprising one or more components configured to control flow of one or more fluids: (i) coupling a container containing a quencher (quencher container) to a first fluid path; (ii) coupling a mixing container to the first fluid path; (iii) determining an amount of quencher to quench a pathogen inactivation compound (PIC) based at least in part on one or more input parameters; (iv) transferring the determined amount of the quencher from the quencher container into the mixing container using one or more of the components configured to control flow of fluids of the second device; and (v) transferring the blood composition from a container containing the blood composition (blood composition container) into the mixing container. In some embodiments, the quencher is added to the mixing container before the blood composition is added to the mixing container or after the blood composition is added to the mixing container. In some embodiments, the quencher and blood composition are mixed prior to mixing the PIC and blood composition. In some embodiments, the methods further comprise uncoupling the mixing container containing the blood composition and the quencher from the second device and coupling the uncoupled mixing container to the first device. In some embodiments, the methods further comprise: (i) coupling a quencher container to a second fluid path; and (ii) transferring quencher from the quencher container into the mixing container via the second fluid path using one or more of the components configured to control flow of fluids. In some embodiments, the quencher is added to the mixing container before the blood composition is added to the mixing container. In some embodiments, the quencher and blood composition are mixed prior to mixing the PIC and blood composition. In some embodiments, the one or more components configured to control flow of fluids comprise a manifold; wherein the PIC container is connected to the manifold via a first segment of the first fluid path; wherein the manifold is connected to the mixing container via a second segment of the first fluid path, and wherein the quencher container is connected to the manifold via the second fluid path; optionally, wherein the quencher container is connected to the manifold via a first segment of the second fluid path; wherein the manifold is further connected to the mixing container via a second segment of the second fluid path. In some embodiments, the second segment of the first fluid path comprises a Y-junction, and wherein the blood composition container is connected to the Y-junction. In some embodiments, the blood composition container is connected to a Y-junction as part of a processing set comprising the blood composition container (e.g., the Y-junction is part of a sterile tubing or fluid path of a processing set comprising the blood composition container and at least one other container). In some embodiments, the manifold is operably linked to an air filter. In some embodiments, the one or more components configured to control flow of fluids comprise a first manifold and a second manifold; wherein the PIC container is connected to the first manifold via a first segment of the first fluid path; wherein the manifold is connected to the processing set (e.g., mixing container) via a second segment of the first fluid path, and wherein the quencher container is connected to the second manifold via the second fluid path, optionally, wherein the quencher container is connected to the second manifold via a first segment of the second fluid path; and wherein the manifold is further connected to the processing set (e.g., mixing container) via a second segment of the second fluid path; optionally, the method further comprises coupling a processing solution container to the second fluid path and transferring the processing solution to the processing set using the second manifold. In some embodiments, the methods further comprise prior to transferring the quencher from the quencher container into the mixing container: reconstituting the quencher in a solution. In some embodiments, the PIC comprises a nucleic acid binding ligand that is an intercalator. In some embodiments, the intercalator is an acridine. In some embodiments, the PIC is β-alanine, N-(acridin-9-yl), 2-[bis(2-chloroethyl)amino]ethyl ester. In some embodiments, the quencher comprises cysteine or a derivative of cysteine. In some embodiments, the quencher is a peptide of 3-6 amino acids, wherein at least one of the amino acids is cysteine, N-acetyl cysteine, or S-acetyl cysteine. In some embodiments, the quencher is glutathione or a pharmaceutically acceptable salt thereof. In some embodiments, the quencher is glutathione monosodium salt. In some embodiments, the determined amount of the PIC is transferred into the mixing container at a volume accuracy of within about 2% of the determined amount. In some embodiments, the quencher is transferred into the mixing container at a volume accuracy of within about 1%, within about 2%, within about 3%, within about 4% or within about 5% of the determined amount. In some embodiments, the device is configured to agitate or rock the mixing container after or concurrently with transferring the determined amount of the quencher into the mixing container. In some embodiments, the mixing container is part of a processing set that comprises: (i) the mixing container, which is configured for incubating the blood composition and the determined amount of the PIC; (ii) a second container, coupled to the mixing container, wherein the second container is configured for incubating the blood composition and the determined amount of the PIC; and (iii) at least a first storage container, coupled to the second container, wherein the first storage container is configured for storing the pathogen-inactivated blood composition. In some embodiments, the mixing container contains a processing solution. In some embodiments, the storage container contains an additive solution. In some embodiments, the methods further comprise mixing the blood composition with a processing solution, optionally wherein mixing with processing solution with the blood composition occurs prior to mixing the quencher with the blood composition. In some embodiments, the mixing container contains a processing solution (e.g., treatment solution). In some embodiments, the methods further comprise transferring a processing solution from a processing solution container into the mixing container using one or more of the components configured to control flow of fluids at the second device. In some embodiments, the processing solution is added to the mixing container before the blood composition is added to the mixing container. In some embodiments, the mixing container contains a processing solution. In some embodiments, the methods further comprise transferring a processing solution from a processing solution container into the mixing container using one or more of the components configured to control flow of fluids at the second device. In some embodiments, the processing solution is added to the mixing container before the blood composition is added to the mixing container. In some embodiments, the processing solution comprises one or more of dextrose, adenine, mannitol, citrate, and citric acid. In some embodiments, the methods further comprise uncoupling the processing set from the first fluid path and the second fluid path. In some embodiments, the methods further comprise transferring the mixture of the blood composition, PIC, quencher and processing solution into the second container and incubating the mixture. In some embodiments, replacing the PIC, quencher and processing solution with an additive solution, comprising connecting a storage container containing the additive solution to the second container, separating the PIC, quencher and processing solution from the RBCs, and mixing the additive solution with the RBCs to yield pathogen inactivated RBC blood composition.

In some embodiments according to any one of the embodiments disclosed herein, the first fluid path is configured for multiple uses. In some embodiments, the PIC container, the processing set, and/or the mixing container are reversibly coupled to the first fluid path. In some embodiments, the quencher container is reversibly coupled to the first or the second fluid path. In some embodiments, the reversible coupling is a sterile, reversible coupling; optionally, wherein the sterile, reversible coupling is using a sterile connector. In some embodiments, the methods further comprise (e.g., prior to coupling a container to the first fluid path) affixing the first fluid path to the device. In some embodiments, the first fluid path is part of a reusable consumable configured for use in a dosing device system, wherein the reusable consumable comprises one or more fluid path(s), one or more connector(s), one or more manifold(s), and optionally one or more pump(s).

In another aspect, the present disclosure provides a system for preparing (e.g., for use in preparing) a pathogen inactivated blood composition, comprising: (a) a fluid path (e.g., comprising sterile tubing) configured to be removably mounted to the device, configured to be coupled to a container containing a pathogen inactivation compound (PIC), and configured to be coupled to a container of a processing set of the present disclosure; (b) a device comprising one or more components configured to control flow of fluids through the fluid path; and (c) the processing set. In some embodiments, the processing set comprises: (i) a container configured for mixing the blood composition with the PIC (mixing container), wherein the mixing container is configured to be coupled (e.g., reversibly coupled) to the fluid path; and (ii) at least a first storage container, coupled to the container, wherein the storage container is configured for storing the pathogen-inactivated blood composition. In some embodiments, the processing set comprises: (i) a container configured for mixing the blood composition with the PIC (mixing container), wherein the mixing container is configured to be coupled (e.g., reversibly coupled, sterile coupled) to the fluid path; (ii) a second container, coupled to the container, wherein the second container is configured for incubating a blood composition and the PIC; and (iii) at least a first storage container, coupled to the second container, wherein the first storage container is configured for storing the pathogen-inactivated blood composition. In some embodiments, the processing set comprises: (i) a mixing container for containing (e.g., mixing) a blood composition in admixture with the PIC (mixing container), wherein the mixing container is configured to be coupled (e.g., reversibly coupled, sterile coupled) to the fluid path; (ii) a second container, coupled to the mixing container, wherein the second container is configured for photochemical inactivation of a blood composition in admixture with the PIC; and (iii) at least a first storage container, coupled to the second container, wherein the first storage container is configured for storing the pathogen-inactivated blood composition. In some embodiments, the processing set comprises: (i) a container for containing (e.g., mixing) a blood composition in admixture with the PIC (mixing container), wherein the mixing container is configured to be coupled (e.g., reversibly coupled, sterile coupled) to the fluid path; (ii) a second container, coupled to the mixing container, wherein the second container contains a compound adsorption device (CAD); and (iii) at least a first storage container, coupled to the second container, wherein the first storage container is configured for storing the pathogen-inactivated blood composition.

In another aspect, the present disclosure provides a system for preparing a pathogen inactivated blood composition, comprising: (a) a device comprising one or more components configured to control flow of fluids through a fluid path, and (b) a processing set, wherein the processing set comprises a mixing container, the mixing container connected to a fluid path, wherein the fluid path comprises: (i) a first segment extending from the mixing container to a junction, (ii) a junction (e.g., Y-junction) connected to the first segment, (iii) a second segment connected to the junction, wherein the second segment is coupled or configured to be coupled to a container containing a PIC, and (iv) a third segment connected to the junction, wherein the third segment is configured to be coupled to a container containing a blood composition. In some embodiments the mixing container is configured for photochemical inactivation of a blood composition in admixture with a PIC (e.g., a determined amount of a PIC). In some embodiments, the second segment is coupled to a container containing a PIC. In some embodiments, each of the first, second and third segments of the fluid path comprise tubing. In some embodiments, the mixing container is connected to one or more storage containers, the storage container(s) configured to store a pathogen inactivated fluid. In some embodiments, the mixing container is connected to a container containing a compound adsorption device (CAD container). In some embodiments, the CAD container is connected to one or more storage containers, the storage container(s) configured to store a pathogen inactivated fluid.

In another aspect, the present disclosure provides a system for preparing a pathogen inactivated blood composition, comprising: (a) a fluid path (e.g., a first fluid path) configured to be removably mounted to a device for controlling flow of one or more fluids, wherein the fluid path is configured to be coupled to a container containing a pathogen inactivation compound (PIC), and configured to be coupled to a processing set (e.g., a mixing container of a processing set), and wherein the fluid path is configured for transfer of the PIC to the processing set; optionally wherein the fluid path is configured to be coupled to the container containing the PIC and to the processing set via one or more connectors; (b) a device comprising one or more components configured to control flow of one or more fluids through the fluid path; and (c) the processing set, comprising: (i) a container configured for mixing a blood composition with the PIC (mixing container), wherein the mixing container is configured to be coupled to the fluid path; and at least a first storage container, coupled to the mixing container, wherein the storage container is configured for storing the pathogen-inactivated blood composition; or (ii) a container configured for mixing a blood composition with a PIC (mixing container), wherein the mixing container is configured to be coupled to the fluid path; a second container, coupled to the mixing container, wherein the second container is configured for incubating the blood composition in admixture with the PIC and the second container is configured to be connected to at least a first storage container configured for storing the pathogen-inactivated blood composition; or (iii) a container configured for mixing a blood composition with a PIC (mixing container), wherein the mixing container is configured to be coupled to the fluid path; a second container, coupled to the mixing container, wherein the second container is configured for incubating the blood composition and the PIC; and at least a first storage container, coupled to the second container, wherein the storage container is configured for storing the pathogen-inactivated blood composition; or (iv) a container configured for mixing a blood composition with a PIC (mixing container), wherein the mixing container is configured to be coupled to the fluid path; a second container, coupled to the mixing container, wherein the second container contains (or comprises) a compound adsorption device (CAD); and at least a first storage container, coupled to the second container, wherein the storage container is configured for storing the pathogen-inactivated blood composition.

In some embodiments according to any one of the aspects and/or embodiments disclosed herein, the first fluid path is configured for multiple transfers (e.g., independent transfers, sequential transfers) of PIC (e.g., to multiple processing sets). In some embodiments, the first fluid path is part of a reusable consumable of the system, wherein the reusable consumable comprises one or more fluid paths, one or more connectors, one or more manifolds, and optionally one or more pumps; optionally wherein the first fluid path comprises a manifold and/or a pump. In some embodiments, the mixing container is configured for subjecting a blood composition and PIC contained therein to ultraviolet light (e.g., illumination). In some embodiments, the systems further comprise a second fluid path configured to be removably mounted to a device for controlling flow of one or more fluids, wherein the second fluid path is configured to be coupled to a container containing a quencher, and configured to be coupled to the processing set, and wherein the second fluid path is configured for transfer of quencher to the processing set; optionally, wherein the second fluid path is part of a reusable consumable of the system, wherein the reusable consumable comprises one or more fluid paths, one or more connectors, one or more manifolds, and optionally one or more pumps; optionally, wherein the second fluid path comprises a manifold and/or a pump. In some embodiments, the systems further comprise a second fluid path configured to be removably mounted to a device for controlling flow of one or more fluids, wherein the second fluid path is configured to be coupled to a container containing a quencher, and configured to be coupled to the processing set (e.g., mixing container of the processing set), and wherein the second fluid path is configured fo a third fluid path configured to be removably mounted to a device for controlling flow of one or more fluids, wherein the third fluid path is configured to be coupled to a container containing a processing solution, and wherein the third fluid path is connected to a manifold (e.g., manifold of the second fluid path) or configured to be coupled to the processing set, and wherein the third fluid path is configured for transfer of processing solution to the processing set. In some embodiments, the systems further comprise a fourth fluid path configured to be removably mounted to a device for controlling flow of one or more fluids, wherein the fourth fluid path is configured to be coupled to a container containing a diluent solution, and wherein the fourth fluid path is connected to a manifold (e.g., manifold of the first fluid path), and wherein the manifold is configured for mixing the PIC and the diluent. In some embodiments, the manifold is configured for mixing the PIC and the diluent in a container (dilution container) connected to the manifold by a fluid path. In some embodiments, the device further comprises one or more components configured to receive an input (e.g., an input indicating the weight of the blood composition and/or determined amount of the PIC); optionally, wherein the input comprises one or more of a type of the blood composition, a volume of the blood composition, a number of platelets or red blood cells in the blood composition, a type of solution (e.g., buffer, additive solution, plasma) in the blood composition, a type of PIC, a target concentration of PIC, and a weight of the blood composition. In some embodiments, the one or more components configured to receive an input comprise one or more of a scanner, barcode scanner, radio frequency identification (RFID) scanner, quick response (QR) code scanner, touchscreen display, or keyboard. In some embodiments, the one or more components configured to receive an input comprise one or more of a barcode scanner, radio frequency identification (RFID) scanner, quick response (QR) code scanner, touchscreen display, or keyboard. In some embodiments, the device further comprises a scale (e.g., internal or external to a housing of the device) configured to weigh a blood composition and/or the mixing container. In some embodiments, the device further comprises one or more processors configured to determine an amount of the PIC to add to the blood composition to pathogen-inactivate the blood composition; optionally, wherein the amount of the PIC is determined based at least in part on weight and/or volume of the blood composition; optionally, wherein the amount of the PIC is determined based at least in part by calculating the volume of the blood composition based on the weight of the blood composition. In some embodiments, the device further comprises a scale configured to weigh a blood composition and/or the mixing container. In some embodiments, the device further comprises one or more processors configured to determine an amount of the quencher to add to the blood composition to pathogen inactivate the blood composition. In some embodiments, the device further comprises one or more processors configured to determine an amount of the quencher to add to the blood composition to pathogen inactivate the blood composition; optionally, wherein the amount of the quencher is determined based at least in part on weight and/or volume of the blood composition; optionally, wherein the amount of the quencher is determined based at least in part by calculating the volume of the blood composition based on the weight of the blood composition. In some embodiments, the device is configured to calculate a light dose for subjecting the blood composition and PIC to ultraviolet light to photochemically inactivate pathogen(s) if present in the blood composition using the PIC (e.g., using the determined amount of the PIC) based at least in part on one or more of: a type of the blood composition, a volume of the blood composition, a type of PIC, a concentration of the PIC, a volume of the container for illumination, a number of platelets in the blood composition, a type of solution in the blood composition, and a weight of the blood composition. In some embodiments, the device is configured to calculate a light dose for subjecting the blood composition and PIC to ultraviolet light using the PIC based at least in part on a volume of the blood composition and a concentration of the PIC, and optionally a volume of the container for illumination. In some embodiments, the device is configured to transfer data indicating the calculated light dose to a second device or server. In some embodiments, the device is configured to transfer to a second device or server data to calculate a light dose for subjecting the blood composition and PIC to ultraviolet light to photochemically inactivate pathogen(s) if present in the blood composition using the PIC (e.g., the determined amount of the PIC). In some embodiments, the second device is an illumination device (e.g., configured to subject the blood composition and PIC to ultraviolet light). In some embodiments, the device further comprises one or more processors configured to determine an amount of the PIC to add to the blood composition to pathogen-inactivate the blood composition. In some embodiments, the one or more components configured to control flow of fluids comprise a pump and/or manifold. In some embodiments, the device further comprises one or more supports configured to support the PIC container and/or the blood composition container. In some embodiments, the device further comprises one or more fluid detectors configured to detect fluid transfer through the fluid path. In some embodiments, the device further comprises one or more fluid detectors configured to detect fluid transfer from the blood composition container into the mixing container. In some embodiments, the fluid path further comprises one or more of: a connector, a breakable connector, a cannula, a filter, a manifold, a pump, a container, and a coupling. In some embodiments, the fluid path further comprises one or more of: a connector, a breakable connector, a cannula, a filter, a manifold, a container, and a coupling. In some embodiments, the device is not an automated separation device (e.g., apheresis device). In some embodiments, the device is independent of an automated separation device (e.g., apheresis device). In some embodiments, the device is independent of an illumination device. In some embodiments, the processing set is configured to be reversibly coupled to one or more fluid paths, optionally, wherein the processing set comprises a fluid path (e.g., from a mixing container) configured for coupling (e.g., via connector(s)) to a first fluid path from a PIC container and/or a second fluid path from a quencher container; and/or wherein the processing set comprises a fluid path (e.g., from a mixing container) configured to be connected to a blood composition container; and/or wherein the processing set comprises one or more filters (e.g., 0.2 μm filters). In some embodiments, the fluid path is configured to be mounted on the device. In some embodiments, the system is configured to terminate fluid transfer from the PIC container into the mixing container via the first fluid path using one or more of the components configured to control flow of fluids based at least in part on detecting transfer of the determined amount of the PIC into the mixing container; optionally, based at least in part on detecting transfer of the determined amount of the PIC through the first fluid path using the one or more fluid detectors. In some embodiments, the system and/or fluid path comprises one or more of: a connector, a breakable connector, a cannula, a filter, a manifold, a pump, a container, and a coupling. In some embodiments, the device comprises a scale, and the system is configured to weigh the mixing container before, during, and/or after transfer of the determined amount of the PIC into the mixing container; optionally, wherein the system is configured to terminate fluid transfer from the PIC container into the mixing container via the using one or more of the components configured to control flow of fluids based at least in part on detecting transfer of a weight (e.g., detecting an increased weight) corresponding to the determined amount of the PIC into the mixing container using the scale. In some embodiments, the system is configured to transfer a determined amount of PIC to a processing set (e.g., mixing container) at a volume accuracy of within about 2% of the determined amount. In some embodiments, the system is configured to transfer and mix an amount of diluent solution and an amount of PIC, wherein the amount of PIC is transferred into the diluent, of the amount of diluent is transferred into the PIC, or the amount of PIC and the amount of diluent are transferred into a container, wherein the amount is transferred at a volume accuracy of within about 2% of a targeted volume. In some embodiments, the system is configured to transfer a determined amount of quencher to a processing set (e.g., mixing container) at a volume accuracy of within about 2% of the determined amount. In some embodiments, the system is configured to calculate a light dose for subjecting the blood composition and PIC to ultraviolet light to photochemically inactivate pathogen(s) if present in a blood composition using a determined amount of the PIC based at least in part on one or more of: a type of the blood composition, a volume of the blood composition, a concentration of the PIC, a volume of the container for illumination, a number of platelets in the blood composition, a type of solution in the blood composition, and a weight of the blood composition. In some embodiments, the system is configured to calculate a light dose for subjecting the blood composition and PIC to ultraviolet light to photochemically inactivate pathogen(s) if present in a blood composition using a determined amount of the PIC based at least in part on a volume of the blood composition and a concentration of the PIC, and optionally a volume of the container for illumination. In some embodiments, the system is configured to alert a system operator when a multi-dose (e.g. bulk) container of solution (e.g., PIC, quencher, processing solution, additive solution) should be replaced (e.g., based on elapsed time since coupling the container to the system, based on remaining volume in the container). In some embodiments, the system is configured to alert a system operator when a reusable consumable portion of the system should be replaced (e.g., based on elapsed time since loading the consumable portion, based on number of dosing cycles with the consumable portion).

It is to be understood that one, some, or all of the properties of the various embodiments described herein may be combined to form other embodiments. These and other aspects will become apparent to one of skill in the art. These and other embodiments are further described by the detailed description that follows.

### DESCRIPTION OF THE DRAWINGS

FIG. 1A provides a block diagram of an exemplary system, in accordance with some embodiments.

FIG. 1B illustrates an exemplary system, in accordance with some embodiments.

FIGS. 2A & 2B illustrate exemplary system use cases, in accordance with some embodiments.

FIG. 3 shows an exemplary processing set for red blood cells (RBCs), in accordance with some embodiments.

FIG. 4A shows a graph plotting the relationship between UVA light dose vs. volume for achieving low residual amotosalen levels remaining after photochemical treatment of a range of platelet volumes with amotosalen for pathogen inactivation.

FIG. 4B shows a graph plotting the relationship between UVA light dose vs. volume for achieving low residual amotosalen levels remaining after photochemical treatment of a range of platelet volumes with amotosalen for pathogen inactivation. Data are shown from experiments that used a 1.0 L or 1.3 L illumination container, as depicted.

FIG. 5A shows a graph plotting stability of S-303 over time after reconstitution in saline.

FIG. 5B shows a graph plotting stability of S-303 over time after reconstitution in saline.

FIG. 6 shows % hemolysis resulting from use of water (WFI) or saline to reconstitute S-303 and/or glutathione (GSH) post addition to RBCs. Left bars show use of WFI to reconstitute S-303 and GSH; middle bars show use of WFI to reconstitute GSH and saline to reconstitute S-303; and right bars show use of saline to reconstitute S-303 and GSH.

FIG. 7 shows % hemolysis resulting from use of water (WFI) to reconstitute S-303 at 28.8 mM or 50 mM, then dilute to 6 mM in saline prior to addition to RBCs. GSH was reconstituted in WFI in both test arms. For control, both S-303 and GSH were reconstituted in saline as standard practice.

FIG. 8 depicts an exemplary system for preparing a pathogen-inactivated blood composition, in accordance with some embodiments. The depicted system includes a device of the present disclosure (e.g., comprising one or more components configured to control flow of one or more fluids) and a processing set of the present disclosure.

FIG. 9 depicts an exemplary system for preparing a pathogen-inactivated blood composition, in accordance with some embodiments. The depicted system includes a device of the present disclosure (e.g., comprising one or more components configured to control flow of one or more fluids) and a processing set of the present disclosure.

FIG. 10 depicts an exemplary system for preparing a pathogen-inactivated blood composition, in accordance with some embodiments. The depicted system includes a device of the present disclosure (e.g., comprising one or more components configured to control flow of one or more fluids) and a processing set of the present disclosure.

FIG. 11 depicts an exemplary system for preparing a pathogen-inactivated blood composition, in accordance with some embodiments. The depicted system includes a device of the present disclosure (e.g., comprising one or more components configured to control flow of one or more fluids) and a processing set of the present disclosure.

FIG. 12 depicts an exemplary system for preparing a pathogen-inactivated blood composition, in accordance with some embodiments. The depicted system includes a device of the present disclosure (e.g., comprising one or more components configured to control flow of one or more fluids) and a processing set of the present disclosure.

FIG. 13 depicts an exemplary system for preparing a pathogen-inactivated blood composition, in accordance with some embodiments. The depicted system includes a device of the present disclosure (e.g., comprising one or more components configured to control flow of one or more fluids) and a processing set of the present disclosure.

FIG. 14 depicts an exemplary system for preparing a pathogen-inactivated blood composition, in accordance with some embodiments. The depicted system includes a device of the present disclosure (e.g., comprising one or more components configured to control flow of one or more fluids) and a processing set of the present disclosure.

FIG. 15 depicts an exemplary system for preparing a pathogen-inactivated blood composition, in accordance with some embodiments. The depicted system includes a device of the present disclosure (e.g., comprising one or more components configured to control flow of one or more fluids) and a processing set of the present disclosure.

FIG. 16 depicts an exemplary system for preparing a pathogen-inactivated blood composition, in accordance with some embodiments. The depicted system includes a device of the present disclosure (e.g., comprising one or more components configured to control flow of one or more fluids) and a processing set of the present disclosure.

FIG. 17 depicts an exemplary system for preparing a pathogen-inactivated blood composition, in accordance with some embodiments. The depicted system includes a device of the present disclosure (e.g., comprising one or more components configured to control flow of one or more fluids) and a processing set of the present disclosure.

FIG. 18 depicts an exemplary system for preparing a pathogen-inactivated blood composition, in accordance with some embodiments. The depicted system includes a device of the present disclosure (e.g., comprising one or more components configured to control flow of one or more fluids) and a processing set of the present disclosure.

FIG. 19 depicts an exemplary system for preparing a pathogen-inactivated blood composition, in accordance with some embodiments. The depicted system includes a device of the present disclosure (e.g., comprising one or more components configured to control flow of one or more fluids) and a processing set of the present disclosure.

FIG. 20 depicts an exemplary system for preparing a pathogen-inactivated blood composition, in accordance with some embodiments. The depicted system includes a device of the present disclosure (e.g., comprising one or more components configured to control flow of one or more fluids) and a processing set of the present disclosure.

FIG. 21 depicts an exemplary system for preparing a pathogen-inactivated blood composition, in accordance with some embodiments. The depicted system includes a device of the present disclosure (e.g., comprising one or more components configured to control flow of one or more fluids) and a processing set of the present disclosure.

FIG. 22 provides a flowchart that depicts an exemplary method for preparing a pathogen inactivated blood composition, in accordance with some embodiments.

### DETAILED DESCRIPTION

The present disclosure provides methods and systems for the preparation of pathogen-inactivated blood compositions and products. More particularly the present disclosure relates to methods of using devices (e.g., comprising component(s) configured to control flow of fluid(s)) to determine the amount of pathogen inactivation compound (PIC) necessary for pathogen inactivation of a blood composition (e.g., based at least in part on one or more input parameters) and allow transfer of the blood composition and the PIC into a container, e.g., of a processing set.

The use of the terms “a” and “an” and “the” and similar referents (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Wherever an open-ended term is used to describe a feature or element, it is specifically contemplated that a closed-ended term can be used in place of the open-ended term without departing from the spirit and scope of the disclosure. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the description and does not pose a limitation on the scope of the description unless otherwise claimed. No language in the specification should be construed as indicating any non-claimed element as essential to the practice of the methods, systems and compositions disclosed herein.

The term “pathogen inactivation” means a process useful to inactivate pathogens that may be present in a blood composition, where it is understood that the process does not necessarily inactivate completely all pathogens that may be present, but substantially reduces the amount of pathogens to significantly reduce the risk of a transfusion associated disease. The inactivation of a pathogen may be assayed by measuring the number of infective pathogen (e.g., viral or bacterial particles) in a certain volume, and the level of inactivation is typically represented in the log reduction in the infectivity of the pathogen, or log reduction in titer. Methods of assaying log reduction in titer, and measurements thereof for pathogen inactivation are known in the art. When the inactivation process is tested against a variety of pathogens, the reduction in a particular active pathogen is at least about 1 log, at least about 2 log, at least about 3 log, at least about 4 log, or at least about 5 log reduction in titer. A variety of pathogen inactivation processes are known in the art and may be used in the methods of the present disclosure, including for example, commercially available pathogen inactivation processes, such as INTERCEPT Blood System (Cerus Corp), Mirasol (TerumoBCT) and Theraflex (MacoPharma). In certain embodiments, pathogen inactivation may comprise treating with a pathogen inactivating compound. As used herein, “to pathogen-inactivate” is generally synonymous with “to inactivate a pathogen [i.e., in a blood composition of the present disclosure], if present.”

The term “pathogen inactivating compound (PIC)” means any suitable compound, such as a small organic compound, that can be used to inactivate a pathogen that may be present in a blood composition. Unless otherwise specified or clearly indicated by context, the PICs of the present disclosure are generally provided in a liquid form (e.g., in solution); however, in certain embodiments, a PIC can be provided in a solid form (e.g., dried, lyophilized, in a powder, etc.). For PICs in a solid form, reconstitution into a liquid form (e.g., in solution) is generally required before use.

A “photoactivated pathogen inactivation compound” is a suitable compound that requires some level of light in order to sufficiently inactivate a pathogen. Such compounds are preferred in some embodiments for the inactivation of pathogens in platelet products as they provide control over the inactivation process. Such photoactivated pathogen inactivation compounds described herein include psoralens, isoalloxazines, alloxazines, phthalocyanines, phenothiazines, and porphyrins, where these terms are understood to encompass a general class of compounds, i.e. the core compound and suitable derivatives thereof. For example psoralens or a psoralen generally describes the psoralen core compound and any derivative thereof (e.g. amotosalen), isoalloxazines or an isoalloxazine generally describes the isoalloxazine core and any derivative thereof (e.g. riboflavin), and so forth. Such derivatives comprise the core compound structure as well as additional substituents on the core. Descriptions of such compounds include any salts thereof.

The term “amotosalen” means the compound 3-(2-aminoethoxymethyl)-2,5,9-trimethylfuro[3,2-g]chromen-7-oneand any salts thereof. The compound may also be referred to as 4′-(4-amino-2-oxa)butyl-4,5′,8-trimethyl psoralen. The compound may also be referred to as 3-[(2-aminoethoxy) methyl]-2.5.9-trimethyl-7H-furo [3, 2-g][1]benzopyran-7-one. Where the inactivation of platelets includes adding amotosalen HCl (the HCl salt of amotosalen) to a unit of platelets, the removal of this compound from the unit of platelets is not limited to the removal of amotosalen HCl, as the amotosalen can be present in solution as other salts or as the free base. As used in the methods described herein, removal of amotosalen means removal of the compound in any form, e.g. as the free base or as any salt, as measured by the assays described herein.

Other pathogen-inactivating compounds that may be used by the methods of the disclosure include compounds that comprise a functional group which is, or which is capable of forming and has formed, e.g. in situ, a reactive group, such as an electrophilic group. In some cases, the pathogen-inactivating compounds of the present disclosure do not require photoactivation to be reactive. For example, the functional group may be a mustard group, a mustard group intermediate, a mustard group equivalent, an epoxide, a formaldehyde or a formaldehyde synthon. Such functional groups are capable of forming in situ a reactive group, such as an electrophilic aziridine, aziridinium, thiirane or thiiranium ion. A mustard group may be a mono- or bis-(haloethyl)amine group or a mono (haloethyl)sulfide group. A mustard equivalent is a group that reacts by a mechanism similar to the mustards, for example by forming reactive intermediates such as aziridinium and aziridine groups or thiirane and thiiranium groups. Examples include aziridine derivatives, mono or bis-(mesylethyl)amine groups, mono-(mesylethyl)sulfide groups, mono or bis-(tosylethyl)amine groups and mono-(tosylethyl)sulfide groups. A formaldehyde synthon is any compound that breaks down to a formaldehyde, which includes a hydroxylamine such as hydroxymethylglycine. The reactive group of the pathogen-inactivating compound is capable of reacting with the nucleic acids of pathogens, for example with nucleophilic groups on the nucleic acid. The reactive group is also capable of reacting with a nucleophilic group of a quencher. Pathogen-inactivating compounds may also include a component that targets the compound to nucleic acids, such as an anchor portion. The anchor portion comprises a moiety which is capable of binding non-covalently to a nucleic acid biopolymer, such as DNA or RNA, and is also referred to as a nucleic acid binding ligand, nucleic acid binding group, or nucleic acid binding moiety. Examples of such compounds are described in U.S. Pat. Nos. 5,691,132, 6,410,219, 6,136,586, 6,617,157, and 6,709,810, each of which is incorporated by reference herein. Another class of pathogen-inactivating compounds that may be quenched by the methods of the disclosure comprises the above-mentioned reactive groups linked to a nucleic acid binding group via a hydrolysable linker, as described in U.S. Pat. No. 6,514,987, incorporated by reference herein. The anchor portion of the pathogen-inactivating compounds has an affinity for nucleic acids. This affinity may be due to any of several modes of binding to the nucleic acid non-covalently, including, but not limited to, intercalation, minor groove binding, major groove binding, and electrostatic binding (e.g., phosphate backbone binding). The affinity may also be due to mixed modes of binding (e.g., intercalation and minor groove binding). The binding may be sequence-specific (i.e., increased binding affinity for one or more particular nucleic acid sequences over other nucleic acid sequences) or non sequence-specific. Detailed examples of such nucleic acid binding moieties can be found in the above-mentioned patents.

### Methods

Certain aspects of the present disclosure provide methods of preparing a pathogen-inactivated blood composition. In some embodiments, the methods comprise, e.g., at a device comprising one or more components configured to control flow of one or more fluids: coupling a container containing a pathogen inactivation compound (PIC container) to a first fluid path; coupling (e.g., connecting) a processing set to the first fluid path; transferring a blood composition from a container containing the blood composition (blood composition container) into a container of the processing set; determining an amount of a pathogen inactivation compound (PIC) to pathogen-inactivate the blood composition based at least in part on one or more input parameters; and transferring the determined amount of the PIC from the PIC container into the container of the processing set via the first fluid path using the one or more components configured to control flow of fluids. In some embodiments, the methods comprise, e.g., at a device comprising one or more components configured to control flow of one or more fluids: coupling a container containing a pathogen inactivation compound (PIC container) to a first fluid path; coupling (e.g., connecting) a processing set to the first fluid path; transferring a blood composition from a container containing the blood composition (blood composition container) into a container of the processing set; determining an amount of a pathogen inactivation compound (PIC) to inactivate a pathogen in the blood composition, if present, based at least in part on one or more input parameters; and transferring the determined amount of the PIC from the PIC container into the container of the processing set via the first fluid path using the one or more components configured to control flow of fluids.

In some embodiments, the methods comprise, e.g., at a device comprising one or more components configured to control flow of one or more fluids: coupling (e.g., connecting) a container containing a quencher to a first fluid path; coupling (e.g., connecting) a processing set to the first fluid path; transferring a blood composition from a container containing the blood composition (blood composition container) into a container of the processing set; determining an amount of quencher to quench a PIC based at least in part on one or more input parameters; and transferring the determined amount of the quencher from the quencher container into the container of the processing set via the first fluid path using the one or more components configured to control flow of fluids.

In some embodiments, the amount of the PIC (and/or quencher) to pathogen-inactivate the blood composition is determined based at least in part on weight and/or volume of the blood composition. In some embodiments, the amount of the PIC (and/or quencher) to pathogen-inactivate the blood composition is determined based at least in part by calculating the volume of the blood composition based on the weight of the blood composition (e.g., the weight of the blood composition in the blood composition container).

In some embodiments, determining the amount of the PIC (and/or quencher) to pathogen-inactivate the blood composition further comprises weighing the blood composition, e.g., in the blood composition container. In some embodiments, the weight of the blood composition container is subtracted before or after weighing the blood composition in the blood composition container. In some embodiments, a tare weight of the blood composition container (e.g., without a blood composition present) is obtained prior to weighing the blood composition in the blood composition container or is based on a known weight of the blood composition container. In some embodiments, the blood composition is weighed in the blood composition container using a scale, e.g., of a device of the present disclosure.

In some embodiments, determining the amount of the PIC (and/or quencher) to pathogen-inactivate the blood composition further comprises receiving an input indicating the weight of the blood composition, e.g., from a scale of a device of the present disclosure. In some embodiments, the input further indicates the determined amount of the PIC (and/or quencher). In some embodiments, the received input can include identifying information associated with the blood composition from a barcode scanner (e.g., a barcode), QR code scanner (e.g., a QR code), radio frequency identification (RFID) scanner (e.g., an RFID), data representing the weight of the blood composition (e.g., from a scale), or a user input (e.g., from a touchscreen display, keyboard, or mouse).

In some embodiments, one or more input parameters (e.g., used as part of determining the amount of PIC (and/or quencher) to pathogen-inactivate a blood composition of the present disclosure) comprise one or more of: a type of the blood composition (i.e., the component(s) of the blood composition, such as platelets, plasma, or RBCs; not ABO blood type), a volume of the blood composition, a number of platelets or red blood cells in the blood composition, a type of solution in the blood composition (e.g., plasma and/or platelet additive solution (PAS) or other additive solution, etc.), and a weight of the blood composition.

In some embodiments, a device of the present disclosure determines the amount of the PIC (and/or quencher) to pathogen-inactivate the blood composition, e.g., using one or more processors. In other embodiments, the amount of the PIC (and/or quencher) to pathogen-inactivate the blood composition is input into the device, e.g., via received input of the present disclosure.

In some embodiments, the methods of the present disclosure further comprise reconstituting the PIC (and/or quencher) in a solution. For example, for certain PICs (and/or quenchers) of the present disclosure such as those used with a quencher, the PIC (and/or quencher) can be contained in a dual (e.g., two, double) chamber container (e.g., bag, syringe) that comprises a first chamber containing the dry form of PIC (or quencher) and a second chamber comprising the solution/diluent (e.g., separated by an openable or breakable barrier or seal). Examples of solution/diluent may include saline (e.g., 0.9% NaCl), water (e.g., water for injection, WFI), buffers, additives, etc.

In some embodiments, a blood composition of the present disclosure is transferred into the container of the processing set via a second fluid path. In some embodiments, a blood composition of the present disclosure is transferred into the container using gravity flow. In other embodiments, a blood composition of the present disclosure is transferred into the container, e.g., by the device, such as using a pump. In some embodiments, the determined amount of the PIC is transferred into the container via the first fluid path using a pump. In some embodiments, a determined amount of the quencher is transferred into the container via a fluid path using a pump. In some embodiments, the blood composition is transferred into the container (e.g., via a second fluid path) using a pump.

In some embodiments, the methods of the present disclosure further comprise (e.g., after transferring the determined amount of the PIC from the PIC container (and/or a determined amount of quencher from the quencher container) into the container of the processing set) terminating fluid transfer from the PIC container (and/or quencher container) into the container of the processing set (e.g., via the first fluid path) using one or more of the components configured to control flow of fluids (e.g., of the device). In some embodiments, the termination is based at least in part on a signal from a pump or a time period after activation of the pump. In some embodiments, the termination is based at least in part on the pump transferring PIC and/or quencher for the required amount of time as determined by flow rate. In some embodiments, the termination is based at least in part on detecting transfer of the determined amount of the PIC (and/or quencher) into the container of the processing set, e.g., using one or more fluid detectors. In some embodiments, the termination is based at least in part on detecting transfer of a weight corresponding to the determined amount of the PIC (and/or quencher) into the container of the processing set, e.g., using a scale.

In some embodiments, the methods of the present disclosure further comprise detecting transfer of the determined amount of the PIC (and/or quencher) into the container of the processing set (e.g., via the first fluid path), such as by using one or more fluid detectors in operable linkage with the first fluid path or by using one or more fluid detectors external to or noncontact with the first fluid path. In some embodiments, transfer of the determined amount of the PIC into the container of the processing set via the first fluid path is detected by measuring a volume of fluid transferred through the first fluid path using the one or more fluid detectors.

In some embodiments, the methods of the present disclosure further comprise detecting transfer of the blood composition into the container of the processing set (e.g., via a second fluid path), such as by using one or more fluid detectors in operable linkage with the fluid path or by using one or more fluid detectors external to or noncontact with the first fluid path. In some embodiments, transfer of the blood composition into the container of the processing set via the fluid path is detected by measuring a volume of fluid transferred through the fluid path using the one or more fluid detectors.

In some embodiments, the methods of the present disclosure further comprise (e.g., after transferring the blood composition from the blood composition container into the container) terminating fluid transfer from the blood composition container into the container of the processing set (e.g., via a second fluid path) using one or more of the components configured to control flow of fluids (e.g., of the device). In some embodiments, the termination is based at least in part on detecting transfer of the blood composition into the mixing container.

In some embodiments, the methods of the present disclosure further comprise weighing the container of the processing set during and/or after transfer of the determined amount of the PIC into the container, e.g., using a scale of a device of the present disclosure.

In some embodiments, the methods of the present disclosure further comprise coupling a container containing a diluent (diluent container) to a fluid path of the present disclosure, e.g., the first fluid path. In some embodiments, a quencher of the present disclosure can be contained in a dual (e.g., two, double) chamber container (e.g., bag, syringe) that comprises a first chamber containing the dry form of PIC (and/or a quencher) and a second chamber comprising a diluent (e.g., separated by an openable or breakable barrier or seal). In some embodiments, a container of diluent and a container of the dry form of PIC (and/or quencher) are connected to the device, and the device transfers diluent to the PIC (and/or quencher) and resuspends, prior to dosing. Examples of solution/diluent may include saline, water (e.g., water for injection, WFI), buffers, additives, etc.

Transferring the determined amount of the PIC from the PIC container into the container of the processing set via the first fluid path can be accomplished directly between the PIC container into the container of the processing set or through one or more other containers. In some embodiments, transferring the determined amount of the PIC from the PIC container into the container via the first fluid path comprises: transferring diluent from a diluent container into the PIC container; and transferring an amount of the diluent solution and the PIC (e.g., diluted PIC) to provide the determined amount of the PIC from the PIC container into the container of the processing set. In some embodiments, transferring the determined amount of the PIC from the PIC container into the container via the first fluid path comprises: transferring PIC from the PIC container into the diluent container; and transferring an amount of the diluent solution and the PIC (e.g., diluted PIC) to provide the determined amount of the PIC from the diluent container into the container of the processing set. In some embodiments, transferring the determined amount of the PIC from the PIC container into the container of the processing set via the first fluid path comprises: transferring diluent from the diluent container into a third container, wherein the first fluid path further comprises the third container; transferring PIC from the PIC container into the third container; and transferring diluent and the determined amount of the PIC from the third container into the container of the processing set. In some embodiments, transferring the determined amount of the PIC from the PIC container into the mixing container via the first fluid path comprises: transferring diluent from the diluent container into a third container; transferring PIC from the PIC container into the third container; and transferring diluent and the determined amount of the PIC (e.g., diluted PIC) from the third container into the container of the processing set. In some embodiments, the third container is coupled or configured to be coupled to the first fluid path. In some embodiments, the first fluid path comprises the third container. In some embodiments, transferring the determined amount of the PIC from the PIC container into the mixing container via the first fluid path comprises: transferring diluent from the diluent container into a third container, wherein the first fluid path comprises the third container; transferring PIC from the PIC container into the third container; and transferring diluent and the determined amount of the PIC (e.g., diluted PIC) from the third container into the container of the processing set. In some embodiments, transferring the determined amount of the PIC from the PIC container into the container of the processing set via the first fluid path comprises: coupling a third container to the first fluid path; transferring diluent from the diluent container into the third container; transferring PIC from the PIC container into the third container; and transferring diluent and the determined amount of the PIC (e.g., diluted PIC) from the third container into the container of the processing set. In some embodiments, the PIC in the PIC container is present at a concentration at least 2 times, at least about 3 times, at least about 4 times, at least about 5 times, at least about 6 times, at least about 7 times, at least about 8 times, at least about 9 times, or at least about 10 times or more greater than required to provide a solution comprising the diluent and the determined amount of the PIC (e.g., diluted PIC). In some embodiments, the PIC is diluted with the diluent at least about 2-fold, at least about 3-fold, at least about 4-fold, at least about 5-fold, at least about 6-fold, at least about 7-fold, at least about 8-fold, at least about 9-fold, at least about 10-fold or more.

A goal of the devices and methods of the present disclosure is to provide accurate transfer of liquids, such as transfer of the determined amount of the PIC, quencher, and/or diluent solution into the container of the processing set, the transfer of PIC into diluent, diluent into PIC or PIC and diluent into another container, or transfer of the blood composition into the container of the processing set. In some embodiments, the determined amount of the PIC is transferred into the container at a volume accuracy of within about 1%, within about 2%, within about 3%, within about 4%, or within about 5% of the determined amount. For example, using a 50 mM starting solution of PIC (amustaline), 2.22±0.04 mL of the PIC solution can be transferred into the container. Alternatively, using a 28.8 mM starting solution of PIC (amustaline), 3.85±0.08 mL of the PIC solution can be transferred into the container. In some embodiments, 18.5 mL PIC (amustaline) can be transferred into the container with an accuracy of ±0.3 mL. As another example, 10 mL to 25 mL of 52.3 μM to 57.8 μM amotosalen can be transferred into the container at a volume accuracy of within about 1%, within about 2%, within about 3%, within about 4%, or within about 5%. In some embodiments, the determined amount of the PIC (e.g., amotosalen) is transferred into the container with an accuracy in volume of <5% based on a target dose established from the volume or mass of input platelets within about 1%, within about 2%, within about 3%, within about 4%, or within about 5% of the determined amount. In some embodiments, amount of a diluent solution is transferred into the container at a volume accuracy of within about 1%, within about 2%, within about 3%, within about 4%, or within about 5% of a targeted volume. In some embodiments, quencher or a solution thereof is transferred into the container at a volume accuracy of within about 1%, within about 2%, within about 3%, within about 4%, or within about 5% of the determined amount. For example, 15 mL of quencher (e.g., glutathione) can be transferred into the container with an accuracy of ±0.3 mL. In some embodiments, PIC is transferred into diluent, diluent is transferred into PIC, or PIC and diluent are transferred into another (e.g., third) container at a volume accuracy of within about 1%, within about 2%, within about 3%, within about 4%, or within about 5% of a targeted volume.

In some embodiments, after the determined amount of the PIC is transferred into the container of the processing set via the first fluid path, less than about 0.1 mL, less than about 0.2 mL, less than about 0.3 mL, less than about 0.4 mL, or less than about 0.5 mL of residual fluid is remaining in the first fluid path.

In some embodiments, the methods of the present disclosure further comprise (e.g., after or concurrently with transferring a determined amount of quencher from a quencher container into the mixing container) admixing the blood composition and the determined amount of the quencher in the container of the processing set. In some embodiments, the methods of the present disclosure further comprise (e.g., after or concurrently with transferring the determined amount of the PIC from the PIC container into the container of the processing set) admixing the blood composition and the determined amount of the PIC in the container of the processing set. For example, the container of the processing set can be rocked or agitated. In some embodiments, a device of the present disclosure is configured to agitate or rock the container of the processing set, e.g., after or concurrently with transferring the determined amount of the PIC (and/or quencher) into the container of the processing set. For example, the container of the processing set can be agitated at a speed between 10 and 40 RPM, between 10 and 30 RPM, between 14 and 22 RPM and/or rocked at an angle of rocking 0° and 90°, between 10° and 80°, between 30° and 70°, between 35° and 65°, between 38° and 58°. In some embodiments, the container of the processing set can be rocked or agitated on a scale of the device.

In some embodiments, transferring the determined amount of the PIC from the PIC container (and/or quencher from the quencher container) into the container of the processing set can comprise use of gravity flow. In some embodiments, transferring the determined amount of the PIC from the PIC container (and/or quencher from the quencher container) into the container of the processing set (e.g., via the first fluid path) can comprise use of one or more pumps.

In some embodiments, the methods of the present disclosure further comprise (e.g., after transferring the determined amount of the PIC from the PIC container into the container of the processing set) subjecting the blood composition in admixture with the PIC to illumination (e.g., photochemical inactivation of one or more pathogens, if present) using the determined amount of the PIC, e.g., in the container of the processing set. In some embodiments, the methods of the present disclosure further comprise (e.g., after subjecting the blood composition in admixture with the PIC to illumination) transferring the pathogen-inactivated blood composition into one or more storage containers. In some embodiments, subjecting the blood composition to illumination comprises illuminating for a duration of between 1 second and 2 hours; and/or illuminating at an intensity between 1 and 1000 mW/cm²; and/or illuminating with a total dose of ultraviolet light between about 0.5 J/cm² to about 50 J/cm².

In some embodiments, illumination is with light having a peak wavelength in an ultraviolet A spectrum (e.g., 315-400 nm). In some embodiments, the peak wavelength is from about 315 nm to about 350 nm. In some embodiments, the peak wavelength is from about 315 nm to about 335 nm. In some embodiments, the peak wavelength is from about 320 nm to about 330 nm. In some embodiments, the peak wavelength is from about 330 nm to about 350 nm. In some embodiments, the peak wavelength is from about 340 nm to about 350 nm. In some embodiments, the peak wavelength is in an ultraviolet A spectrum (e.g., 315-400 nm), and ultraviolet B spectrum (e.g., 280-315 nm) or an ultraviolet C spectrum (e.g., 100-280 nm, 200-280 nm, 240-280 nm). In some embodiments, the light for illumination is from light sources that are light emitting diodes (LEDs). In some embodiments, the light for illumination is from light sources, wherein the light intensity at 50% of the maximum peak intensity of light emitted by the light sources is within a spectral width of less than 20 nanometers of the peak wavelength (e.g., no more than 10 nanometers greater than, no more than 10 nanometers less than the peak wavelength; within 10 nanometers less than, within 10 nanometers greater than the peak wavelength). In some embodiments, the full-width half-maximum (FWHM) spectral width (e.g., bandwidth) of light (e.g., spectral bandwidth at the maximum peak intensity) from light sources for illumination is within 20 nanometers of the peak wavelength (e.g., no more than 10 nanometers greater than, no more than 10 nanometers less than the peak wavelength; within 10 nanometers less than, within 10 nanometers greater than the peak wavelength).

In some embodiments, the methods do not comprise contacting the pathogen-inactivated blood composition with a compound adsorption device (CAD). In other embodiments, the methods of the present disclosure further comprise (e.g., after subjecting the blood composition in admixture with the PIC to illumination) transferring the pathogen-inactivated blood composition from the container into a container containing a CAD.

In some embodiments, the methods of the present disclosure further comprise calculating a light dose for photochemical inactivation of the blood composition using the determined amount of the PIC based at least in part on one or more of: a type of the blood composition (i.e., the component(s) of the blood composition, such as platelets, plasma, or RBCs; not ABO blood type), a volume of the blood composition, a number of platelets in the blood composition, a type of solution in the blood composition (e.g., plasma and/or platelet additive solution (PAS) or other additive solution, etc.), and a weight of the blood composition. In some embodiments, the methods of the present disclosure further comprise transferring data indicating the calculated light dose to a second device (e.g., an illumination device) or server, e.g., via wired or wireless connection.

### Biological Fluids, Blood Compositions, and Pathogen Inactivation

The methods, devices, and systems disclosed herein are contemplated for use in pathogen inactivation of a variety of biological fluids, such as for example, blood compositions, products, and components, including platelets, plasma, RBCs, and whole blood.

As used herein, a “biological fluid” refers to any fluid that is found in or derived from an organism (e.g., human, animal, plant, microorganism), or that comprises one or more components (e.g., biologics) found in, isolated from, or derived from an organism, including synthetic versions thereof. Biological fluids may include, but are not limited to blood compositions, products and components (e.g., platelets, plasma, red blood cells (RBC), whole blood, cryoprecipitate, cryo-reduced plasma), vaccines, cells (e.g., primary cells, cell lines, cell cultures), natural and recombinant proteins (e.g., therapeutics, antibodies), bacterial cultures, virus suspensions and the like. In some embodiments, a biological fluid (e.g., blood composition) may further comprise a non-biological fluid, such as for example, a physiological solution (e.g., diluent solution), including but not limited to saline, buffered solution, nutrient solution, platelet additive solution (PAS), and/or anticoagulant solution.

Additive solutions, such as platelet additive solutions and red blood cell additive solutions are contemplated for use in the systems and methods of the present disclosure. Many such additive solutions are well known in the art, including the following non-limiting examples of platelet additive solutions: PAS with or without citrate, PAS-A, PAS-B, PAS-C, PAS-D, PAS-E, PAS-F, Composol, SSP, SSP+, Intersol, Isoplate, T-Sol, PlasmaLyte, PAS II, PAS III, PAS HIM, and as non-limiting examples of red blood cell additive solutions: AS-1, AS-3, AS-5, AS-7, SAGM, PAGGSM, Adsol, Nutricel, Optisol, MAP, E-sol. In some embodiments, the additive solution is approved (e.g., for human use products) by a regulatory agency, such as for example, a regulatory agency in a country or region where the systems and methods disclosed in the present application may be used

In some embodiments, a blood composition can be a whole blood composition or a composition comprising one or more blood components, e.g., platelets, RBCs, plasma, cryoprecipitate, cryo-reduced plasma, and so forth. In some embodiments, a blood composition of the present disclosure comprises platelets and/or plasma, e.g., a platelet and/or plasma composition. In some embodiments, a blood composition of the present disclosure comprises red blood cells (RBCs). Suitable PICs, methods, and processing sets for pathogen inactivation of platelets and/or plasma, red blood cells, whole blood, cryoprecipitate and/or cryo-reduced plasma are described herein.

In some embodiments, after transferring the determined amount of the PIC from the PIC container into the mixing container, the PIC is at a concentration of 25 μM to about 1500 μM, about 50 μM to about 1000 μM, about 50 μM to about 500 μM, about 50 μM to about 250 μM, about 50 μM to about 150 μM. In some embodiments, the concentration of PIC is at a concentration about 40 μM to about 80 μM, 40 μM to about 70 μM, 40 μM to about 60 μM, 50 μM to about 70 μM, or 50 μM to about 60 μM. In some embodiments, the concentration of PIC is at a concentration about 40 μM, about 45 μM, about 50 μM, about 55 μM, about 60 μM, about 65 μM, about 70 μM, about 75 μM, or about 80 μM in the mixing container with the blood composition.

In some embodiments, a blood composition of the present disclosure comprises RBCs, e.g., a RBC composition. Suitable PICs, quenchers, methods, and processing sets for pathogen inactivation of RBCs are described herein.

In some embodiments, after transferring the determined amount of the PIC from the PIC container into the mixing container, the PIC is at a concentration of about 0.05 mM to 4 mM, about 0.05 mM to 2 mM, about 0.05 mM to 0.5 mM, about 0.1 mM to 0.3 mM, or about 0.05 mM, 0.1 mM, about 0.2 mM, about 0.3 mM, about 0.4 mM or about 0.5 mM in the mixing container with the blood composition. In some embodiments, after transferring the an amount of quencher from the quencher container into the mixing container, the quencher is at a concentration of about 0.05 mM to 30 mM, about 0.1 mM to 25 mM, about 1 mM to 20 mM, about 2 mM to 20 mM, or about 2 mM, about 4 mM, about 6 mM, about 8 mM, about 10 mM, about 12 mM, about 14 mM, about 16 mM, about 18 mM or about 20 mM in the mixing container with the blood composition.

In some embodiments, the methods of the present disclosure further comprise mixing a blood composition (e.g., comprising RBCs) with a quencher. In some embodiments, mixing the blood composition with a quencher comprises: at a second device comprising one or more components configured to control flow of one or more fluids: (i) coupling a container containing a quencher (quencher container) to a first fluid path; (ii) coupling a mixing container to the first fluid path; (iii) determining an amount of quencher to quench a pathogen inactivation compound (PIC) based at least in part on one or more input parameters; (iv) transferring the determined amount of the quencher from the quencher container into the mixing container using one or more of the components configured to control flow of fluids of the second device; and (v) transferring the blood composition from a container containing the blood composition (blood composition container) into the mixing container. In some embodiments, the quencher is added to the mixing container before the blood composition is added to the mixing container. In some embodiments, the quencher and blood composition are mixed prior to mixing the PIC and blood composition. In some embodiments, the methods further comprise uncoupling the mixing container containing the blood composition and the quencher from the second device and coupling the uncoupled mixing container to the first device. In some embodiments, the methods further comprise using the second device to determine an amount of the quencher to pathogen-inactivate the blood composition (e.g., in combination with a PIC of the present disclosure) based at least in part on one or more input parameters, e.g., as set forth herein. For example, the second device can be configured similar to any of the devices of the present disclosure, but with a quencher container instead of a PIC container. In some embodiments, the blood composition container is coupled to the second device prior to the methods of the present disclosure (i.e., the method using the quencher container device is carried out prior to the methods of preparing a pathogen inactivated blood composition as described herein).

In some embodiments, mixing the blood composition with a quencher comprises coupling a quencher container to a fluid path (e.g., a second or third fluid path); and transferring quencher from the quencher container into the mixing container via the fluid path (e.g., the second or third fluid path) using one or more of the components configured to control flow of fluids. In some embodiments, the quencher is added to the mixing container before the blood composition is added to the mixing container. In some embodiments, the quencher and blood composition are mixed prior to mixing the PIC and blood composition.

In some embodiments, the methods of the present disclosure further comprise reconstituting a quencher in a solution, e.g., prior to transferring the quencher from the quencher container into the blood composition container and/or mixing container.

### Pathogen Inactivation of Platelets/Plasma

Blood compositions, products, and components may contain pathogens, or may be contaminated with pathogens during processing. As such, it is desirable to subject such blood products to a pathogen inactivation process in order to reduce the risk of transfusion-transmitted diseases. Various processes and methods have been assessed to mitigate the risk of transfusion-associated disease transmission in blood products. Aside from screening and detection of pathogens and subsequent elimination of contaminated blood products, processes that incorporate treatments to inactivate pathogens (i.e., pathogen inactivation) that may be present are available. Ideally, such a process results in the inactivation of a broad range of pathogens such as viruses, bacteria and parasites that may be present in the blood product. A pathogen inactivation compound may also be referred to as a pathogen inactivating compound.

In certain embodiments, the methods of pathogen inactivation require addition of an amount of pathogen inactivating compound to a blood composition, such as those comprising plasma and/or platelets (e.g., treating a platelet preparation). For example, pathogen inactivation may involve the addition of a low molecular weight compound that inactivates various pathogens, where a preferred method involves the addition of a photosensitizer that, when activated by illumination using light of suitable wavelengths, will inactivate a variety of pathogens that may be present. Two preferred methods that are commercially available include the addition of amotosalen or riboflavin to the platelets, with subsequent illumination with UV light. Other methods include illumination with UV light without addition of a photosensitizer, as well as illumination with other photoactive compounds, including psoralen derivatives other than amotosalen, isoalloxazines other than riboflavin, alloxazines, dyes such as phthalocyanines, phenothiazine dyes (e.g. methylene blue, azure B, azure C, thionine, toluidine blue), porphyrin derivatives (e.g. dihematoporphyrin ether, hematoporphyrin derivatives, benzoporphyrin derivatives, alkyl-substituted sapphyrin), and merocyanine 540 (Prodouz et al., Blood Cells 1992, 18(1):101-14; Sofer, Gail, BioPharm, August 2002). Other pathogen inactivation systems include, for example, those described in PCT publication numbers WO 2012071135; WO 2012018484; WO 2003090794; WO 2003049784; WO 1998018908; WO 1998030327; WO 1996008965; WO 1996039815; WO 1996039820; WO 1996040857; WO 1993000005; US patent application number US 20050202395; and U.S. Pat. Nos. 8,296,071 and 6,548,242, the disclosures of which are hereby incorporated by reference as they relate to pathogen inactivation in blood products. In some embodiments, the pathogen inactivating compound is a photoactive pathogen inactivating compound selected from the group consisting of a psoralen, an isoalloxazine, an alloxazine, a phthalocyanine, a phenothiazine, a porphyrin, and merocyanine 540. In some embodiments, the pathogen inactivating compound is a psoralen. In some embodiments, the pathogen inactivating compound is amotosalen. Where addition of a compound to the platelets is used for pathogen inactivation, whether the method requires illumination or not, in some instances it is desirable to remove any residual pathogen inactivation compound or by-product thereof.

Methods for pathogen inactivation and removal of pathogen inactivating compound as described herein are applicable to any platelet preparations, whether the platelet preparations comprise individual platelet donations (e.g., apheresis collected platelets) or pooled platelet preparations. These processes typically provide a platelet preparation that is either in about 85% to 100% plasma or has some amount of platelet additive solution added, typically in the range of 50 to 95% platelet additive solution, with the rest of the volume effectively being plasma, i.e. plasma in the range of about 5 to 50%. It is understood that a solution of pathogen inactivating compound can be added during the processing to inactivate pathogens, since pathogen inactivating compound is not typically combined in solid form, but is dissolved in a solution (for example, amotosalen is the HCl salt dissolved in a saline solution). As such, in some instances, when a platelet preparation is designated as about 100% plasma, it is understood that this means no additional platelet additive solution is included in the platelet unit. If such a preparation of platelets in about 100% plasma is treated for pathogen inactivation, some volume of the solution of pathogen inactivating compound will be included in the final product, as well as some volume of anticoagulant used in collecting the blood for isolation of platelets. While the plasma has been diluted partially with whatever amount of anticoagulant and solution that is used to contain the pathogen inactivating compound, the resulting platelet preparation including pathogen inactivation compound may be referred to as comprising about 100% plasma, or may be referred to as about 85 to 100% plasma (typically less than about 5 to 15% of the volume will comprise the solution used to deliver the pathogen inactivating compound). Platelet preparations can also be prepared with some amount of platelet additive solution, which may, for example, be added after concentrating the platelets, removing a portion of the plasma from the supernatant, and adding the desired amount of platelet additive solution to the platelet preparation. The platelet additive is added to provide the desired percentage of platelet additive solution. Such a preparation of platelets is typically adjusted so the plasma content is about 5 to 50%, with the remainder of the solution being platelet additive solution, i.e. 50 to 95% platelet additive solution. When amounts of plasma and platelet additive solutions are described, it is understood that as with platelet preparations described as being in about 100% plasma, some volume of solution containing a pathogen inactivating compound may be included in the unit of platelets containing a pathogen inactivating compound. While the solution has been diluted partially with whatever amount of solution is used to contain the pathogen inactivating compound, it is understood that, for example, a platelet preparation designated as comprising 35% plasma and 65% platelet additive solution may refer to relative amounts of plasma and platelet additive solution prior to the addition of a solution containing pathogen inactivating compound.

Some pathogen inactivation methods may require the use of a removal device, i.e. a device for reducing the concentration of pathogen inactivating compound, such as a small organic compound, e.g. platelet inactivating compound, and by-products thereof in a preparation of platelets, while substantially maintaining a desired biological activity of the platelets. In some embodiments, the removal device is referred to as a compound adsorption device (CAD), and may comprise a container (e.g., CAD container, CAD bag) containing one or more materials, such as for example, adsorbent particles, and which is suitable for also containing a preparation of platelets from which the concentration of pathogen inactivating compound and by-products thereof are to be reduced. Such a removal device is generally intended to be used in a batch mode, i.e. the device is placed in contact with the platelets, and continued contact with the removal device, e.g. with shaking to allow essentially the entirety of the solution of platelets to come into contact with the removal device over time of contact, results in reducing the levels of pathogen inactivating compound. Such batch devices entail the use of an adsorbent particle that binds the pathogen inactivation compound, and can be used by either adding adsorbent particles directly to the platelet container (e.g., bag) following illumination or transferring the platelets to a bag containing the adsorbent particles following illumination and the platelets are then agitated for a specified period of time with the platelet preparations contacting the removal device. While free adsorbent particles may be used as a removal device, such particles may be contained within a mesh pouch, such as a polyester or nylon mesh pouch, which allows for contact of the platelet solution with the adsorbent particles while containing the particles within the pouch. Alternatively, the adsorbent particles may be immobilized within a matrix, where the immobilized matrix can reside directly in the blood bag used for batch removal, or may be similarly contained within a mesh pouch. In some instances, the removal device comprises porous adsorbent particles in an amount sufficient to reduce the pathogen inactivating compound to below a desired concentration, wherein the adsorbent particles have an affinity for the pathogen inactivating compound, where it is understood such adsorbent particle can be selected to best adsorb the compound or compounds to be removed, with minimal effect on components that should not be removed or damaged by contact with the adsorbent particle. A variety of adsorbent particles are known, including generally particles made from any natural or synthetic material capable of interacting with compounds to be removed, including particulates made of natural materials such as activated carbon, silica, diatomaceous earth, and cellulose, and synthetic materials such as hydrophobic resins, hydrophilic resins or ion exchange resins. Such synthetic resins include, for example, carbonaceous materials, polystyrene, polyacrylic, polyacrylic ester, cation exchange resin, and polystyrene-divinylbenzene. Detailed description of such removal devices suitable for use in the methods as described herein can be found in PCT publication numbers WO 1996040857, WO 1998030327, WO 1999034914, and WO 2003078023, the disclosures of which are hereby incorporated by reference with respect to the discussion of such removal devices and the adsorbent particles and other materials used to prepare such devices. Exemplary adsorbent particles include, but are not limited to, Amberlite (Rohm and Haas) XAD-2, XAD-4, XAD-7, XAD-16, XAD-18, XAD-1180, XAD-1600, XAD-2000, XAD-2010; Amberchrom (Toso Haas) CG-71m, CG-71c, CG-161m, CG161c; Diaion Sepabeads (Mitsubishi Chemicals) HP20, SP206, SP207, SP850, HP2MG, HP20SS, SP20MS; Dowex (Dow Chemical) XUS-40285, XUS-40323, XUS-43493 (also referred to as Optipore V493 (dry form) or Optipore L493 (hydrated form)), Optipore V503, Optipore SD-2; Hypersol Macronet (Purolite) MN-100, MN-102, MN-150, MN-152, MN-170, MN-200, MN-202, MN-250, MN-252, MN-270, MN-300, MN-400, MN-500, MN-502, Purosorb (Purolite) PAD 350, PAD 400, PAD 428, PAD 500, PAD 550, PAD 600, PAD 700, PAD 900, and PAD 950. The material used to form the immobilized matrix comprises a low melting polymer, such as nylon, polyester, polyethylene, polyamide, polyolefin, polyvinyl alcohol, ethylene vinyl acetate, or polysulfone. In one example, the adsorbent particles immobilized in a matrix are in the form of a sintered medium. While it is understood that the methods and devices described herein encompass removal devices as are known in the art, such methods and devices may be exemplified using the removal device of an amotosalen inactivated platelet product as is commercially available. Such a removal device comprises Hypersol Macronet MN-200 adsorbent contained within a sintered matrix, where the sintered matrix comprises PL2410 plastic as a binder. In one instance, the removal device comprises Hypersol Macronet MN-200 adsorbent in a sintered matrix comprising PL2410, wherein the Hypersol Macronet MN-200 is in an amount of about 5-50 grams, about 5-10 grams, about 10-15 grams, about 15-20 grams, about, 20-25 grams, about 25-30 grams, about 30-35 grams, about 35-40 grams, about 40-45 grams or about 45-50 grams dry weight equivalent.

As various resins may require different processing when used to make the removal devices useful in the methods and devices as described herein, comparison of amounts of adsorbent resins described herein, unless otherwise indicated, are comparison of the dry weight of the resin. For example, the resins are dried to <5% water prior to processing, and the equivalent of the dry weight of adsorbent is used in comparing amounts of resin in use. For example, Hypersol Macronet MN-200 is processed to stabilize the adsorbent, or what is typically referred to as wetting the adsorbent, so as to be directly usable upon contact with a platelet unit. Such a wetted sample may include, for example, about 50% glycerol or other suitable wetting agent. In some embodiments, the adsorbent resin is a polystyrene-divinylbenzene resin. In some embodiments, the polystyrene-divinylbenzene resin is Hypersol Macronet MN-200. In some embodiments, the adsorbent is contained within a sintered matrix, wherein the sintered matrix comprises PL2410 binder. In some embodiments, Hypersol Macronet MN-200 adsorbent is contained within a sintered matrix to provide a removal device.

### Pathogen Inactivation of RBCs

The inactivation of a pathogen in a red blood cell composition is effected by contacting the pathogen in the red blood cell composition with a pathogen-inactivating compound. In any of the embodiments described herein, the pathogen-inactivating compound (e.g., S-303 described herein) may be present in an effective amount (e.g., an effective amount to inactivate a pathogen, such as an amount sufficient to inactivate, for example, at least 1 log, 2 log, 3 log, 4 log or more of a pathogen in the red blood cell composition, if present). Pathogen-inactivating compounds that may be used by the methods of the disclosure include compounds that comprise a functional group which is, or which is capable of forming and has formed, e.g. in situ, a reactive group, such as an electrophilic group. In some cases, the pathogen-inactivating compounds of the present disclosure do not require photoactivation to be reactive. For example, the functional group may be a mustard group, a mustard group intermediate, a mustard group equivalent, an epoxide, a formaldehyde or a formaldehyde synthon. Such functional groups are capable of forming in situ a reactive group, such as an electrophilic aziridine, aziridinium, thiirane or thiiranium ion. A mustard group may be a mono- or bis-(haloethyl)amine group or a mono (haloethyl)sulfide group. A mustard equivalent is a group that reacts by a mechanism similar to the mustards, for example by forming reactive intermediates such as aziridinium and aziridine groups or thiirane and thiiranium groups. Examples include aziridine derivatives, mono or bis-(mesylethyl)amine groups, mono-(mesylethyl)sulfide groups, mono or bis-(tosylethyl)amine groups and mono-(tosylethyl)sulfide groups. A formaldehyde synthon is any compound that breaks down to a formaldehyde, which includes a hydroxylamine such as hydroxymethylglycine. The reactive group of the pathogen-inactivating compound is capable of reacting with the nucleic acids of pathogens, for example with nucleophilic groups on the nucleic acid. The reactive group is also capable of reacting with a nucleophilic group of a quencher. Pathogen-inactivating compounds may also include a component that targets the compound to nucleic acids, such as an anchor portion. The anchor portion comprises a moiety which is capable of binding non-covalently to a nucleic acid biopolymer, such as DNA or RNA, and is also referred to as a nucleic acid binding ligand, nucleic acid binding group, or nucleic acid binding moiety. Examples of such compounds are described in U.S. Pat. Nos. 5,691,132, 6,410,219, 6,136,586, 6,617,157, and 6,709,810, each of which is incorporated by reference herein. Another class of pathogen-inactivating compounds that may be quenched by the methods of the disclosure comprises the above-mentioned reactive groups linked to a nucleic acid binding group via a hydrolysable linker, as described in U.S. Pat. No. 6,514,987, incorporated by reference herein. The anchor portion of the pathogen-inactivating compounds has an affinity for nucleic acids. This affinity may be due to any of several modes of binding to the nucleic acid non-covalently, including, but not limited to, intercalation, minor groove binding, major groove binding, and electrostatic binding (e.g., phosphate backbone binding). The affinity may also be due to mixed modes of binding (e.g., intercalation and minor groove binding). The binding may be sequence-specific (i.e., increased binding affinity for one or more particular nucleic acid sequences over other nucleic acid sequences) or non sequence-specific. Detailed examples of such nucleic acid binding moieties can be found in the above-mentioned patents.

In some embodiments of each of the systems, methods, compositions, processing sets and kits described herein, the pathogen-inactivating compound may comprise a functional group which is, or which forms, a reactive electrophilic group reactive with the nucleophile of the chosen quencher. In some embodiments, the pathogen-inactivating group comprises a nucleic acid binding ligand and a functional group which is, or which forms an electrophilic group. In some embodiments, the PIC comprises a nucleic acid binding ligand that is an intercalator, e.g., an acridine. A specific example of a suitable pathogen-inactivating compound for use in the present invention is β-alanine, N-(acridin-9-yl), 2-[bis(2-chloroethyl)amino]ethyl ester (also alternatively referred to herein as “S-303”), the structure of which is described in the above-referenced patents, including salts thereof.

In some embodiments, the concentration of the pathogen-inactivating compound, such as for example S-303, in the mixture with the red blood cells (e.g., red blood cell composition) is about 0.1 mM to about 10 mM, about 0.1 mM to about 1.0 mM, about 0.2 mM to about 0.5 mM, or 0.2 mM and the quencher is in the range of about 2 mM to 100 mM, about 2 mM to 40 mM, about 5 mM to 40 mM, about 5 mM to 30 mM, or about 10 mM to 30 mM, about 10 mM, about 15 mM, about 20 mM, about 25 mM or about 30 mM. In some embodiments, the concentration of the PIC is about 0.2 mM and the concentration of the quencher is about 20 mM. In some embodiments, the molar ratio of quencher to pathogen inactivation compound once both. components have been mixed with the red blood cell composition is about 10:1 to about 400:1, also about 10:1 to about 200:1, also about 20:1 to about 200:1, also about 50:1 to about 200:1, also about 100:1.

Quenchers for use in methods of the present disclosure are intended to reduce unwanted side-reactions of the reactive electrophilic species used to inactivate pathogens (e.g., binding of the pathogen-inactivating compound to the red blood cell surface which may lead to an undesired immune response). In any of the embodiments described herein, the quencher (e.g., glutathione described herein) may be present in an effective amount (for example, an effective amount to reduce unwanted side reactions, such as the amounts described herein). Suitable quenchers comprise a nucleophilic group that is capable of reacting with the electrophilic group of the pathogen-inactivating compound. Non-limiting examples are described in detail in U.S. Pat. No. 6,709,810, incorporated by reference herein in its entirety. In some embodiments, the quenchers are capable of significantly reducing the unwanted side reactions in a red blood cell composition while allowing the pathogen-inactivating compound to sufficiently inactivate a pathogen that may be contaminating the red blood cell composition. In some embodiments, the improved methods of the present invention provide an effective amount of quencher in combination with an effective amount of pathogen-inactivating compound under conditions which provide optimal reduction in unwanted side reactions combined (e.g., binding of the pathogen-inactivating compound) with sufficient inactivation of pathogens, without significantly altering (e.g., without decreasing) the cell osmotic fragility and without significantly altering (e.g., without increasing) dehydration. A variety of unwanted side reactions may be reduced, such as reaction of the pathogen-inactivating compound with proteins and/or red blood cell components. In some embodiments, the quencher provides optimal reduction in the modification of the red blood cells, such as the binding of IgG to the red blood cells or binding of the pathogen-inactivating compound to the red blood cells. While the methods provided involve the ex vivo treatment of red blood cells (e.g., red blood cell compositions), some quenchers may remain in the composition upon introduction into an individual. As such, in some embodiments, the quenchers of the disclosure are suitable for infusion. Suitable quenchers include, but are not limited to, compounds comprising a thiol group, such as quenchers comprising the amino acid cysteine or a suitable derivative of cysteine, such as N-acetyl cysteine. Examples of such quenchers include cysteine and peptides comprising at least one cysteine, such as glutathione. In some embodiments, the suitable quenchers comprise a derivative of cysteine that can form a thiol group in situ, with or without the use of additional chemicals or added enzymes, such as S-acetyl cysteine or other suitable thiol derived prodrugs of cysteine, or peptides comprising S-acetyl cysteine or other suitable thiol derived prodrugs of cysteine. Suitable derivatives of cysteine are those which either comprise, or are capable of forming in situ, a cysteinyl thiol which is capable of reacting with the electrophilic group of the pathogen-inactivating compound.

In some embodiments, the quencher is a peptide of 2 to 10 amino acids, wherein at least one of the amino acids is cysteine, N-acetyl cysteine, S-acetyl cysteine, or other suitable derivative of cysteine. In some embodiments, the quencher is a peptide of at least 3 amino acids, such as about 3-10 amino acids, also about 3-6 amino acids, wherein at least one of the amino acids is cysteine, N-acetyl cysteine, S-acetyl cysteine, or other suitable derivative of cysteine. In some embodiments, the quencher is a peptide of at least 3 amino acids, such as about 3-10 amino acids, also about 3-6 amino acids, wherein at least one of the amino acids is cysteine, N-acetyl cysteine, S-acetyl cysteine, or other suitable derivative of cysteine, also wherein at least 2 or at least 3 of the amino acids is cysteine, N-acetyl cysteine, S-acetyl cysteine, or other suitable derivative of cysteine.

In a preferred embodiment, the quencher is neutralized glutathione (also known as L-glutathione and γ-L-glutamyl-L-cysteinyl-glycine). Glutathione has many properties that make it particularly useful as a quencher. It is normally present in all cell types. It is not believed to be able to passively penetrate into a pathogen, such as by passing through cell membranes or lipid coats, of bacteria and lipid-enveloped viruses, or by passing through the viral capsid of non-enveloped viruses. At approximately neutral pH glutathione is charged and in the absence of active transport, does not penetrate lipid bilayers to any significant extent. Preferred methods of quenching are provided wherein contamination of a red blood cell composition by a viral or bacterial pathogen is inactivated by at least 2 log, preferably at least 3 log or 4 log or more. At the appropriate conditions, as described by the present disclosure, glutathione is also compatible with in vitro storage of red blood cells and the resulting red blood cell composition is suitable for introduction (e.g., infusion into a subject) in vivo.

In some embodiments, the quencher is glutathione in its reduced form. Glutathione disulfide, the oxidized form of glutathione, may also be used, so long as the glutathione disulfide is sufficiently reduced in solution prior to addition of the solution to the mixture comprising the red blood cells (e.g., red blood cell composition) or sufficiently reduced after addition to the mixture comprising the red blood cells.

In some embodiments, the quencher is a derivative of glutathione, such as a glutathione monoalkyl ester or dialkyl ester, wherein the alkyl group is a straight or branched group having 1 to 10 carbon atoms. Specific examples of alkyl groups include, but are not limited to methyl group, ethyl group, propyl group, isopropyl group, butyl group, isobutyl group, tert-butyl group, pentyl group, isopentyl group, neopentyl group, tert-pentyl group, 1-methylbutyl group, hexyl group, isohexyl group, 2-methylpentyl group, 1-ethylbutyl group, heptyl group, octyl group, nonyl group, and decyl group. For instance, non-limiting examples of glutathione derivatives include glutathione methyl ester, glutathione monoethyl ester, and glutathione monoisopropyl ester. In some embodiments, glutathione oxidized diethyl ester (GSSG-(glycyl)-diethyl-ester) is used. In some embodiments, a thioester of glutathione is hydrolyzed after addition to the red blood cell compositions to form the thiol.

It is understood that in some embodiments, the quencher will be provided in the form of a free acid or base, whereas, in other embodiments, the quencher will be provided in the form of a salt. If the quencher is in the form of a salt, the salt is preferably a pharmaceutically acceptable salt. The pharmaceutically-acceptable salts of compounds (in the form of water- or oil-soluble or dispersible products) include the conventional non-toxic salts or the quaternary ammonium salts which are formed, e.g., from inorganic or organic acids or bases. Examples of such acid addition salts include acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, glucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-napthalensulfonate, nicotinate, oxalate, pamoate, pectinate, persulfate, 3-phenylpropionate, phosphate, picrate, pivalate, propionate, succinate, tartrate, thiocyanate, tosylate, and undecanoate. Base salts include ammonium salts, alkali metal salts such as sodium and potassium salts, alkaline earth metal salts such as calcium and magnesium salts, salts with organic bases such as dicyclohexylamine salts, N-methyl-D-glucamine, and salts with amino acids such as arginine, lysine, and so forth. Also, the basic nitrogen-containing groups may be quaternized with such agents as lower alkyl halides, such as methyl, ethyl, propyl, and butyl chloride, bromides and iodides; dialkyl sulfates like dimethyl, diethyl, didbutyl; and diamyl sulfates, long chain halides such as decyl, lauryl, myristyl and stearyl chlorides, bromides and iodides, aralkyl halides like benzyl and phenethyl bromides and others. Other pharmaceutically acceptable salts include the sulfate salt ethanolate and sulfate salts.

For example, in some embodiments, the quencher is in the form of a pharmaceutically acceptable salt formed from glutathione. In some embodiments, the quencher is in the form of a pharmaceutically acceptable salt formed from glutathione and one or more cations such as sodium, aluminum, calcium, lithium, magnesium, zinc, or tetramethylammonium. In some embodiments, the quencher is glutathione (reduced) and is provided in the form of glutathione monosodium salt (available, e.g., from Biomedica Foscama, Italy). In some other embodiments, the glutathione (reduced) is provided in the form of glutathione hydrochloride salt. In some other embodiments, the glutathione is provided in the form of a glutathione (reduced) disodium salt. In further embodiments, a glutathione monoalkyl ester sulfate is used. In some embodiments, glutathione is provided in the form of glutathione oxidized disodium.

In some embodiments, the concentration of the quencher, such as for example glutathione, in the mixture with the red blood cells (e.g., red blood cell composition) and the pathogen inactivation compound is greater than 2 mM. In some embodiments, the quencher concentration is about 5 mM to about 30 mM. In some embodiments, the quencher concentration is about 15 mM to about 25 mM. In some embodiments, the quencher concentration is about 20 mM.

### Processing Sets, Containers, Kits, and Reusable Consumables

Certain aspects of the present disclosure relate to processing sets. The processing sets of the present disclosure may find use, inter alia, in preparing a pathogen-inactivated blood composition, e.g., as described herein. Any of the exemplary components such as bags and tubings described supra may find use in the processing sets of the present disclosure.

In some embodiments, a mixing container of the present disclosure is part of a processing set, e.g., for processing one or more blood compositions, components, or products, such as platelets, plasma, or RBCs.

In some embodiments, the processing set comprises the mixing container, within which the blood composition in admixture with the determined amount of the PIC can be photochemically inactivated; and at least a first storage container, coupled to the mixing container, wherein the first storage container is configured for storing the pathogen-inactivated blood composition.

Processing sets as described in the present disclosure may involve the use of blood product containers or blood product bag systems, which are well known in the art. In general, such systems may include more than one plastic container, typically plastic bags, where the bags are integrally connected with plastic tubing. Some of the containers described herein include such plastic bags as are known in the storage and handling of blood products, including platelet, plasma, and/or red blood cell products. Blood bags typically can be designed to hold various volumes of fluid, including, but not limited to, volumes ranging from 50 mL to 2 liters, for example having up to a 350 mL capacity, 450 mL capacity, 500 mL capacity, 1 liter capacity, up to a 1.5 liter capacity, or up to a 2 liter capacity. It is understood that when a method refers to a bag, it includes any such plastic bags used in blood product handling. Where such bags are referred to as “mixing bag”, “removal bag”, “product bag”, “storage bag”, or “illumination bag”, it is understood that these bags are typical blood product handling bags, or are similar to such bags in nature. Plastic bags suitable for use according to the present disclosure include for example, those comprising any suitable plastics (e.g., hemocompatible plastics, plastics suitable for blood compositions) known in the art. Plastic bag materials include polyvinyl chloride (PVC), polyolefin, polypropylene, polyethylene, high-density polyethylene (LDPE, LLDPE), polyethylene terephthalate (PET), ethylene vinyl acetate (EVA), ethylene vinyl acetate blended with other plastics, and the like.

In some embodiments, a mixing container of the present disclosure is configured for photochemical inactivation of a blood composition using the determined amount of the PIC under sterile conditions, such as an illumination bag as described herein.

As described herein, where tubing is described as connecting e.g. two bags, such as of a processing set, it is understood that the tubing may be joined at some point therebetween by another component of the connection between the two bags. For example, a removal bag connected to a product bag by a tubing includes wherein the tubing comprises a filter between the two bags, i.e. the tubing is divided by a filter such that fluid flows from one bag to the other through the tubing and filter. In one example, tubing connecting a removal bag and a product bag can include a filter to remove any loose particles from fluid flowing from the removal device to the product bag, i.e. the tubing is divided by, or interrupted by the filter between the bags. Such filters are designed to remove any small particles that may come off of the removal device, while allowing platelets to pass through the filter. The tubing between bags allows for fluid to flow from one bag to another, which can be blocked to prevent the flow until necessary, e.g. as part of the processing the fluid in one bag may be prevented from flowing to the next bag until required for the next step in a process. As such an openable seal, such as a clamp, plug, valve or the like is included in or on the tubing connecting the bags, where the clamp, plug, valve or the like can be selectively opened as required, for example to transfer the fluid from one bag to the next. In certain embodiments, the tubing between bags comprises a breakable seal, such as a breakable valve, whereupon breaking the breakable seal allows for the blood product solution to flow between the bags through the tubing. It is understood that the breakable seal is contained within the connection between containers, such that sterility of the system is maintained. It is also understood that a tubing comprising a filter, or a breakable seal, includes where the tubing may be interrupted by the filter or the seal, for example the tubing runs from one bag and is connected to the filter or seal (an incoming portion of the tubing), and the tubing continues from another portion of the filter or seal to another bag (an outgoing portion of the tubing). In such a configuration, fluid flows from the first bag, through the incoming portion of the tubing, through the filter or seal, and through the outgoing portion of the tubing and into the other bag.

Different containers (e.g., bags) of the systems and methods disclosed in the present application may be used for different purposes, functions and/or steps of a system or process. For example, containers (e.g., bags) may be used to provide solutions for the systems and methods, such as for example pathogen inactivation compound (e.g., PIC container), quencher (e.g., quencher container), diluent (e.g., diluent container), processing solution (PS container), additive solutions (e.g., AS container, storage container). Such containers providing solutions may be of sufficient size and configuration (e.g., ports, connectors, fluid paths (e.g., tubing)) to provide for a single use or transfer of the solution contained therein or more than one (e.g., multiple) use or transfer of the solution contained therein (e.g., multi-use container, bulk container). Such containers for providing solutions may be configured for reconstituting or resuspending a dry form (e.g., lyophilized, freeze-dried, powder) of a compound or substance into a solution, such as for example as a multi-chamber (e.g., dual chamber) container (e.g., bag) configuration, a multi-chamber (e.g., dual chamber) syringe configuration, or a container within a container (e.g., container within a dual chamber container (e.g., dual chamber bag)). Additional non-limiting examples beyond providing solutions may include providing a container configuration for mixing one or more compounds and/or solutions (e.g., mixing container), subjecting one or more compounds and/or solutions to light such as ultraviolet light (e.g., illumination container), incubating one or more compounds and/or solutions (e.g., incubation container), subjecting one or more compounds and/or solutions to a compound removal process, such as for example a compound adsorption device (CAD) for removal of pathogen inactivation compound, for transfer of one or more compounds and/or solutions, and/or for storage, such as for storing a pathogen inactivated blood composition (e.g., storage container). Such containers (e.g., bags) may comprise any material compatible with the contents it is intended to contain, such as for example, medical grade and/or hemocompatible plastics suitable for containing blood compositions of the present disclosure and/or for containing solutions (e.g., processing solution, PIC, quencher, diluent, additive solution) which are intended to be mixed with a blood composition, either directly or indirectly (e.g., after first mixing with one or more other solutions).

Different bags within a blood product bag system can be used for different steps of a process. For example, a system of bags to be used for the pathogen inactivation of a preparation of platelets can include a container with pathogen inactivating compound contained within, a bag for receiving the unit of platelets (e.g., platelet donation) and a pathogen inactivating compound (e.g. an illumination bag), a bag for the illumination of the unit of platelets when the pathogen inactivation method includes illumination (e.g., an illumination bag, and typically the same bag to receive the unit of platelets and pathogen inactivating compound), a bag for the removal of pathogen inactivating compounds and/or by-products thereof from the treated unit of platelets (e.g., referred to as a removal bag, compound adsorption device, CAD), and one or more bags for containing the final platelet product, i.e. the pathogen inactivated platelet unit (e.g., therapeutic dosage unit) that has the concentration of the inactivating compound and/or by-products thereof reduced to below a desired concentration, which is ready for use or can be stored for later use (e.g., referred to as a product bag, storage bag). Each bag in the system is typically made up of a plastic material. For example, the container for containing a solution of pathogen inactivating compound can be made of a suitable plastic such as PL2411 (Baxter Healthcare), or other plastics such as polyvinyl chloride, polyolefins, ethylene vinyl acetate, ethylene vinyl acetate blended with other plastics, and the like. This container is also overwrapped with a material that is impermeable to light of a wavelength that will activate the photoactive pathogen inactivation compound (for example suitable plastic such as PL2420, Baxter Healthcare). The illumination bag for a photoactivated pathogen inactivating compound requires a clear, durable thermoplastic material that is translucent to light of the selected wavelength. Suitable plastics that are translucent to light in the UVA wavelength range include polyvinyl chloride, polyolefins, ethylene vinyl acetate, ethylene vinyl acetate blended with other plastics, or other blends of thermoplastic polymers. Such suitable plastics include PL2410 (Baxter Healthcare) and PL732 (Baxter Healthcare). Similar materials may be used to make the removal bag and the product bag. The product bags include, for example, those made of PL2410. Suitable bag materials are discussed, for example, in PCT publication number WO 2003078023, and U.S. Pat. No. 7,025,877, the disclosures of which are hereby incorporated by reference as it relates to such bag materials and related materials. In all cases, the materials used in preparing the processing set have to be sterilizable by known methods such as steam and gamma or electron beam radiation used to ensure sterility of the processing set. While these are exemplary materials for making the bags, the methods described herein are applicable to processes using any suitable bag material as would be readily available to one skilled in the art, and can also be used with containers other than bags. The bags used for illumination, removal, and storage are also designed to allow for gases such as oxygen and carbon dioxide to go into and out of the blood bag, so that the platelets therein have adequate oxygen supply and carbon dioxide levels during the processing and storage.

In some embodiments, the methods and processing sets of the present disclosure do not include and/or require use of a compound adsorption device (CAD). In other embodiments, the methods and processing sets of the present disclosure can include use of a CAD. CADs are known as a type of removal device for reducing the concentration of pathogen inactivating compound, such as a small organic compound, e.g. platelet inactivating compound, and by-products thereof in a preparation of platelets, while substantially maintaining a desired biological activity of the platelets. In some embodiments, the CAD container may contain one or more materials, such as for example, adsorbent particles (e.g., adsorbent beads), and be suitable for also containing a preparation of platelets from which the concentration of pathogen inactivating compound and by-products thereof are to be reduced. Such a removal device is generally intended to be used in a batch mode, i.e. the device is placed in contact with the platelets, and continued contact with the removal device, e.g. with shaking to allow essentially the entirety of the solution of platelets to come into contact with the removal device over time of contact, results in reducing the levels of pathogen inactivating compound. Such batch devices entail the use of an adsorbent particle that binds the pathogen inactivation compound, and can be used by either adding adsorbent particles directly to the platelet container (e.g., bag) following illumination or transferring the platelets to a bag containing the adsorbent particles following illumination and the platelets are then agitated for a specified period of time with the platelet preparations contacting the removal device. While free adsorbent particles may be used as a removal device, such particles may be contained within a mesh pouch, such as a polyester or nylon mesh pouch, which allows for contact of the platelet solution with the adsorbent particles while containing the particles within the pouch. Alternatively, the adsorbent particles may be immobilized within a matrix, where the immobilized matrix can reside directly in the blood bag used for batch removal, or may be similarly contained within a mesh pouch. In some instances, the removal device comprises porous adsorbent particles in an amount sufficient to reduce the pathogen inactivating compound to below a desired concentration, wherein the adsorbent particles have an affinity for the pathogen inactivating compound, where it is understood such adsorbent particle can be selected to best adsorb the compound or compounds to be removed, with minimal effect on components that should not be removed or damaged by contact with the adsorbent particle. A variety of adsorbent particles are known, including generally particles made from any natural or synthetic material capable of interacting with compounds to be removed, including particulates made of natural materials such as activated carbon, silica, diatomaceous earth, and cellulose, and synthetic materials such as hydrophobic resins, hydrophilic resins or ion exchange resins. Such synthetic resins include, for example, carbonaceous materials, polystyrene, polyacrylic, polyacrylic ester, cation exchange resin, and polystyrene-divinylbenzene. Detailed description of such removal devices suitable for use in the methods as described herein can be found in PCT publication numbers WO 1996040857, WO 1998030327, WO 1999034914, and WO 2003078023, the disclosures of which are hereby incorporated by reference with respect to the discussion of such removal devices and the adsorbent particles and other materials used to prepare such devices. Exemplary adsorbent particles include, but are not limited to, Amberlite (Rohm and Haas) XAD-2, XAD-4, XAD-7, XAD-16, XAD-18, XAD-1180, XAD-1600, XAD-2000, XAD-2010; Amberchrom (Toso Haas) CG-71m, CG-71c, CG-161m, CG161c; Diaion Sepabeads (Mitsubishi Chemicals) HP20, SP206, SP207, SP850, HP2MG, HP20SS, SP20MS; Dowex (Dow Chemical) XUS-40285, XUS-40323, XUS-43493 (also referred to as Optipore V493 (dry form) or Optipore L493 (hydrated form)), Optipore V503, Optipore SD-2; Hypersol Macronet (Purolite) MN-100, MN-102, MN-150, MN-152, MN-170, MN-200, MN-202, MN-250, MN-252, MN-270, MN-300, MN-400, MN-500, MN-502, Purosorb (Purolite) PAD 350, PAD 400, PAD 428, PAD 500, PAD 550, PAD 600, PAD 700, PAD 900, and PAD 950. The material used to form the immobilized matrix comprises a low melting polymer, such as nylon, polyester, polyethylene, polyamide, polyolefin, polyvinyl alcohol, ethylene vinyl acetate, or polysulfone. In one example, the adsorbent particles immobilized in a matrix are in the form of a sintered medium. While it is understood that the methods and devices described herein encompass removal devices as are known in the art, such methods and devices may be exemplified using the removal device of an amotosalen inactivated platelet product as is commercially available. Such a removal device comprises Hypersol Macronet MN-200 adsorbent contained within a sintered matrix, where the sintered matrix comprises PL2410 plastic as a binder. In one instance, the removal device comprises Hypersol Macronet MN-200 adsorbent in a sintered matrix comprising PL2410, wherein the Hypersol Macronet MN-200 is in an amount of about 3-50 grams, about 3-40 grams, about 3-30 grams, about 3-20 grams, about 3-7 grams, about 7-15 grams, about 10-20 grams, about 5-50 grams, about 5-10 grams, about 10-15 grams, about 15-20 grams, about, 20-25 grams, about 25-30 grams, about 30-35 grams, about 35-40 grams, about 40-45 grams or about 45-50 grams dry weight equivalent.

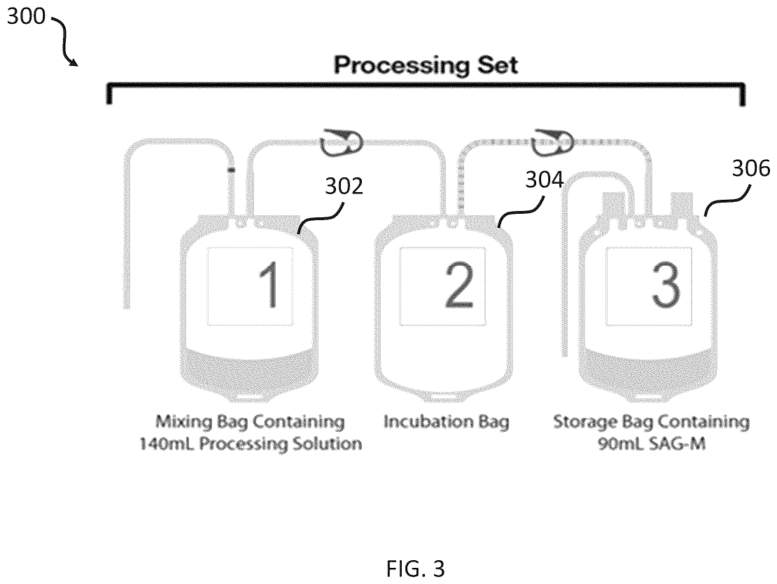

In some embodiments, the processing set comprises a mixing container, a second container coupled to the mixing container, which second container is configured for incubating the blood composition and the determined amount of the PIC (and optionally, quencher); and at least a first storage container, coupled to the second container, wherein the first storage container is configured for storing the pathogen-inactivated blood composition. An exemplary processing set 300 for processing a RBC composition is shown in FIG. 3. Processing set 300 includes mixing container 302, incubation container (e.g., bag) 304, and storage container 306. Mixing container 302 and incubation container (e.g., bag) 304 are connected via sterile tubing (which has a clamp affixed), and incubation container (e.g., bag) 304 and storage container 306 are also connected via sterile tubing (which has a removable clamp affixed). In some embodiments, mixing container 302 contains a processing solution into which quencher (e.g., GSH), the RBC composition and the PIC (e.g., amustaline) are transferred. In some embodiments, storage container 306 contains a RBC additive solution (e.g., SAG-M), for exchange and storage of the pathogen-inactivated RBC.

Generally, processing sets for RBCs can comprise suitable containers, tubing and solution(s) for introducing, mixing and/or incubating the input red blood cells, pathogen-inactivating compound, quencher and processing solution, as well as replacing the solution used during the pathogen-inactivation treatment with a red blood cell additive solution, and separating the treated red blood cells (e.g., pathogen-inactivated red blood cell preparations) into one or more (e.g., at least two) units of pathogen-inactivated red blood cells suitable for infusion into a subject. In some embodiments, a processing set is provided, comprising a) a first container (e.g., bag) suitable for receiving and mixing under sterile conditions (e.g., aseptic conditions) the red blood cells, the pathogen-inactivating compound and the quencher, wherein the first container contains a processing solution as described herein; b) a second container suitable for incubating the mixture of red blood cells, pathogen-inactivating compound, quencher and processing solution, wherein the second container is coupled (e.g., connected) to the first container such that the mixture can be transferred from the first container to the second container under sterile conditions; c) a third container suitable for replacing the solution use during the pathogen-inactivation treatment and storage of the pathogen-inactivated red blood cells, wherein the third container contains a red blood cell additive solution as described herein and is coupled (e.g., connected) to the second container such that the treated red blood cells can be transferred from the second container to the third container under sterile conditions; and optionally, d) a fourth container suitable for storage of the pathogen-inactivated red blood cells, wherein the fourth container is or may be coupled (e.g., connected) to the third container such that the pathogen-inactivated red blood cells can be transferred from the third container to the fourth container under sterile conditions. In some embodiment, the processing sets and/or kits may further comprise one or more additional containers that contain the pathogen-inactivating compound and/or quencher, wherein the additional containers are coupled or are configured to be coupled (e.g., connected) to the first container, such that the contents of the additional container(s) can be transferred to the first container under sterile conditions. The containers as described in the processing sets and kits provided herein may be made of any material suitable and/or known in the art for processing (e.g., pathogen inactivation) and storage of red blood cells. The containers (e.g., storage containers) should allow maintenance of sterile (e.g., aseptic) conditions for its contents. The kits provided herein may further contain additive solutions, buffers, or other solutions that may be used in carrying out any of the methods provided herein.

In some embodiments, a processing set for use with a device of the present disclosure is provided, the processing set comprising a mixing container (e.g., illumination container), wherein the mixing container is coupled (e.g., by way of a first port of the mixing container) to a fluid path (e.g., sterile tubing), wherein the fluid path comprises 1) a first segment extending from the mixing container to a junction, 2) a junction (e.g., Y-junction) connected to the first segment, 3) a second segment connected to the junction, wherein the second segment is coupled or configured to be coupled (e.g., reversibly coupled, sterile coupled) to a container containing a PIC, and 4) a third segment connected to the junction, wherein the third segment is configured to be coupled (e.g., reversibly coupled, sterile coupled) to a container containing a biological fluid (e.g., blood composition). In some embodiments, each of the first, second and third segments of the fluid path comprise tubing (e.g., sterile tubing). In some embodiments, the mixing container is connected (e.g., by way of sterile tubing) to one or more storage containers, the storage container(s) configured to store a pathogen inactivated fluid. In some embodiments, the mixing container is connected (e.g., by way of sterile tubing) to a container containing a compound adsorption device (CAD container). In some embodiments, the CAD container is connected (e.g., by way of sterile tubing) to one or more storage containers, the storage container(s) configured to store a pathogen inactivated fluid.

### Devices and Systems

Certain aspects of the present disclosure provide devices, reusable consumables and systems for use in preparing a pathogen inactivated biological fluid, such as, for example, a blood composition. In some embodiments, the devices comprise one or more components configured to control flow of one or more fluids (e.g., through or within a fluid path). In some embodiments, the systems comprise a fluid path, such as for example, sterile tubing, configured to be removably mounted to the device. In some embodiments, the fluid path is part of a reusable consumable of the system, wherein the reusable consumable comprises one or more fluid paths, one or more connectors, one or more manifolds, and/or one or more pumps. In some embodiments, the fluid path comprises a manifold and/or a pump. In some embodiments, the fluid path may provide for flow of fluid to a manifold and/or pump, such as for example, from container (e.g., reversibly coupled container, multi-dose “bulk” container, via a connector). In some embodiments, the fluid path may provide for flow of fluid from a manifold and/or pump, such as for example, to a processing set (e.g., reversibly coupled processing set, via a connector). In some embodiments, the fluid path may comprise more than one portion or segment of fluid path, such as for example a first portion or segment providing for flow of fluid to a manifold and/or pump (e.g., from a container) and a second portion or segment providing for flow of fluid from a manifold and/or pump (e.g., to a processing set). In some embodiments, the fluid path is removably mounted to or by way of one or more pumps, manifolds, clamps, valves, fasteners, fluid connections/connectors, and/or sensors of the device. In some embodiments, the fluid path is configured to be coupled (e.g., sterile coupled, reversibly coupled) to one or more of 1) a container containing a pathogen inactivation compound (PIC), 2) a container containing a diluent or additive solution, 3) a container containing a quencher, and/or 4) a container containing a processing solution. In some embodiments, the fluid path is configured to be coupled (e.g., sterile coupled, reversibly coupled) to a processing set, such as for example a pathogen inactivation processing set (e.g., INTERCEPT Blood System, Cerus Corporation). In some embodiments, the fluid path is configured to be coupled (e.g., sterile coupled, reversibly coupled) to a mixing container (e.g., of a processing set of the present disclosure). Coupling may include, for example, coupling using a sterile tubing welder or a reversible coupling or connector, such as for example a Luer connector, closed system transfer device (CSTD), neutral displacement connector, valve connector, needle free activation connector, actuator connector, piston connector, twist connector, spike port connector, arm lock connector, USP800 connector, USP797 connector, and/or K-Zero connector. In some embodiments, the systems and methods may comprise a vial adapter. In some embodiments, the systems and methods of the present disclosure comprise connector(s) configured for multiple uses (e.g., coupling, uncoupling), such as for example at least 5 times, at least 10 times, at least 25 times, at least 50 times, at least 100 times, at least 200 or more times. In some embodiments, CSTD type connectors may be preferable, such as for example, connectors used in the transfer of potent compounds (e.g., highly potent compounds, hazardous solutions, certain PICs), to provide an additional level of containment (e.g., when coupling and/or uncoupling). CSTD connectors are well known in the art and any CSTD connector may be used in the systems and methods disclosed herein. In some embodiment, a disposable consumable component of the system may comprise one or more connectors. In some embodiments, a processing set may comprise one or more connectors. The present application contemplates that connector(s) as used in the systems and methods herein may comprise more than one portion (e.g., to be coupled), with a fluid path (e.g., of a consumable of the system) comprising one portion of a connector and a processing set (e.g., fluid path extending therefrom) or container (e.g., fluid path extending therefrom) comprising the other portion of the connector, so as to provide for reversibly coupling. In some embodiments, the systems comprise a device comprising one or more components configured to control flow of one or more fluids, and a fluid path (e.g., tubing, sterile tubing) configured to be removably mounted to the device. In some embodiments, the systems comprise a fluid path comprising tubing, (e.g., sterile tubing); a device comprising one or more components configured to control flow of fluids through the fluid path (e.g., fluid path configured to be removably mounted to the device); and a processing set of the present disclosure. In some embodiments, a processing set as provided herein is configured to be reversibly coupled to the fluid path. In some embodiments, the fluid path is configured to be mounted on the device. In some embodiments, component(s) of the system, such as for example a container, a fluid path and/or a processing set, may further comprise one or more filters (e.g., 0.2 μM filter) upstream and/or downstream of a connector or other point of coupling (e.g., to further ensure sterility of the transferred fluid).

For example, in some embodiments, the processing set comprises: (i) a mixing container for containing a blood composition in admixture with the PIC, wherein the mixing container is configured to be coupled (e.g., sterile coupled, reversibly coupled) to the fluid path; and (ii) at least a first storage container coupled to the mixing container, wherein the first storage container is configured for storing the pathogen-inactivated blood composition. In some embodiments, the processing set comprises: (i) a mixing container for containing a blood composition in admixture with the PIC, wherein the mixing container is configured to be coupled (e.g., sterile coupled, reversibly coupled) to the fluid path; (ii) a second container, coupled to the mixing container, wherein the second container is configured for incubating a blood composition and the PIC; and (iii) at least a first storage container, coupled to the second container, wherein the first storage container is configured for storing the pathogen-inactivated blood composition. In some embodiments, the processing set comprises: (i) a mixing container for containing (e.g., mixing) a blood composition in admixture with the PIC (e.g., mixing container configured for photochemical inactivation of the blood composition in admixture with the PIC), wherein the mixing container is configured to be coupled (e.g., sterile coupled, reversibly coupled) to the fluid path; (ii) a second container, coupled to the mixing container, wherein the second container contains a compound adsorption device (CAD); and (iii) at least a first storage container, coupled to the second container, wherein the first storage container is configured for storing the pathogen-inactivated blood composition. In some embodiments, the processing set comprises: (i) a mixing container for containing a blood composition in admixture with the PIC, wherein the mixing container is configured to be coupled (e.g., sterile coupled, reversibly coupled) to the fluid path; (ii) a second container, coupled to the mixing container, wherein the second container is configured for photochemical inactivation of a blood composition in admixture with the PIC; and (iii) at least a first storage container, coupled to the second container, wherein the first storage container is configured for storing the pathogen-inactivated blood composition.

In some embodiments, the PIC container and the mixing container are configured to be coupled (e.g., sterile coupled) to the fluid path. In some embodiments, the processing set and/or the device is/are reversibly coupled to the fluid path. In some embodiments, a reversible coupling of the present disclosure is a sterile, reversible coupling, e.g., using a closed system transfer device (CSTD), neutral displacement connector, or K-Zero connector (Fresenius-Kabi).

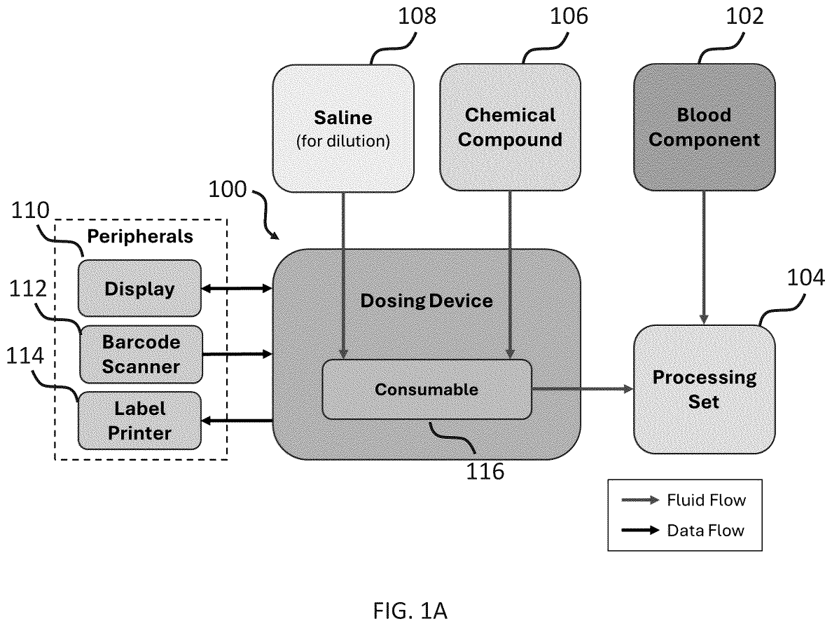

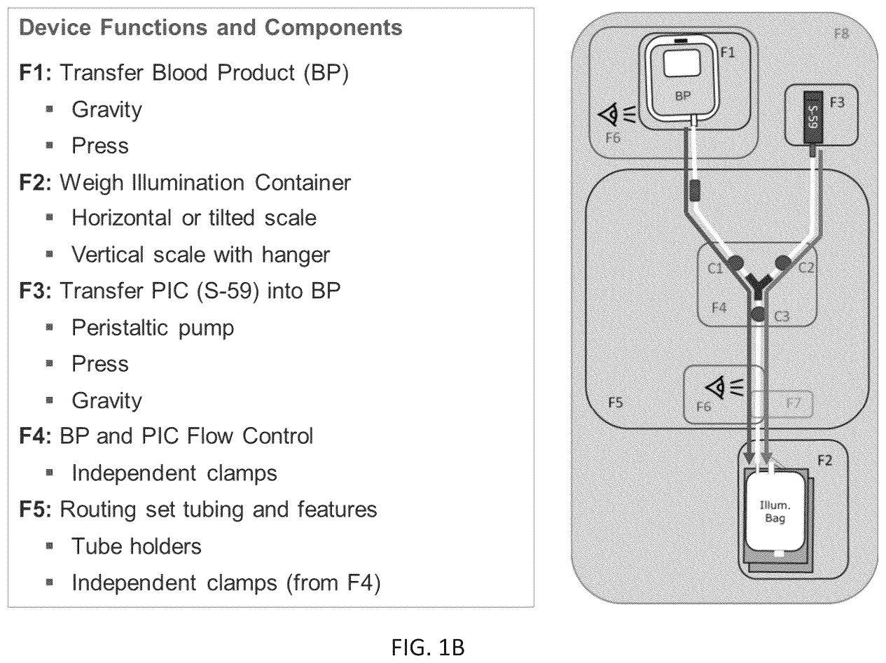

An exemplary and non-limiting system and device of the present disclosure is shown as device 100 in FIG. 1A. Device 100 is used for pathogen inactivation of a blood composition supplied in blood composition container 102. The blood composition (e.g., blood component) from blood composition container 102 is transferred into a mixing container 104, which can be part of a processing set (e.g., as described herein). Device 100 can determine the amount of PIC (referred to in FIG. 1A as chemical compound, supplied in PIC container 106) to pathogen-inactivate the blood composition (e.g., inactivate a pathogen in the blood composition). The amount of PIC may be determined by any of the methods and systems described herein. The determined amount of PIC is transferred from PIC container 106 into mixing container 104 via a first fluid path, which can include or comprise consumables 116. For example, for transfer of PIC, a PIC container containing sufficient volume of PIC for treating multiple units of blood composition is reversibly coupled to the first fluid path of the system. A mixing container (e.g., container of the processing set) is also reversibly coupled to the first fluid path. If necessary, a diluent (e.g., as described herein) is supplied in diluent container 108. In some embodiments, an amount of diluent can be transferred from diluent container 108 into PIC container 106, then into mixing container 104. In some embodiments, an amount of PIC can be transferred from PIC container 106 into diluent container 108, then into mixing container 104. In some embodiments, an amount of diluent from diluent container 108 can be transferred into another container (e.g., part of a fluid path, consumable), along with an amount of PIC from PIC container 106, then the mixture of diluent and PIC (e.g., diluted PIC) is transferred into mixing container 104. Device 100 can also be connected to peripherals such as display 110 (for displaying information and optionally receiving a user input via touchscreen), barcode scanner 112 (for receiving barcode inputs), and label printer 114 (for printing labels, e.g., for blood bags or components of the processing set).

Another exemplary and non-limiting system and device of the present disclosure is shown in FIG. 1B. A system is provided for preparing a pathogen inactivated blood composition, comprising: (a) a device comprising one or more components configured to control flow of fluids through a fluid path, and (b) a processing set, wherein the processing set, each shown in FIG. 1B. The processing set comprises a mixing container, the mixing container connected to a fluid path, wherein the fluid path comprises: (i) a first segment extending from the mixing container to a junction, (ii) a junction (e.g., Y-junction) connected to the first segment, (iii) a second segment connected to the junction, wherein the second segment is coupled or configured to be coupled to a container containing a PIC, and (iv) a third segment connected to the junction, wherein the third segment is configured to be coupled to a container containing a blood composition. The blood composition (BP) from blood composition container is transferred into a mixing container configured for photochemical inactivation of a blood composition in admixture with a PIC (e.g., illumination bag), which is part of the processing set. The transfer of BP is by way of the fluid path (e.g., sterile tubing) first segment and third segments and junction (e.g., as described herein). Flow of blood composition may be via gravity flow and/or assisted/controlled by the device (e.g., press, pump). The device can determine the amount of PIC (e.g., S-59) required for pathogen inactivation of the blood component by any of the methods and systems described herein. PIC is supplied from PIC container by way of the fluid path (e.g., sterile tubing) first segment and second segments and junction (e.g., as described herein), with dose and flow controlled by the device. In some embodiments, a PIC container containing sufficient volume of PIC for treating multiple units of blood composition may be reversibly coupled to the fluid path of the processing set for each unit of blood composition to be treated. In some embodiments, the mixing container (e.g., illumination bag) is connected to one or more storage containers, the storage container(s) configured to store a pathogen inactivated fluid. In some embodiments, the mixing container is connected to a container containing a compound adsorption device (CAD container). In some embodiments, the CAD container is connected to one or more storage containers, the storage container(s) configured to store a pathogen inactivated fluid.

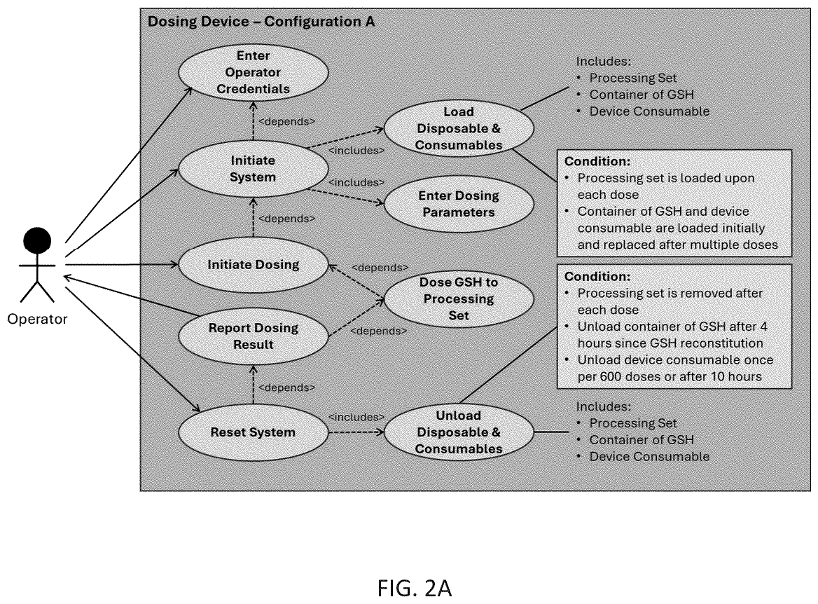

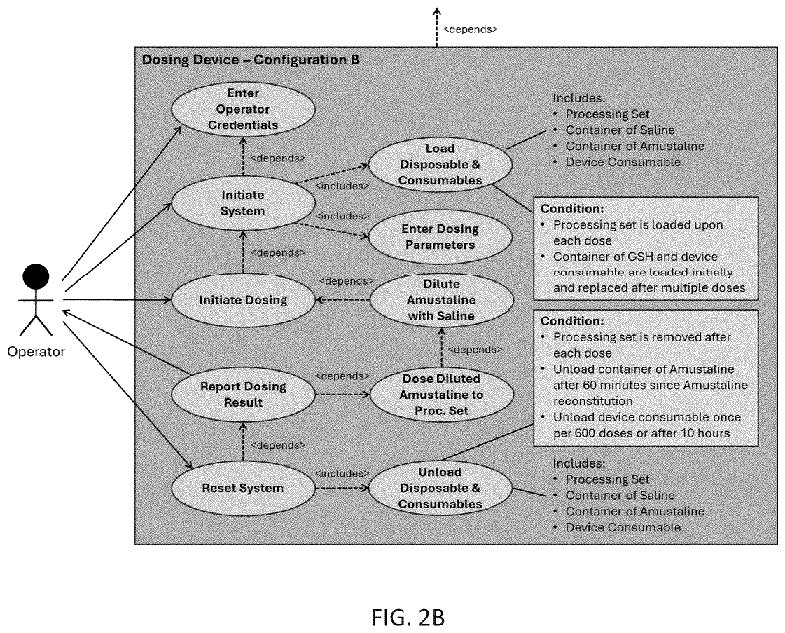

Exemplary and non-limiting procedures for using a device of the present disclosure (e.g., device 100) to pathogen-inactivate a blood composition are illustrated in FIGS. 2A & 2B. For example, FIG. 2A illustrates a procedure for using a device of the present disclosure to dose a quencher (e.g., GSH) to a blood composition. A similar procedure may be used to dose a PIC (e.g., amustaline) to the blood composition, such as for example dosing the PIC after dosing the quencher. In the example of FIG. 2A, an operator enters credentials to the device to initiate the system. The operator also loads (e.g., mounts) the processing set and consumables, the consumables comprising any required containers (e.g., quencher (e.g., GSH) container, PIC (e.g., amustaline) container, diluent or additive container) and fluid path(s). In some embodiments, the loading step also includes coupling of the container(s) and processing set (e.g., mixing container of processing set) to a fluid path of the consumables. The transfer of blood component from a blood component container to the processing set (e.g., mixing container of processing set) is performed (e.g., as disclosed herein), before, during or after coupling of the processing set (e.g., mixing container) to the fluid path. The dosing parameters are entered, such as for example, by any one or more of user input, scanning of information from the blood composition container, and/or determining dosing parameters by the device (e.g., based on weight, volume). Dosing is next initiated and the transfer of GSH (or PIC, in the case of PIC dosing) to the processing set (e.g., mixing container) occurs. Any dosing results and/or reports may be provided by the device. The processing set is uncoupled from the system (e.g., from the fluid path) after dosing of each unit. The consumables may be unloaded from the system as needed, such as for example, after a predefined period of time (e.g., post-reconstitution) and/or after delivery of a predetermined number of doses. The system is reset for processing of the next blood component. In the event of dosing both a quencher and a PIC, this procedure may be carried out sequentially using the same system/device, or the quencher may be dosed using a first system/device followed by uncoupling of the processing set and using a second system/device for the dosing of PIC. Such a sequential procedure using multiple systems/devices may provide for improved (e.g., higher throughput), such as for example, providing for processing of greater numbers of blood composition units within a period of time (e.g., prior to expiry of a reconstituted PIC and/or quencher solution).

In another example, FIG. 2B illustrates a procedure for using a device of the present disclosure to dose a PIC (e.g., amustaline) to a blood composition, including an additional step of diluting a more concentrated solution of PIC prior to dosing. A similar procedure may be used to dose a quencher (e.g., GSH) to the blood composition, such as for example dosing the quencher before dosing the PIC. In the example of FIG. 2B, an operator enters credentials to the device to initiate the system. The operator also loads (e.g., mounts) the processing set and consumables, the consumables comprising any required containers (e.g., PIC (e.g., amustaline) container, quencher (e.g., GSH) container, diluent or additive container) and fluid path(s). In some embodiments, the loading step also includes coupling of the container(s) and processing set (e.g., mixing container of processing set) to a fluid path of the consumables. The transfer of blood component from a blood component container to the processing set (e.g., mixing container of processing set) is performed (e.g., as disclosed herein), before, during or after coupling of the processing set (e.g., mixing container) to the fluid path. The dosing parameters are entered, such as for example, by any one or more of user input, scanning of information from the blood composition container, and/or determining dosing parameters by the device (e.g., based on weight, volume). The dosing parameters may also include, for example, any dilution amounts or volumes. Dosing is next initiated, and the PIC is diluted with a diluent, such as for example, saline, followed by the transfer of the diluted PIC (or diluted GSH, in the case of GSH dosing) to the processing set (e.g., mixing container) occurs. Any dosing results and/or reports may be provided by the device. The processing set is uncoupled from the system (e.g., from the fluid path) after dosing of each unit. The consumables may be unloaded from the system as needed, such as for example, after a predefined period of time (e.g., post-reconstitution) and/or after delivery of a predetermined number of doses. The system is reset for processing of the next blood component. In the event of dosing both a PIC and a quencher, this procedure may be carried out sequentially using the same system/device, or the quencher may be dosed using a first system/device followed by uncoupling of the processing set and using a second system/device for the dosing of PIC. Such a sequential procedure using multiple systems/devices may provide for improved (e.g., higher throughput), such as for example, providing for processing of greater numbers of blood composition units within a period of time (e.g., prior to expiry of a reconstituted PIC and/or quencher solution).

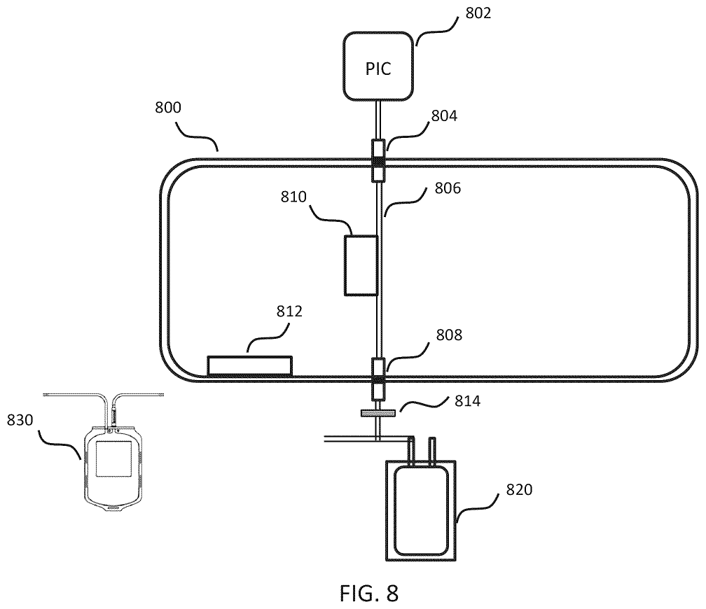

FIG. 8 depicts an exemplary system (e.g., dosing device system) for preparing a pathogen-inactivated blood composition, in accordance with some embodiments. The depicted system includes dosing device 800 (e.g., a device comprising one or more components configured to control flow of one or more fluids of the present disclosure), one or more device-associated consumable components (e.g., reusable consumables) such as for example fluid path 806, connectors 804 and/or 808 and optional pump 810, and processing set 820 (e.g., a processing set of the present disclosure). In some embodiments, connectors 804 and/or 808 may be a portion of a connector, with the other portion of the connector a part of processing set 820 and/or container 802 (e.g., each a portion that is configured to join with the other portion). Device 800 comprises pump 810 and/or a controller and/or actuator of pump 810, and optional scale 812. In this example, PIC container 802 is coupled (e.g., reversibly, sterile coupled) to fluid path 806 via connector 804, which connects to fluid path 806 and connector 808 downstream. Pump 810 may be connected to or otherwise associated with (e.g., loaded, mounted, attached, coupled) fluid path 806 and controls flow of fluid (e.g., comprising a PIC of the present disclosure) through fluid path 806, e.g., from PIC container 802 through connector 804 into fluid path 806, and subsequently through connector 808 into processing set 820 optionally through filter 814. Processing set 820 may be connected to connector 808 via optional filter 814. Alternatively, or in addition, processing set 820 may comprise a portion of fluid path (e.g., tubing) which comprises optional filter 814, and the fluid path may be connected to connector 808. This allows for transferring a determined amount of PIC from the PIC container into one or more containers of processing set 820 in a sterile manner. Connectors 804 and 808 can be the same type of connector of the present disclosure, or different types of connectors of the present disclosure. In some embodiments, device 800 comprises a housing. Optional scale 812 can be within or outside of the housing. Scale 812 can be used to weigh a blood composition of the present disclosure.

The system and/or device may further comprise control circuitry and a computer system (not depicted). In some embodiments control and/or adjustment of various parameters of pump 810 may be performed by a control circuitry operatively coupled (e.g., communicatively coupled) to the pump and/or to a controller or actuator of the pump, to various sensors, to a manifold (e.g., valve/manifold) and/or to a controller or actuator of the manifold, and/or to a computer system. As used herein, “operatively coupled” refers to any wired or wireless connection between two or more components that enables the two or more components to exchange information, control instructions, and/or control signals. As will be discussed in more detail below, control circuitry may receive control instructions and/or control signals from a computer system and send control instructions and/or control signals to various components of the systems and/or device to adjust or set various parameters associated with various components of the system and/or device, such as for example pump 810 and/or a manifold. Adjustment of various parameters may be desirable to ensure that pump parameters are in accordance with the dosing of one or more solutions to the blood composition. It should be recognized that, in some examples, control circuitry and/or the function of control circuitry may be included within a computer system. In some examples, control circuitry may include a computer system and/or the function of a computer system. In some examples, control circuitry may be structurally attached to device 800 and/or pump 810 (and/or a controller or actuator of pump 810), a manifold (and/or a controller or actuator of a manifold), and/or one or more sensors. In some examples, control circuitry may be integrated with device 800 and/or pump 810 and/or a manifold. A computer system may be operatively coupled (wired or wirelessly) to control circuitry and/or to pump 810 (and/or a controller or actuator of pump 810), a manifold (and/or a controller or actuator of a manifold), and/or one or more sensors. A computer system may include one or more processors, memory, an input/output (I/O) interface, and a user interface. One or more processors may be one or more of any type of general purpose computer processor. Memory, or computer readable medium may include one or more of readily available memory such as random access memory (RAM), read-only memory (ROM), hard disk, optical storage media (e.g., compact disc or digital video disc), flash drive, or any other form of digital storage, local or remote. In some examples, a non-transitory computer-readable storage medium of memory may be used to store instructions for transfer of one or more solutions in accordance with the dosing requirements for a blood composition, as will be discussed herein. A computer system may encompass any variety of computers, such as a personal computer (PC), a desktop computer, a laptop, a computer terminal, a server computer, a tablet computer, a smartphone, a personal digital assistant (PDA), etc. In some examples, control circuitry and/or the function of control circuitry may be included within computer system.

Blood composition container 830 comprises a blood composition of the present disclosure. Blood composition container 830 can be coupled (e.g., sterile coupled) to processing set 820, allowing for the blood composition to be transferred into processing set 820 (e.g., before, during, or after coupling processing set 820 to fluid path 806 and/or transfer of the PIC into processing set 820). In some embodiments, the blood composition is a platelet and/or plasma composition. In some embodiments, the PIC is a photoactive pathogen inactivating compound of the present disclosure, e.g., a psoralen such as amotosalen. In some embodiments, the blood composition comprises RBCs (e.g., an RBC or whole blood composition). In some embodiments, the PIC comprises a nucleic acid binding ligand of the present disclosure that is an intercalator, e.g., an acridine such as β-alanine, N-(acridin-9-yl), 2-[bis(2-chloroethyl)amino]ethyl ester.

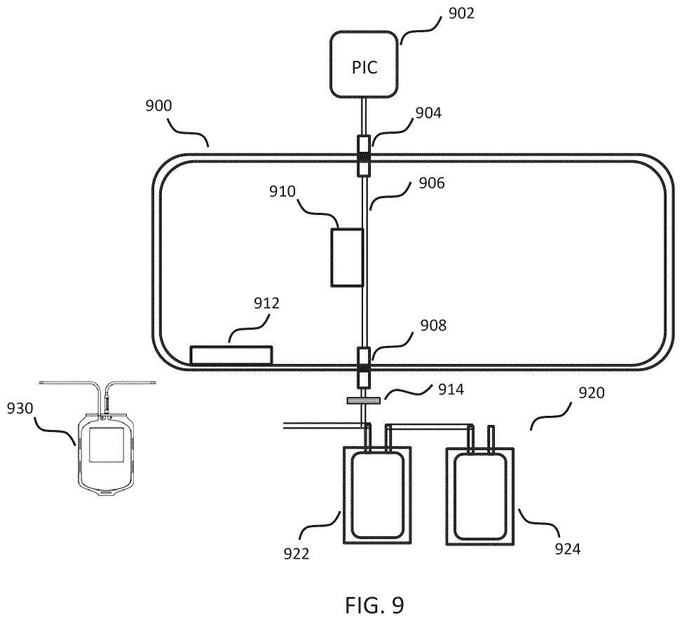

FIG. 9 depicts an exemplary system (e.g., dosing device system) for preparing a pathogen-inactivated blood composition, in accordance with some embodiments. The depicted system includes dosing device 900 (e.g., a device comprising one or more components configured to control flow of one or more fluids of the present disclosure), one or more device-associated consumable components (e.g., reusable consumables) such as for example fluid path 906, connectors 904 and/or 908 (or portion thereof) and optional pump 910, and processing set 920 (e.g., a processing set of the present disclosure). In some embodiments, connectors 904 and/or 908 may be a portion of a connector, with the other portion of the connector a part of processing set 920 and/or container 902 (e.g., each a portion that is configured to join with the other portion). Device 900 comprises pump 910 and/or a controller and/or actuator of pump 910, and optional scale 912. In this example, PIC container 902 is coupled (e.g., reversibly, sterile coupled) to to fluid path 906 via connector 904, which connects to fluid path 906 and connector 908 downstream. Pump 910 may be connected to or otherwise associated with (e.g., loaded, mounted, attached, coupled) fluid path 906 and controls flow of fluid (e.g., comprising a PIC of the present disclosure) through fluid path 906, e.g., from PIC container 902 through connector 904 into fluid path 906, and subsequently through connector 908 into processing set 920 (specifically, into mixing and/or illumination container 922) optionally through filter 914. Processing set 920 may be connected to connector 908 via optional filter 914. Alternatively, or in addition, processing set 920 may comprise a portion of fluid path (e.g., tubing) which comprises optional filter 914, and the fluid path may be connected to connector 908. This allows for transferring a determined amount of PIC from the PIC container into one or more containers of processing set 920 in a sterile manner. In this example, processing set 920 comprises mixing and/or illumination container 922 and storage container 924. Connectors 904 and 908 can be the same type of connector of the present disclosure, or different types of connectors of the present disclosure. In some embodiments, device 900 comprises a housing. Optional scale 912 can be within or outside of the housing. Scale 912 can be used to weigh a blood composition of the present disclosure. In some embodiments, the system and/or device of FIG. 9 may comprise control circuitry and a computer system (not depicted) substantially similar to the system and/or device described for FIG. 8.

Blood composition container 930 comprises a blood composition of the present disclosure. Blood composition container 930 can be coupled (e.g., sterile coupled) to processing set 920, allowing for the blood composition to be transferred into processing set 920 (e.g., before, during, or after coupling processing set 920 to fluid path 906 and/or transfer of the PIC into processing set 920). In some embodiments, the blood composition is a platelet and/or plasma composition. In some embodiments, the PIC is a photoactive pathogen inactivating compound of the present disclosure, e.g., a psoralen such as amotosalen. Processing set 920 includes mixing and/or illumination container 922 and storage container 924. After transfer of the blood composition from container 930 into container 922, and the PIC from container 902 into container 922 via device 900, the PIC can be admixed with the blood composition and/or subjected to illumination (e.g., for photochemical pathogen inactivation) in container 922. Following illumination, the pathogen-inactivated blood composition can be transferred into storage container 924, e.g., for storage. In some embodiments, the blood composition comprises RBCs (e.g., an RBC or whole blood composition). In some embodiments, the PIC comprises a nucleic acid binding ligand of the present disclosure that is an intercalator, e.g., an acridine such as β-alanine, N-(acridin-9-yl), 2-[bis(2-chloroethyl)amino]ethyl ester.

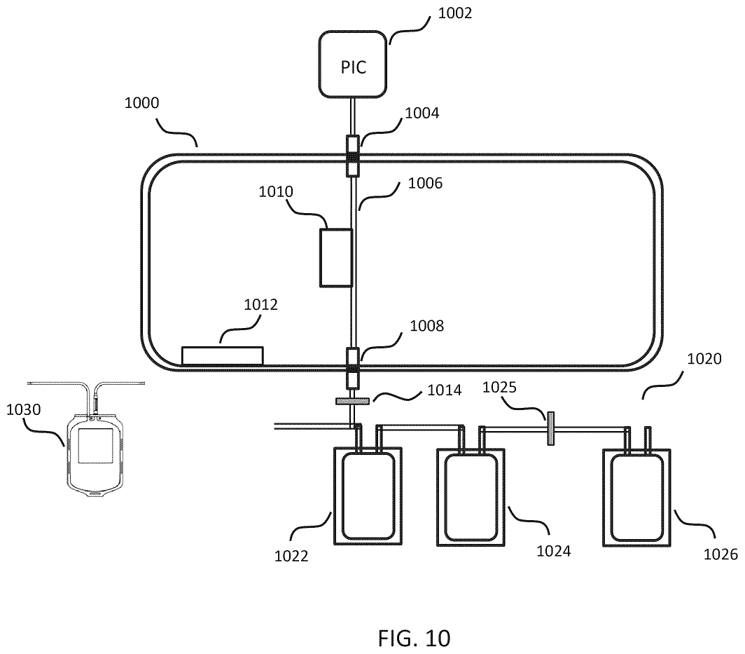

FIG. 10 depicts an exemplary system (e.g., dosing device system) for preparing a pathogen-inactivated blood composition, in accordance with some embodiments. The depicted system includes dosing device 1000 (e.g., a device comprising one or more components configured to control flow of one or more fluids of the present disclosure), one or more device-associated consumable components (e.g., reusable consumables) such as for example fluid path 1006, connectors 1004 and/or 1008 and optional pump 1010, and processing set 1020 (e.g., a processing set of the present disclosure). In some embodiments, connectors 1004 and/or 1008 may be a portion of a connector, with the other portion of the connector a part of processing set 1020 and/or container 1002 (e.g., each a portion that is configured to join with the other portion). Device 1000 comprises pump 1010 and/or a controller and/or actuator of pump 1010, and optional scale 1012. In this example, PIC container 1002 is coupled (e.g., reversibly, sterile coupled) to fluid path 1006 via connector 1004, which connects to fluid path 1006 and connector 1008 downstream. Pump 1010 may be connected to or otherwise associated with (e.g., loaded, mounted, attached, coupled) fluid path 1006 and controls flow of fluid (e.g., comprising a PIC of the present disclosure) through fluid path 1006, e.g., from PIC container 1002 through connector 1004 into fluid path 1006, and subsequently through connector 1008 into processing set 1020 (specifically, into mixing and/or illumination container 1022) optionally through filter 1014. Processing set 1020 may be connected to connector 1008 via optional filter 1014. Alternatively, or in addition, processing set 1020 may comprise a portion of fluid path (e.g., tubing) which comprises optional filter 1014, and the fluid path may be connected to connector 1008. This allows for transferring a determined amount of PIC from the PIC container into one or more containers of processing set 1020 in a sterile manner. In this example, processing set 1020 comprises mixing and/or illumination container 1022, CAD container 1024, optional filter 1025, and storage container 1026. Connectors 1004 and 1008 can be the same type of connector of the present disclosure, or different types of connectors of the present disclosure. In some embodiments, device 1000 comprises a housing. Optional scale 1012 can be within or outside of the housing. Scale 1012 can be used to weigh a blood composition of the present disclosure. In some embodiments, the system and/or device of FIG. 10 may comprise control circuitry and a computer system (not depicted) substantially similar to the system and/or device described for FIG. 8.

Blood composition container 1030 comprises a blood composition of the present disclosure. Blood composition container 1030 can be coupled (e.g., sterile coupled) to processing set 1020, allowing for the blood composition to be transferred into processing set 1020 (e.g., before, during, or after coupling processing set 1020 to fluid path 1006 and/or transfer of the PIC into processing set 1020). In some embodiments, the blood composition is a platelet and/or plasma composition. In some embodiments, the PIC is a photoactive pathogen inactivating compound of the present disclosure, e.g., a psoralen such as amotosalen. Processing set 1020 includes mixing and/or illumination container 1022, CAD container 1024, and storage container 1026. After transfer of the blood composition from container 1030 into container 1022, and the PIC from container 1002 into container 1022 via device 1000, the PIC can be admixed with the blood composition and/or subjected to illumination (e.g., for photochemical pathogen inactivation) in container 1022. Following illumination, the pathogen-inactivated blood composition can be transferred into CAD container 1024, e.g., for reducing the concentration of pathogen inactivating compound and by-products thereof by contacting the pathogen-inactivated blood composition with a CAD of the present disclosure. Following the CAD container, the pathogen-inactivated blood composition can be transferred into storage container 1026 (optionally through filter 1025), e.g., for storage. In some embodiments, the blood composition comprises RBCs (e.g., an RBC or whole blood composition). In some embodiments, the PIC comprises a nucleic acid binding ligand of the present disclosure that is an intercalator, e.g., an acridine such as β-alanine, N-(acridin-9-yl), 2-[bis(2-chloroethyl)amino]ethyl ester.

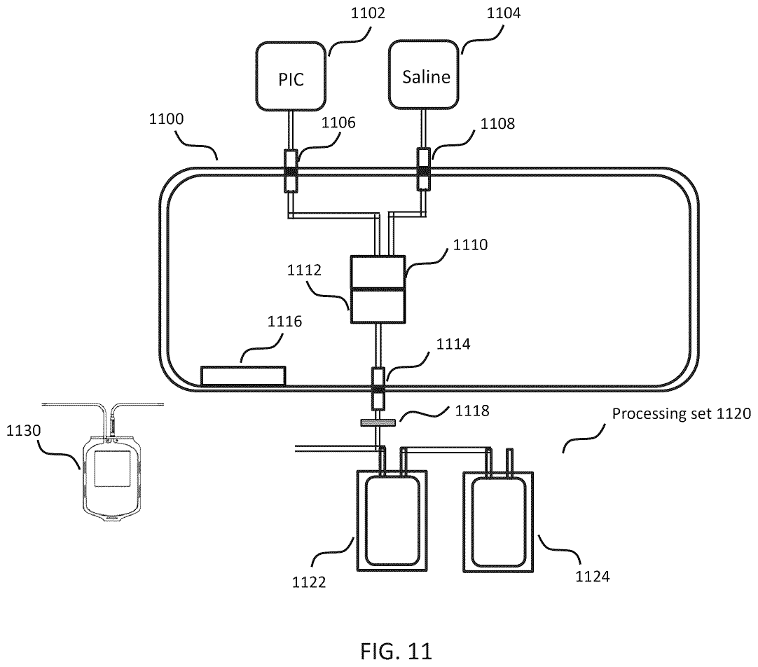

FIG. 11 depicts an exemplary system (e.g., dosing device system) for preparing a pathogen-inactivated blood composition, in accordance with some embodiments. The depicted system includes dosing device 1100 (e.g., a device comprising one or more components configured to control flow of one or more fluids of the present disclosure), one or more device-associated consumable components (e.g., reusable consumables) such as for example fluid path(s), connectors 1106, 1108 and/or 1114 and optional manifold (e.g., valve/manifold) 1110 and optional pump 1112, and processing set 1120 (e.g., a processing set of the present disclosure). In some embodiments, connectors 1106, 1108 and/or 1118 may be a portion of a connector, with the other portion of the connector a part of processing set 1120 and/or containers 1102 and/or 1104 (e.g., each a portion that is configured to join with the other portion). Device 1100 comprises manifold (e.g., valve/manifold) 1110 and/or a controller and/or actuator of manifold 1110; pump 1112 and/or a controller and/or actuator of pump 1110; and optional scale 1116. In this example, PIC container 1102 is coupled (e.g., reversibly, sterile coupled) to the fluid path (e.g., a first fluid path) via connector 1106, which connects to the fluid path, manifold 1110 and/or pump 1112 (connected or otherwise associated with (e.g., loaded, mounted, attached, coupled)), and connector 1114 downstream. Diluent container 1104 is coupled (e.g., reversibly, sterile coupled) to the fluid path via connector 1108, which connects to the fluid path, manifold 1110 and/or pump 1112 (connected or otherwise associated with (e.g., loaded, mounted, attached, coupled)), and connector 1114 downstream. This allows for delivery of a diluent (e.g., mixing with PIC) of the present disclosure (e.g., saline solution). In some embodiments, connectors 1106 and 1108 are connected to separate fluid paths to manifold 1110 and/or pump 1112. Pump 1112 and manifold 1110 control flow of fluid (e.g., comprising a PIC and/or diluent of the present disclosure) through the fluid path(s), e.g., from PIC container 1102 through connector 1106 into the fluid path and from diluent container 1104 through connector 1108 into the fluid path (optionally a second, separate fluid path), and subsequently through connector 1114 into processing set 1120 (specifically, into mixing and/or illumination container 1122) optionally through filter 1118. In some embodiments, valve/manifold 1110 can independently control fluid from container 1102 (e.g., comprising a PIC) and fluid from container 1104 (e.g., comprising a diluent) and/or can keep the PIC and diluent separate (e.g., prior to mixing in container 1122). In some embodiments, an additional fluid path can be coupled to manifold 1110 for transfer of fluids to and from a container configured for mixing the PIC and diluent (e.g., dilution container). In some embodiments, an additional fluid path can be coupled to manifold 1110, e.g., for transfer of fluids into a waste container or line. Processing set 1120 may be connected to connector 1114 via optional filter 1118. Alternatively, or in addition, processing set 1120 may comprise a portion of fluid path (e.g., tubing) which comprises optional filter 1118, and the fluid path may be connected to connector 1114. This allows for transferring a determined amount of PIC from the PIC container, as well as diluent from the diluent container, into one or more containers of processing set 1120 in a sterile manner. In this example, processing set 1120 comprises mixing and/or illumination container 1122 and storage container 1124. Connectors 1106, 1108, and 1114 can be the same type of connector of the present disclosure, or different types of connectors of the present disclosure. In some embodiments, device 1100 comprises a housing. Optional scale 1116 can be within or outside of the housing. Scale 1116 can be used to weigh a blood composition of the present disclosure. In some embodiments, the system and/or device of FIG. 11 may comprise control circuitry and a computer system (not depicted) substantially similar to the system and/or device described for FIG. 8.

Blood composition container 1130 comprises a blood composition of the present disclosure. Blood composition container 1130 can be coupled (e.g., sterile coupled) to processing set 1120, allowing for the blood composition to be transferred into processing set 1120 (e.g., before, during, or after coupling processing set 1120 to fluid path 1106 and/or transfer of the PIC and/or diluent into processing set 1120). In some embodiments, the blood composition is a platelet and/or plasma composition. In some embodiments, the PIC is a photoactive pathogen inactivating compound of the present disclosure, e.g., a psoralen such as amotosalen. Processing set 1120 includes mixing and/or illumination container 1122 and storage container 1124. After transfer of the blood composition from container 1130 into container 1122, the PIC from container 1102 into container 1122, and the diluent from container 1104 into container 1122 via device 1100, the PIC and diluent can be admixed with the blood composition and/or subjected to illumination (e.g., for photochemical pathogen inactivation) in container 1122. Following illumination, the pathogen-inactivated blood composition can be transferred into storage container 1124, e.g., for storage. In some embodiments, the blood composition comprises RBCs (e.g., an RBC or whole blood composition). In some embodiments, the PIC comprises a nucleic acid binding ligand of the present disclosure that is an intercalator, e.g., an acridine such as β-alanine, N-(acridin-9-yl), 2-[bis(2-chloroethyl)amino]ethyl ester.

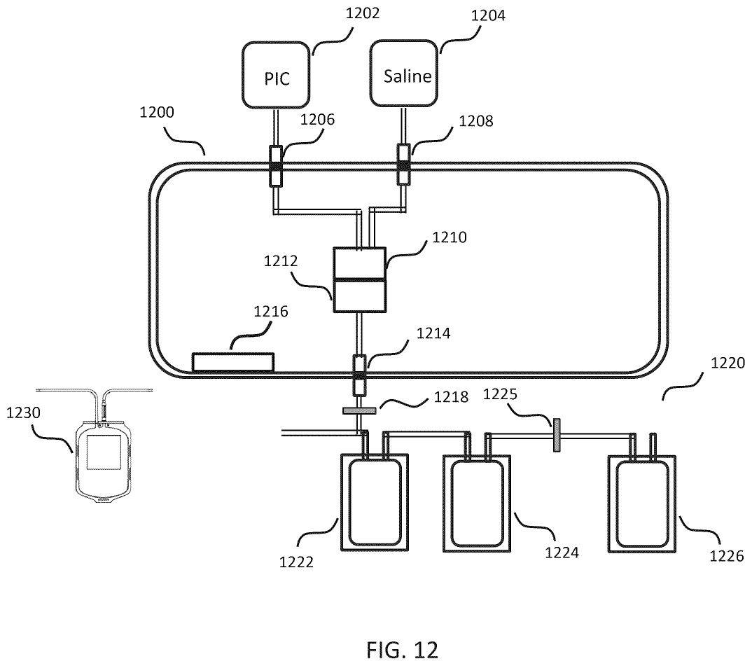

FIG. 12 depicts an exemplary system (e.g., dosing device system) for preparing a pathogen-inactivated blood composition, in accordance with some embodiments. The depicted system includes dosing device 1200 (e.g., a device comprising one or more components configured to control flow of one or more fluids of the present disclosure), one or more device-associated consumable components (e.g., reusable consumables) such as for example fluid path(s), connectors 1206, 1208 and/or 1214 and optional manifold (e.g., manifold/valve) 1210 and optional pump 1212, and processing set 1220 (e.g., a processing set of the present disclosure). In some embodiments, connectors 1206, 1208 and/or 1214 may be a portion of a connector, with the other portion of the connector a part of processing set 1220 and/or containers 1202 and/or 1204 (e.g., each a portion that is configured to join with the other portion). Device 1200 comprises manifold (e.g., valve/manifold) 1210 and/or a controller or actuator of manifold 1210; pump 1212 and/or a controller or actuator of pump 1212; and optional scale 1216. In this example, PIC container 1202 is coupled (e.g., reversibly, sterile coupled) to the fluid path (e.g., a first fluid path) via connector 1206, which connects to the fluid path, manifold 1210 and/or pump 1212 (connected or otherwise associated with (e.g., loaded, mounted, attached, coupled)) and connector 1214 downstream. Diluent container 1204 is coupled (e.g., reversibly, sterile coupled) to to the fluid path via connector 1208, which connects to the fluid path, manifold 1210 and/or pump 1212 (connected or otherwise associated with (e.g., loaded, mounted, attached, coupled)), and connector 1214 downstream. This allows for delivery of a diluent (e.g., mixing with PIC) of the present disclosure (e.g., saline solution). In some embodiments, connectors 1206 and 1208 are connected to separate fluid paths into valve/manifold 1210 and/or pump 1212. Pump 1212 and manifold 1210 control flow of fluid (e.g., comprising a PIC and/or diluent of the present disclosure) through the fluid path(s), e.g., from PIC container 1202 through connector 1206 into the fluid path and from diluent container 1204 through connector 1208 into the fluid path, and subsequently through connector 1214 into processing set 1220 (specifically, into mixing and/or illumination container 1222) optionally through filter 1218. In some embodiments, valve/manifold 1210 can independently control fluid from container 1202 (e.g., comprising a PIC) and fluid from container 1204 (e.g., comprising a diluent) and/or can keep the PIC and diluent separate (e.g., prior to mixing in container 1222). In some embodiments, an additional fluid path can be coupled to manifold 1110 for transfer of fluids to and from a container configured for mixing the PIC and diluent (e.g., dilution container). In some embodiments, an additional fluid path can be coupled to valve/manifold 1210, e.g., for transfer of fluids into a waste container or line. Processing set 1220 may be connected to connector 1214 via optional filter 1218. Alternatively, or in addition, processing set 1220 may comprise a portion of fluid path (e.g., tubing) which comprises optional filter 1218, and the fluid path may be connected to connector 1214. This allows for transferring a determined amount of PIC from the PIC container, as well as diluent from the diluent container, into one or more containers of processing set 1220 in a sterile manner. In this example, processing set 1220 comprises mixing and/or illumination container 1222, CAD container 1224, optional filter 1225, and storage container 1226. Connectors 1206, 1208, and 1214 can be the same type of connector of the present disclosure, or different types of connectors of the present disclosure. In some embodiments, device 1200 comprises a housing. Optional scale 1216 can be within or outside of the housing. Scale 1216 can be used to weigh a blood composition of the present disclosure. In some embodiments, the system and/or device of FIG. 12 may comprise control circuitry and a computer system (not depicted) substantially similar to the system and/or device described for FIG. 8.

Blood composition container 1230 comprises a blood composition of the present disclosure. Blood composition container 1230 can be coupled (e.g., sterile coupled) to processing set 1220, allowing for the blood composition to be transferred into processing set 1220 (e.g., before, during, or after coupling processing set 1220 to fluid path 1206 and/or transfer of the PIC and/or diluent into processing set 1220). In some embodiments, the blood composition is a platelet and/or plasma composition. In some embodiments, the PIC is a photoactive pathogen inactivating compound of the present disclosure, e.g., a psoralen such as amotosalen. Processing set 1220 includes mixing and/or illumination container 1222, CAD container 1224, and storage container 1226. After transfer of the blood composition from container 1230 into container 1222, the PIC from container 1202 into container 1222 via device 1200, and the diluent from container 1204 into container 1222 via device 1200, the PIC and diluent can be admixed with the blood composition and/or subjected to illumination (e.g., for photochemical pathogen inactivation) in container 1222. Following illumination, the pathogen-inactivated blood composition can be transferred into CAD container 1224, e.g., for reducing the concentration of pathogen inactivating compound and by-products thereof by contacting the pathogen-inactivated blood composition with a CAD of the present disclosure. Following the CAD container, the pathogen-inactivated blood composition can be transferred into storage container 1226 (optionally through filter 1225), e.g., for storage. In some embodiments, the blood composition comprises RBCs (e.g., an RBC or whole blood composition). In some embodiments, the PIC comprises a nucleic acid binding ligand of the present disclosure that is an intercalator, e.g., an acridine such as β-alanine, N-(acridin-9-yl), 2-[bis(2-chloroethyl)amino]ethyl ester.

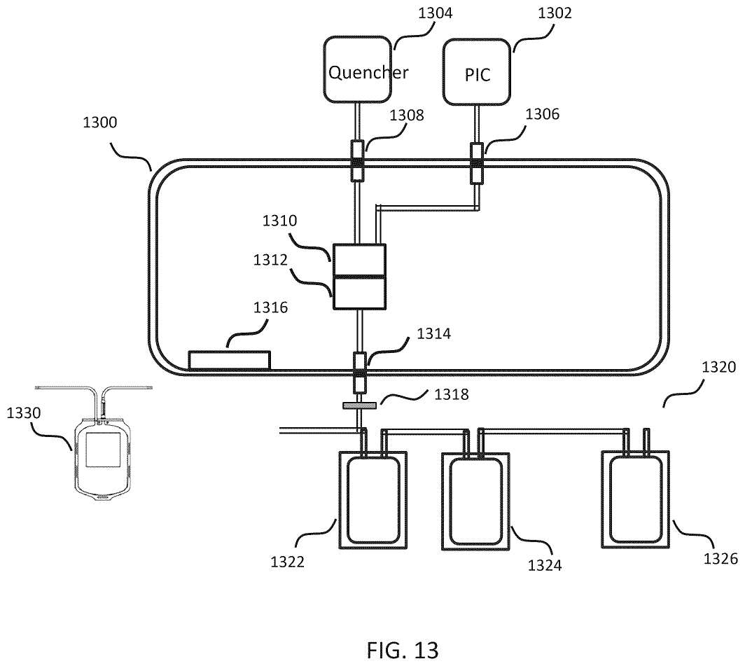

FIG. 13 depicts an exemplary system (e.g., dosing device system) for preparing a pathogen-inactivated blood composition, in accordance with some embodiments. The depicted system includes dosing device 1300 (e.g., a device comprising one or more components configured to control flow of one or more fluids of the present disclosure), one or more device-associated consumable components (e.g., reusable consumables) such as for example fluid path(s), connectors 1306, 1308 and/or 1314 and optional manifold (e.g., manifold/valve) 1310 and optional pump 1312, and processing set 1320 (e.g., a processing set of the present disclosure). In some embodiments, connectors 1306, 1308 and/or 1314 may be a portion of a connector, with the other portion of the connector a part of processing set 1320 and/or containers 1302 and/or 1304 (e.g., each a portion that is configured to join with the other portion). Device 1300 comprises manifold (e.g., valve/manifold) 1310 and/or a controller and/or actuator of manifold 1310; pump 1312 and/or a controller and/or actuator of pump 1312; and optional scale 1316. In this example, PIC container 1302 is coupled (e.g., reversibly, sterile coupled) to a fluid path (e.g., a first fluid path) via connector 1306, which connects to the fluid path, manifold 1310 and/or pump 1312 (connected or otherwise associated with (e.g., loaded, mounted, attached, coupled)), and connector 1314 downstream. Quencher container 1304 is coupled (e.g., reversibly, sterile coupled) to the fluid path via connector 1308, which connects to a fluid path, manifold 1310 and/or pump 1312 (connected or otherwise associated with (e.g., loaded, mounted, attached, coupled)), and connector 1314 downstream. This allows for delivery of a quencher of the present disclosure (e.g., glutathione or a pharmaceutically acceptable salt thereof). In some embodiments, the PIC and quencher are delivered via separate fluid paths to manifold 1310 and/or pump 1312. In some embodiments, connectors 1306 and 1308 are connected to separate fluid paths to manifold 1310 and/or pump 1312. Pump 1312 and manifold 1310 control flow of fluid (e.g., comprising a PIC and/or quencher of the present disclosure) through the fluid path, e.g., from PIC container 1302 through connector 1306 into a fluid path and from quencher container 1304 through connector 1308 into a fluid path, and subsequently through connector 1314 into processing set 1320 (specifically, into mixing container 1322) optionally through filter 1318. In some embodiments, manifold 1310 can independently control fluid from container 1302 (e.g., comprising a PIC) and fluid from container 1304 (e.g., comprising a quencher) and/or can keep the PIC and quencher separate (e.g., prior to mixing in container 1322). In some embodiments, an additional fluid path can be coupled to valve/manifold 1310, e.g., for transfer of fluids into a waste container or line. Processing set 1320 may be connected to connector 1314 via optional filter 1318. Alternatively, or in addition, processing set 1320 may comprise a portion of fluid path (e.g., tubing) which comprises optional filter 1318, and the fluid path may be connected to connector 1314. This allows for transferring a determined amount of PIC from the PIC container, as well as quencher from the quencher container, into one or more containers of processing set 1320 in a sterile manner. In this example, processing set 1320 comprises mixing container 1322, incubation container 1324, and storage container (e.g., container with additive solution) 1326. In some embodiments, mixing container 1322 contains a processing solution (e.g., prior to addition of the blood composition, PIC, and/or quencher). Connectors 1306, 1308, and 1314 can be the same type of connector of the present disclosure, or different types of connectors of the present disclosure. In some embodiments, device 1300 comprises a housing. Optional scale 1316 can be within or outside of the housing. Scale 1316 can be used to weigh a blood composition of the present disclosure. In some embodiments, the system and/or device of FIG. 13 may comprise control circuitry and a computer system (not depicted) substantially similar to the system and/or device described for FIG. 8.

Blood composition container 1330 comprises a blood composition of the present disclosure. Blood composition container 1330 can be coupled (e.g., sterile coupled) to processing set 1320, allowing for the blood composition to be transferred into processing set 1320 (e.g., before, during, or after coupling processing set 1320 to fluid path 1306 and/or transfer of the PIC and/or quencher into processing set 1320). Processing set 1320 includes mixing container 1322, incubation container 1324, and additive solution container 1326. After transfer (e.g., by sterile transfer) of the blood composition into container 1322 and mixing with PIC and quencher, the mixture can be transferred (e.g., by sterile transfer) to incubation container 1324 for incubation, followed by transfer (e.g., by sterile transfer) into additive solution in container 1326. In some embodiments, the blood composition comprises RBCs (e.g., an RBC or whole blood composition). In some embodiments, the PIC comprises a nucleic acid binding ligand of the present disclosure that is an intercalator, e.g., an acridine such as β-alanine, N-(acridin-9-yl), 2-[bis(2-chloroethyl)amino]ethyl ester. In some embodiments, the quencher comprises cysteine or a derivative of cysteine, e.g., a peptide of 3-6 amino acids, wherein at least one of the amino acids is cysteine, N-acetyl cysteine, or S-acetyl cysteine. In some embodiments, the quencher is glutathione or a pharmaceutically acceptable salt thereof, e.g., glutathione monosodium salt.

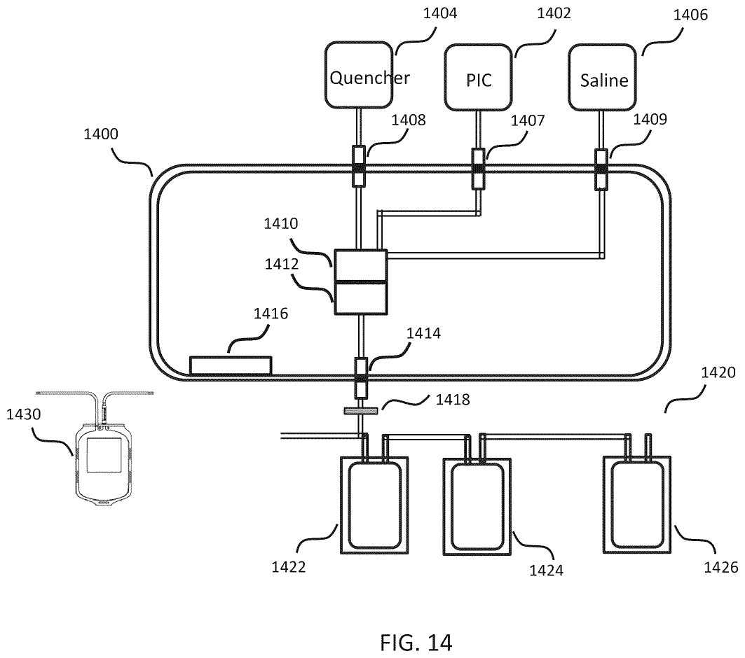

FIG. 14 depicts an exemplary system (e.g., dosing device system) for preparing a pathogen-inactivated blood composition, in accordance with some embodiments. The depicted system includes dosing device 1400 (e.g., a device comprising one or more components configured to control flow of one or more fluids of the present disclosure), one or more device-associated consumable components (e.g., reusable consumables) such as for example fluid path(s), connectors 1407, 1408, 1409, and/or 1414 and optional manifold (e.g., manifold/valve) 1410 and optional pump 1412, and processing set 1420 (e.g., a processing set of the present disclosure). In some embodiments, connectors 1407, 1408, 1409, and/or 1414 may be a portion of a connector, with the other portion of the connector a part of processing set 1420 and/or containers 1402, 1404, and/or 1406 (e.g., each a portion that is configured to join with the other portion). Device 1400 comprises manifold (e.g., valve/manifold) 1410 and/or a controller and/or actuator of manifold 1410; pump 1412 and/or a controller and/or actuator of pump 1412; and optional scale 1416. In this example, PIC container 1402 is coupled (e.g., reversibly, sterile coupled) to a fluid path (e.g., a first fluid path) via connector 1407, which connects to a fluid path, manifold 1410 and/or pump 1412 (connected to or otherwise associated with (e.g., loaded, mounted, attached, coupled)), and connector 1414 downstream. Quencher container 1404 is coupled (e.g., reversibly, sterile coupled) to a fluid path via connector 1408, which connects to a fluid path, manifold 1410 and/or pump 1412 (connected to or otherwise associated with (e.g., loaded, mounted, attached, coupled)), and connector 1414 downstream. This allows for delivery of a quencher of the present disclosure (e.g., glutathione or a pharmaceutically acceptable salt thereof). Diluent container 1406 is coupled (e.g., reversibly, sterile coupled) to a fluid path via connector 1409, which connects to a fluid path, manifold 1410 and/or pump 1412 (connected to or otherwise associated with (e.g., loaded, mounted, attached, coupled)), and connector 1414 downstream. This allows for delivery of a diluent (e.g., mixing with PIC) of the present disclosure (e.g., saline solution). In some embodiments, an additional fluid path can be coupled to manifold 1410 for transfer of fluids to and from a container configured for mixing the PIC and diluent (e.g., dilution container). In some embodiments, the PIC, quencher, and diluent are delivered via separate fluid paths to manifold 1410 and/or pump 1412. In some embodiments, connectors 1407, 1408, and 1409 are connected to separate fluid paths to manifold 1410 and/or pump 1412. Pump 1412 and manifold 1410 control flow of fluid (e.g., comprising a PIC, quencher, and/or diluent of the present disclosure) through the fluid path(s), e.g., from PIC container 1402 through connector 1407 into a fluid path, from quencher container 1404 through connector 1408 into a fluid path, and from diluent container 1406 through connector 1409 into a fluid path, and subsequently through connector 1414 into processing set 1420 (specifically, into mixing container 1422) optionally through filter 1418. In some embodiments, manifold 1410 can independently control fluid from container 1402 (e.g., comprising a PIC), fluid from container 1404 (e.g., comprising a quencher), and fluid from container 1406 (e.g., comprising a diluent) and/or can keep the PIC quencher, and/or diluent separate (e.g., prior to mixing in container 1422, prior to mixing in a dilution container). In some embodiments, an additional fluid path can be coupled to manifold 1410 for transfer of fluids to and from a container configured for mixing the PIC and diluent (e.g., dilution container). In some embodiments, an additional fluid path can be coupled to manifold 1410, e.g., for transfer of fluids into a waste container or line. Processing set 1420 may be connected to connector 1414 via optional filter 1418. Alternatively, or in addition, processing set 1420 may comprise a portion of fluid path (e.g., tubing) which comprises optional filter 1418, and the fluid path may be connected to connector 1414. This allows for transferring a determined amount of PIC from the PIC container, quencher from the quencher container, and diluent from the diluent container into one or more containers of processing set 1420 in a sterile manner. In this example, processing set 1420 comprises mixing container 1422, incubation container 1424, and storage container (e.g., container with additive solution) 1426. In some embodiments, mixing container 1422 contains a processing solution (e.g., prior to addition of the blood composition, PIC, quencher, and/or diluent). Connectors 1407, 1408, 1409, and 1414 can be the same type of connector of the present disclosure, or different types of connectors of the present disclosure. In some embodiments, device 1400 comprises a housing. Optional scale 1416 can be within or outside of the housing. Scale 1416 can be used to weigh a blood composition of the present disclosure. In some embodiments, the system and/or device of FIG. 14 may comprise control circuitry and a computer system (not depicted) substantially similar to the system and/or device described for FIG. 8.

Blood composition container 1430 comprises a blood composition of the present disclosure. Blood composition container 1430 can be coupled (e.g., sterile coupled) to processing set 1420, allowing for the blood composition to be transferred into processing set 1420 (e.g., before, during, or after coupling processing set 1420 to fluid path 1406 and/or transfer of the PIC, quencher, and/or diluent into processing set 1420). Processing set 1420 includes mixing container 1422, incubation container 1424, and additive solution container 1426. After transfer (e.g., by sterile transfer) of the blood composition into container 1422 and mixing with PIC and quencher, the mixture can be transferred (e.g., by sterile transfer) to incubation container 1424 for incubation, followed by transfer (e.g., by sterile transfer) into additive solution in container 1426. In some embodiments, the blood composition comprises RBCs (e.g., an RBC or whole blood composition). In some embodiments, the PIC comprises a nucleic acid binding ligand of the present disclosure that is an intercalator, e.g., an acridine such as β-alanine, N-(acridin-9-yl), 2-[bis(2-chloroethyl)amino]ethyl ester. In some embodiments, the quencher comprises cysteine or a derivative of cysteine, e.g., a peptide of 3-6 amino acids, wherein at least one of the amino acids is cysteine, N-acetyl cysteine, or S-acetyl cysteine. In some embodiments, the quencher is glutathione or a pharmaceutically acceptable salt thereof, e.g., glutathione monosodium salt.

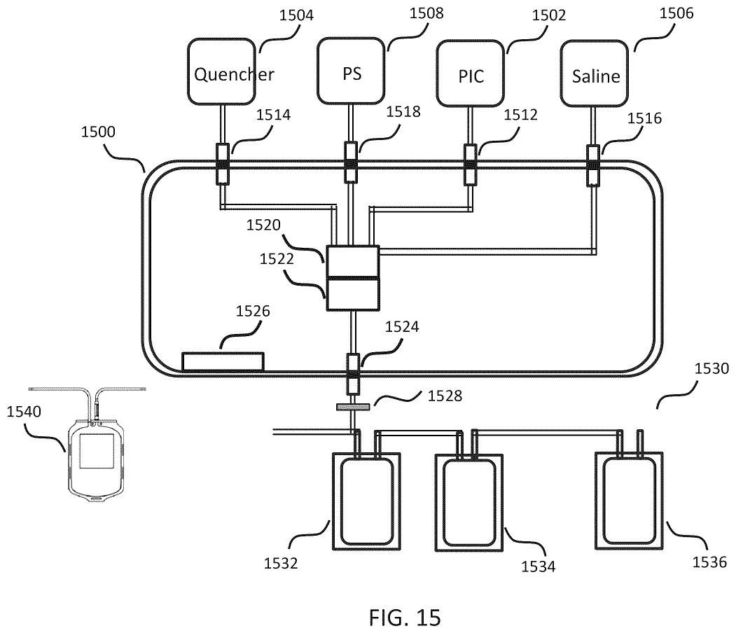

FIG. 15 depicts an exemplary system (e.g., dosing device system) for preparing a pathogen-inactivated blood composition, in accordance with some embodiments. The depicted system includes dosing device 1500 (e.g., a device comprising one or more components configured to control flow of one or more fluids of the present disclosure), one or more device-associated consumable components (e.g., reusable consumables) such as for example fluid path(s), connectors 1512, 1514, 1516, 1518 and/or 1524, and optional manifold (e.g., manifold/valve) 1520 and optional pump 1522, and processing set 1530 (e.g., a processing set of the present disclosure). In some embodiments, connectors 1512, 1514, 1516, 1518 and/or 1524 may be a portion of a connector, with the other portion of the connector a part of processing set 1530 and/or containers 1502, 1504, 1506, and/or 1508 (e.g., each a portion that is configured to join with the other portion). Device 1500 comprises manifold (e.g., valve/manifold) 1520 and/or a controller and/or actuator of manifold 1520; pump 1522 and/or a controller and/or actuator of pump 1522; and optional scale 1526. In this example, PIC container 1502 is coupled (e.g., reversibly, sterile coupled) to a fluid path (e.g., first fluid path) via connector 1512, which connects to a fluid path, manifold 1520 and/or pump 1522 (connected to or otherwise associated with (e.g., loaded, mounted, attached, coupled)), and connector 1524 downstream. Quencher container 1504 is coupled (e.g., sterile coupled) to a fluid path via connector 1514, which connects to a fluid path, manifold 1520 and/or pump 1522 (connected to or otherwise associated with (e.g., loaded, mounted, attached, coupled)), and connector 1524 downstream. This allows for delivery of a quencher of the present disclosure (e.g., glutathione or a pharmaceutically acceptable salt thereof). Diluent container 1506 is coupled (e.g., sterile coupled) to a fluid path via connector 1516, which connects to a fluid path, manifold 1520 and/or pump 1522 (connected to or otherwise associated with (e.g., loaded, mounted, attached, coupled)), and connector 1524 downstream. This allows for delivery of a diluent of the present disclosure (e.g., saline solution). In some embodiments, an additional fluid path can be coupled to manifold 1410 for transfer of fluids to and from a container configured for mixing the PIC and diluent (e.g., dilution container). Processing solution container 1508 is coupled (e.g., sterile coupled) to a fluid path) via connector 1518, which connects to a fluid path, manifold 1520 and/or pump 1522 (connected to or otherwise associated with (e.g., loaded, mounted, attached, coupled)), and connector 1524 downstream. This allows for delivery of a processing solution of the present disclosure. In some embodiments, the PIC, quencher, diluent, and processing solution are delivered via separate fluid paths into manifold 1520 and/or pump 1522. In some embodiments, connectors 1512, 1514, 1516, and 1518 are connected to separate fluid paths into manifold 1520 and/or pump 1522. Pump 1522 and manifold 1520 control flow of fluid (e.g., comprising a PIC, quencher, diluent, and/or processing solution of the present disclosure) through the fluid path, e.g., from PIC container 1502 through connector 1512 into a fluid path, from quencher container 1504 through connector 1514 into a fluid path, from diluent container 1506 through connector 1516 into a fluid path, and from processing solution container 1508 through connector 1518 into a fluid path, and subsequently through connector 1524 into processing set 1530 (specifically, into mixing container 1532) optionally through filter 1528. In some embodiments, valve/manifold 1520 can independently control fluid from container 1502 (e.g., comprising a PIC), fluid from container 1504 (e.g., comprising a quencher), fluid from container 1506 (e.g., comprising a diluent), and fluid from container 1508 (e.g., comprising a processing solution) and/or can keep the PIC, quencher, diluent, and/or processing solution separate (e.g., prior to mixing in container 1532; prior to mixing in a dilution container). In some embodiments, an additional fluid path can be coupled to manifold 1520 for transfer of fluids to and from a container configured for mixing the PIC and diluent (e.g., dilution container). In some embodiments, an additional fluid path can be coupled to manifold 1520, e.g., for transfer of fluids into a waste container or line. Processing set 1530 may be connected to connector 1524 via optional filter 1528. Alternatively, or in addition, processing set 1530 may comprise a portion of fluid path (e.g., tubing) which comprises optional filter 1528, and the fluid path may be connected to connector 1524. This allows for transferring a determined amount of PIC from the PIC container, quencher from the quencher container, diluent from the diluent container, and processing solution from the processing solution container into one or more containers of processing set 1530 in a sterile manner. In this example, processing set 1530 comprises mixing container 1532, incubation container 1534, and storage container (e.g., container with additive solution) 1536. Connectors 1512, 1514, 1516, 1518, and 1524 can be the same type of connector of the present disclosure, or different types of connectors. In some embodiments, device 1500 comprises a housing. Optional scale 1526 can be within or outside of the housing. Scale 1526 can be used to weigh a blood composition of the present disclosure. In some embodiments, the system and/or device of FIG. 15 may comprise control circuitry and a computer system (not depicted) substantially similar to the system and/or device described for FIG. 8.

Blood composition container 1540 comprises a blood composition of the present disclosure. Blood composition container 1540 can be coupled (e.g., sterile coupled) to processing set 1530, allowing for the blood composition to be transferred into processing set 1530 (e.g., before, during, or after coupling processing set 1520 to fluid path 1506 and/or transfer of the PIC, quencher, diluent, and/or processing solution into processing set 1530). Processing set 1530 includes mixing container 1532, incubation container 1534, and additive solution container 1536. After transfer (e.g., by sterile transfer) of the blood composition into container 1322 and mixing with PIC and quencher, the mixture can be transferred (e.g., by sterile transfer) to incubation container 1324 for incubation, followed by transfer (e.g., by sterile transfer) into additive solution in container 1326. In some embodiments, the blood composition comprises RBCs (e.g., an RBC or whole blood composition). In some embodiments, the PIC comprises a nucleic acid binding ligand of the present disclosure that is an intercalator, e.g., an acridine such as β-alanine, N-(acridin-9-yl), 2-[bis(2-chloroethyl)amino]ethyl ester. In some embodiments, the quencher comprises cysteine or a derivative of cysteine, e.g., a peptide of 3-6 amino acids, wherein at least one of the amino acids is cysteine, N-acetyl cysteine, or S-acetyl cysteine. In some embodiments, the quencher is glutathione or a pharmaceutically acceptable salt thereof, e.g., glutathione monosodium salt.

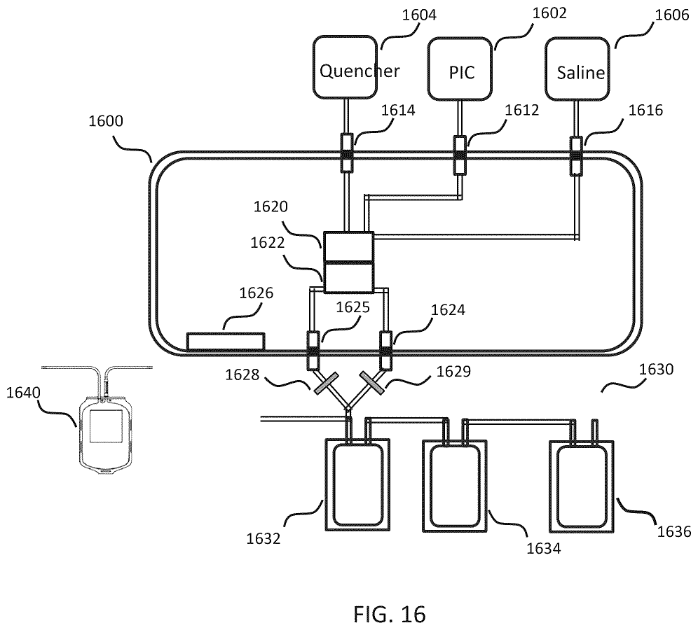

FIG. 16 depicts an exemplary system (e.g., dosing device system) for preparing a pathogen-inactivated blood composition, in accordance with some embodiments. The depicted system includes dosing device 1600 (e.g., a device comprising one or more components configured to control flow of one or more fluids of the present disclosure), one or more device-associated consumable components (e.g., reusable consumables) such as for example fluid path(s), connectors 1612, 1614, 1616, 1624 and/or 1625, and optional manifold (e.g., manifold/valve) 1610 and optional pump 1612, and processing set 1630 (e.g., a processing set of the present disclosure). In some embodiments, connectors 1612, 1614, 1616, 1624 and/or 1625 may be a portion of a connector, with the other portion of the connector a part of processing set 1630 and/or containers 1602, 1604, and/or 1606 (e.g., each a portion that is configured to join with the other portion). Device 1600 comprises a manifold (e.g., valve/manifold) 1620 and/or controller and/or actuator of manifold 1620; pump 1622 and/or controller and/or actuator of pump 1622; and optional scale 1626. In this example, PIC container 1602 is coupled (e.g., reversibly, sterile coupled) to a fluid path (e.g., first fluid path) via connector 1612, which connects to a fluid path, manifold 1620 and/or pump 1622 (connected to or otherwise associated with (e.g., loaded, mounted, attached, coupled)), and connector 1624 or 1625 downstream. Quencher container 1604 is coupled (e.g., reversibly, sterile coupled) to a fluid path via connector 1614, which connects to a fluid path, manifold 1620 and/or pump 1622 (connected to or otherwise associated with (e.g., loaded, mounted, attached, coupled)), and connector 1624 or 1625 downstream. This allows for delivery of a quencher of the present disclosure (e.g., glutathione or a pharmaceutically acceptable salt thereof). Diluent container 1606 is coupled (e.g., reversibly, sterile coupled) to a fluid path via connector 1616, which connects to a fluid path, manifold 1620 and/or pump 1622 (connected to or otherwise associated with (e.g., loaded, mounted, attached, coupled)), and connector 1624 or 1625 downstream. This allows for delivery of a diluent of the present disclosure (e.g., saline solution). In some embodiments, an additional fluid path can be coupled to manifold 1620 for transfer of fluids to and from a container configured for mixing the PIC and diluent (e.g., dilution container). In some embodiments, the PIC, quencher, and diluent are delivered via separate fluid paths into manifold 1620 and/or pump 1622. In some embodiments, connectors 1612, 1614, and 1616 are connected to separate fluid paths into manifold 1620 and/or pump 1622. Pump 1622 and manifold 1620 control flow of fluid (e.g., comprising a PIC, quencher, and/or diluent of the present disclosure) through the fluid paths, e.g., from PIC container 1602 through connector 1612 into a fluid path, from quencher container 1604 through connector 1614 into a fluid path, and from diluent container 1606 through connector 1616 into a fluid path, and subsequently through connector 1624 or 1625 into processing set 1630 (specifically, into mixing container 1632) optionally through filter 1628 or 1629. In some embodiments, manifold 1620 can independently control fluid from container 1602 (e.g., comprising a PIC), fluid from container 1604 (e.g., comprising a quencher), and fluid from container 1606 (e.g., comprising a diluent) and/or can keep the PIC, quencher, and/or diluent separate (e.g., prior to mixing in container 1632, prior to mixing in a dilution container). This configuration can allow for the PIC and quencher to be transferred in separate fluid paths, e.g., prior to mixing in container 1632. For example, PIC can be transferred from container 1602 and connector 1612, through valve/manifold 1620 and/or pump 1622, and through connector 1625 via a first fluid path into container 1632 (optionally through filter 1628), while quencher can be transferred from container 1604 and connector 1614, through valve/manifold 1620 and/or pump 1622, and through connector 1624 via a second fluid path into container 1632 (optionally through filter 1629). In some embodiments, an additional fluid path can be coupled to manifold 1620 for transfer of fluids to and from a container configured for mixing the PIC and diluent (e.g., dilution container). In some embodiments, an additional fluid path can be coupled to manifold 1620, e.g., for transfer of fluids into a waste container or line. Processing set 1630 may be connected to connector 1624 via optional filter 1629 and to connector 1625 via optional filter 1628. Alternatively, or in addition, processing set 1630 may comprise a portion of fluid path (e.g., tubing, with branch, with Y-junction) which comprises optional filters 1628 and 1629, and the fluid path may be connected to connectors 1624 and 1625. This allows for transferring a determined amount of PIC from the PIC container, quencher from the quencher container, and diluent from the diluent container into one or more containers of processing set 1630 in a sterile manner while keeping PIC and quencher separate. In this example, processing set 1630 comprises mixing container 1632, incubation container 1634, and storage container (e.g., container with additive solution) 1636. In some embodiments, mixing container 1632 contains a processing solution (e.g., prior to addition of the blood composition, PIC, quencher, and/or diluent). Connectors 1612, 1614, 1616, 1624, and 1625 can be the same type of connector of the present disclosure, or different types of connectors. In some embodiments, device 1600 comprises a housing. Optional scale 1626 can be within or outside of the housing. Scale 1626 can be used to weigh a blood composition of the present disclosure. In some embodiments, the system and/or device of FIG. 16 may comprise control circuitry and a computer system (not depicted) substantially similar to the system and/or device described for FIG. 8.

Blood composition container 1640 comprises a blood composition of the present disclosure. Blood composition container 1640 can be coupled (e.g., sterile coupled) to processing set 1630, allowing for the blood composition to be transferred into processing set 1630 (e.g., before, during, or after coupling processing set 1620 to fluid path 1606 and/or transfer of the PIC, quencher, and/or diluent into processing set 1630). Processing set 1630 includes mixing container 1632, incubation container 1634, and additive solution container 1636. After transfer (e.g., by sterile transfer) of the blood composition into container 1632 and mixing with PIC and quencher, the mixture can be transferred (e.g., by sterile transfer) to incubation container 1634 for incubation, followed by transfer (e.g., by sterile transfer) into additive solution in container 1636. In some embodiments, the blood composition comprises RBCs (e.g., an RBC or whole blood composition). In some embodiments, the PIC comprises a nucleic acid binding ligand of the present disclosure that is an intercalator, e.g., an acridine such as β-alanine, N-(acridin-9-yl), 2-[bis(2-chloroethyl)amino]ethyl ester. In some embodiments, the quencher comprises cysteine or a derivative of cysteine, e.g., a peptide of 3-6 amino acids, wherein at least one of the amino acids is cysteine, N-acetyl cysteine, or S-acetyl cysteine. In some embodiments, the quencher is glutathione or a pharmaceutically acceptable salt thereof, e.g., glutathione monosodium salt.

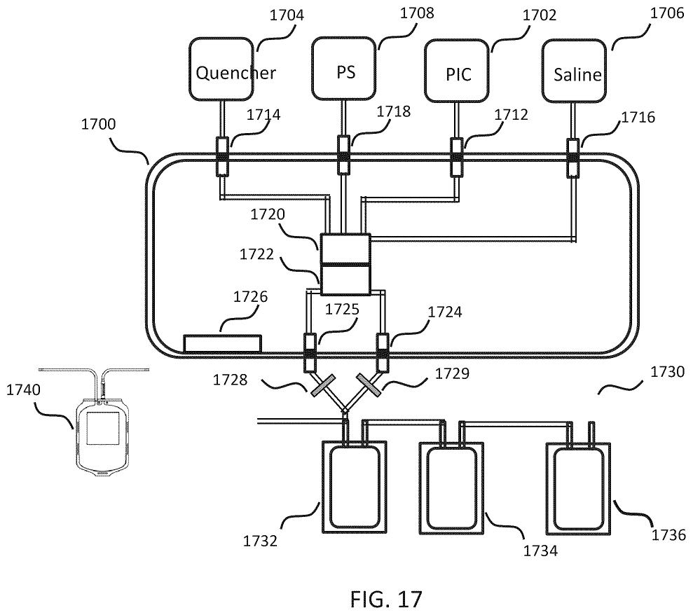

FIG. 17 depicts an exemplary system (e.g., dosing device system) for preparing a pathogen-inactivated blood composition, in accordance with some embodiments. The depicted system includes dosing device 1700 (e.g., a device comprising one or more components configured to control flow of one or more fluids of the present disclosure); one or more device-associated consumable components (e.g., reusable consumables) such as for example fluid path(s), connectors 1712, 1714, 1716, 1718, 1724 and/or 1725, and optional manifold (e.g., manifold/valve) 1720 and optional pump 1722; and processing set 1730 (e.g., a processing set of the present disclosure). In some embodiments, connectors 1712, 1714, 1716, 1718, 1724 and/or 1725 may be a portion of a connector, with the other portion of the connector a part of processing set 1730 and/or containers 1702, 1704, 1706, and/or 1708 (e.g., each a portion that is configured to join with the other portion). Device 1700 comprises a manifold (e.g., valve/manifold) 1720 and/or controller and/or actuator of manifold 1720; pump 1722 and/or controller and/or actuator of pump 1722; and optional scale 1726. In this example, PIC container 1702 is coupled (e.g., reversibly, sterile coupled) to a fluid path (e.g., first fluid path) via connector 1712, which connects to a fluid path, manifold 1720 and/or pump 1722 (connected to or otherwise associated with (e.g., loaded, mounted, attached, coupled)), and connector 1724 or 1725 downstream. Quencher container 1704 is coupled (e.g., reversibly, sterile coupled) to a fluid path) via connector 1714, which connects to a fluid path, manifold 1720 and/or pump 1722 (connected to or otherwise associated with (e.g., loaded, mounted, attached, coupled)), and connector 1724 or 1725 downstream. This allows for delivery of a quencher of the present disclosure (e.g., glutathione or a pharmaceutically acceptable salt thereof). Diluent container 1706 is coupled (e.g., reversibly, sterile coupled) to a fluid path via connector 1716, which connects to a fluid path, manifold 1720 and/or pump 1722 (connected to or otherwise associated with (e.g., loaded, mounted, attached, coupled)), and connector 1724 or 1725 downstream. This allows for delivery of a diluent of the present disclosure (e.g., saline solution).). In some embodiments, an additional fluid path can be coupled to manifold 1720 for transfer of fluids to and from a container configured for mixing the PIC and diluent (e.g., dilution container). Processing solution container 1708 is coupled (e.g., reversibly, sterile coupled) to a fluid path via connector 1718, which connects to a fluid path, manifold 1720 and/or pump 1722 (connected to or otherwise associated with (e.g., loaded, mounted, attached, coupled)), and connector 1724 or 1725 downstream. This allows for delivery of a processing solution of the present disclosure. In some embodiments, the PIC, quencher, diluent, and/or processing solution are delivered via separate fluid paths to manifold 1720 and/or pump 1722. Pump 1722 and manifold 1720 control flow of fluid (e.g., comprising a PIC, quencher, diluent, and/or processing solution of the present disclosure) through the fluid paths, e.g., from PIC container 1702 through connector 1712 into a fluid path, from quencher container 1704 through connector 1714 into a fluid path, from diluent container 1706 through connector 1716 into a fluid path, and from processing solution container 1708 through connector 1718 into a fluid path, and subsequently through connector 1724 or 1725 into processing set 1730 (specifically, into mixing container 1732) optionally through filter 1728 or 1729. In some embodiments, connectors 1712, 1714, 1716, and 1718 are connected to separate fluid paths to manifold 1720 and/or pump 1722. In some embodiments, manifold 1720 can independently control fluid from container 1702 (e.g., comprising a PIC), fluid from container 1704 (e.g., comprising a quencher), fluid from container 1706 (e.g., comprising a diluent), and fluid from container 1708 (e.g., comprising a processing solution) and/or can keep the PIC, quencher, diluent, and/or processing solution separate (e.g., prior to mixing in container 1732). This configuration can allow for the PIC and quencher to be transferred in separate fluid paths, e.g., prior to mixing in container 1732. For example, PIC can be transferred from container 1702 and connector 1712, through manifold 1720 and/or pump 1722, and through connector 1725 via a first fluid path into container 1732 (optionally through filter 1728), while quencher can be transferred from container 1704 and connector 1714, through manifold 1720 and/or pump 1722, and through connector 1724 via a second fluid path into container 1732 (optionally through filter 1729). In some embodiments, an additional fluid path can be coupled to manifold 1720 for transfer of fluids to and from a container configured for mixing the PIC and diluent (e.g., dilution container). In some embodiments, an additional fluid path can be coupled to manifold 1720, e.g., for transfer of fluids into a waste container or line. Processing set 1730 may be connected to connector 1724 via optional filter 1729 and to connector 1725 via optional filter 1728. Alternatively, or in addition, processing set 1730 may comprise a portion of fluid path (e.g., tubing, with branch, with Y-junction) which comprises optional filters 1728 and 1729, and the fluid path may be connected to connectors 1724 and 1725. This allows for transferring a determined amount of PIC from the PIC container, quencher from the quencher container, diluent from the diluent container, and processing solution from the processing solution container into one or more containers of processing set 1730 in a sterile manner while keeping PIC and quencher separate. In this example, processing set 1730 comprises mixing container 1732, incubation container 1734, and storage container (e.g., container with additive solution) 1736. In some embodiments, mixing container 1732 contains a processing solution (e.g., prior to addition of the blood composition, PIC, quencher, and/or diluent). Connectors 1712, 1714, 1716, 1718, 1724, and 1725 can be the same type of connector of the present disclosure, or different types of connectors of the present disclosure. In some embodiments, device 1700 comprises a housing. Optional scale 1726 can be within or outside of the housing. Scale 1726 can be used to weigh a blood composition of the present disclosure. In some embodiments, the system and/or device of FIG. 17 may comprise control circuitry and a computer system (not depicted) substantially similar to the system and/or device described for FIG. 8.

Blood composition container 1740 comprises a blood composition of the present disclosure. Blood composition container 1740 can be coupled (e.g., sterile coupled) to processing set 1730, allowing for the blood composition to be transferred into processing set 1730 (e.g., before, during, or after coupling processing set 1720 to fluid path 1706 and/or transfer of the PIC, quencher, diluent, and/or processing solution into processing set 1730). Processing set 1730 includes mixing container 1732, incubation container 1734, and additive solution container 1736. After transfer (e.g., by sterile transfer) of the blood composition into container 1732 and mixing with PIC and quencher, the mixture can be transferred (e.g., by sterile transfer) to incubation container 1734 for incubation, followed by transfer (e.g., by sterile transfer) into additive solution in container 1736. In some embodiments, the blood composition comprises RBCs (e.g., an RBC or whole blood composition). In some embodiments, the PIC comprises a nucleic acid binding ligand of the present disclosure that is an intercalator, e.g., an acridine such as β-alanine, N-(acridin-9-yl), 2-[bis(2-chloroethyl)amino]ethyl ester. In some embodiments, the quencher comprises cysteine or a derivative of cysteine, e.g., a peptide of 3-6 amino acids, wherein at least one of the amino acids is cysteine, N-acetyl cysteine, or S-acetyl cysteine. In some embodiments, the quencher is glutathione or a pharmaceutically acceptable salt thereof, e.g., glutathione monosodium salt.

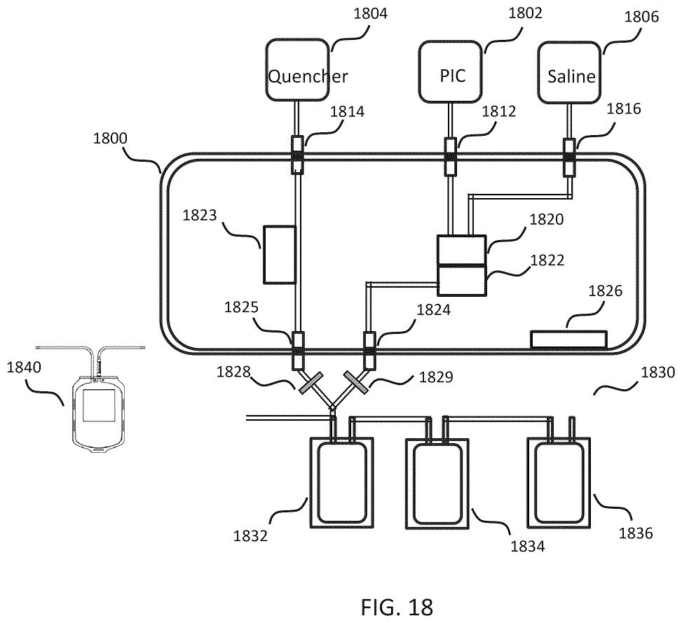

FIG. 18 depicts an exemplary system (e.g., dosing device system) for preparing a pathogen-inactivated blood composition, in accordance with some embodiments. The depicted system includes dosing device 1800 (e.g., a device comprising one or more components configured to control flow of one or more fluids of the present disclosure), one or more device-associated consumable components (e.g., reusable consumables) such as for example fluid path(s), connectors 1812, 1814, 1816, 1824 and/or 1825, and optional manifold (e.g., manifold/valve) 1820 and optional pumps 1822 and 1823; and processing set 1830 (e.g., a processing set of the present disclosure). In some embodiments, connectors 1812, 1814, 1816, 1824 and/or 1825 may be a portion of a connector, with the other portion of the connector a part of processing set 1830 and/or containers 1802, 1804, and/or 1806 (e.g., each a portion that is configured to join with the other portion). Device 1800 comprises a manifold (e.g., valve/manifold) 1820 and/or controller and/or actuator of manifold 1820; pumps 1822 and 1823 and/or controllers and/or actuators of pumps 1822 and 1823; and optional scale 1826. In this example, PIC container 1802 is coupled (e.g., reversibly, sterile coupled) to a fluid path (e.g., first fluid path) via connector 1812, which connects to the first fluid path, manifold 1820 and/or pump 1822 (connected to or otherwise associated with (e.g., loaded, mounted, attached, coupled)), and connector 1824 downstream. Quencher container 1804 is coupled (e.g., reversibly, sterile coupled) to a fluid path (e.g., second fluid path) via connector 1814, which connects to the second fluid path, pump 1823 (connected to or otherwise associated with (e.g., loaded, mounted, attached, coupled)), and connector 1825 downstream. This allows for delivery of a quencher of the present disclosure (e.g., glutathione or a pharmaceutically acceptable salt thereof) through a separate fluid path. Diluent container 1806 is coupled (e.g., reversibly, sterile coupled) to the first fluid path via connector 1816, which connects to the first fluid path, manifold 1820 and/or pump 1822 (connected to or otherwise associated with (e.g., loaded, mounted, attached, coupled)), and connector 1824 downstream. This allows for delivery of a diluent of the present disclosure (e.g., saline solution). In some embodiments, connectors 1812 and 1816 are connected to separate fluid paths into manifold 1820 and/or pump 1822. Pump 1822 and manifold 1820 control flow of fluid (e.g., comprising a PIC and/or diluent of the present disclosure) through the first fluid path, e.g., from PIC container 1802 through connector 1812 into the first fluid path, and from diluent container 1806 through connector 1816 into the first fluid path, and subsequently through connector 1824 into processing set 1830 (specifically, into mixing container 1832) optionally through filter 1829. In some embodiments, manifold 1820 can independently control fluid from container 1802 (e.g., comprising a PIC), and fluid from container 1806 (e.g., comprising a diluent) and/or can keep the PIC and diluent separate (e.g., prior to mixing). Pump 1823 controls flow of fluid (e.g., comprising a quencher of the present disclosure) through the second fluid path, e.g., from quencher container 1804 through connector 1814 into the second fluid path, and subsequently through connector 1825 into processing set 1830 (specifically, into mixing container 1832) optionally through filter 1828. This configuration can allow for the PIC/diluent and quencher to be transferred in separate fluid paths, e.g., prior to mixing in container 1832. In some embodiments, an additional fluid path can be coupled to manifold 1820 for transfer of fluids to and from a container configured for mixing the PIC and diluent (e.g., dilution container). In some embodiments, an additional fluid path can be coupled to manifold 1820, pump 1822, and/or pump 1823, e.g., for transfer of fluids into a waste container or line. Processing set 1830 may be connected to connector 1824 via optional filter 1829 and to connector 1825 via optional filter 1828. This allows for transferring a determined amount of PIC from the PIC container, quencher from the quencher container, and diluent from the diluent container into one or more containers of processing set 1830 in a sterile manner while keeping PIC/diluent and quencher separate. Alternatively, or in addition, processing set 1830 may comprise a portion of fluid path (e.g., tubing, with branch, with Y-junction) which comprises optional filters 1828 and 1829, and the fluid path may be connected to connectors 1824 and 1825. In this example, processing set 1830 comprises mixing container 1832, incubation container 1834, and storage container (e.g., container with additive solution) 1836. In some embodiments, mixing container 1832 contains a processing solution (e.g., prior to addition of the blood composition, PIC, quencher, and/or diluent). Connectors 1812, 1814, 1816, 1824, and 1825 can be the same type of connector of the present disclosure, or different types of connectors. In some embodiments, device 1800 comprises a housing. Optional scale 1826 can be within or outside of the housing. Scale 1826 can be used to weigh a blood composition of the present disclosure. In some embodiments, the system and/or device of FIG. 18 may comprise control circuitry and a computer system (not depicted) substantially similar to the system and/or device described for FIG. 8.

Blood composition container 1840 comprises a blood composition of the present disclosure. Blood composition container 1840 can be coupled (e.g., sterile coupled) to processing set 1830, allowing for the blood composition to be transferred into processing set 1830 (e.g., before, during, or after coupling processing set 1820 to fluid path 1806 and/or transfer of the PIC, quencher, and/or diluent into processing set 1830). Processing set 1830 includes mixing container 1832, incubation container 1834, and additive solution container 1836. After transfer (e.g., by sterile transfer) of the blood composition into container 1832 and mixing with PIC and quencher, the mixture can be transferred (e.g., by sterile transfer) to incubation container 1834 for incubation, followed by transfer (e.g., by sterile transfer) into additive solution in container 1836. In some embodiments, the blood composition comprises RBCs (e.g., an RBC or whole blood composition). In some embodiments, the PIC comprises a nucleic acid binding ligand of the present disclosure that is an intercalator, e.g., an acridine such as β-alanine, N-(acridin-9-yl), 2-[bis(2-chloroethyl)amino]ethyl ester. In some embodiments, the quencher comprises cysteine or a derivative of cysteine, e.g., a peptide of 3-6 amino acids, wherein at least one of the amino acids is cysteine, N-acetyl cysteine, or S-acetyl cysteine. In some embodiments, the quencher is glutathione or a pharmaceutically acceptable salt thereof, e.g., glutathione monosodium salt.

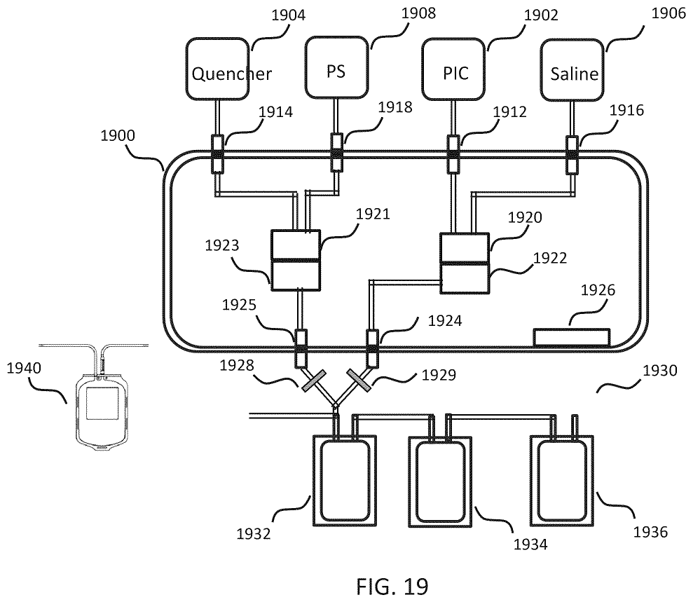

FIG. 19 depicts an exemplary system (e.g., dosing device system) for preparing a pathogen-inactivated blood composition, in accordance with some embodiments. The depicted system includes dosing device 1900 (e.g., a device comprising one or more components configured to control flow of one or more fluids of the present disclosure); one or more device-associated consumable components (e.g., reusable consumables) such as for example fluid path(s), connectors 1912, 1914, 1916, 1918, 1924 and/or 1925, and optional manifolds (e.g., manifold/valve) 1920 and 1921 and optional pumps 1922 and 1923; and processing set 1930 (e.g., a processing set of the present disclosure). In some embodiments, connectors 1912, 1914, 1916, 1918, 1924 and/or 1925 may be a portion of a connector, with the other portion of the connector a part of processing set 1930 and/or a bulk fluid container 1902, 1904, 1906, and/or 1908 (e.g., each a portion that is configured to join with the other portion). Device 1900 comprises manifolds (e.g., valve/manifolds) 1920 and 1921 and/or controllers and/or actuators of manifolds 1920 and 1921; pumps 1922 and 1923 and/or controllers and/or actuators of pumps 1922 and 1923; and optional scale 1926. In this example, PIC container 1902 is coupled (e.g., reversibly, sterile coupled) to a fluid path (e.g., first fluid path) via connector 1912, which connects to the first fluid path, manifold 1920 and/or pump 1922 (connected to or otherwise associated with (e.g., loaded, mounted, attached, coupled)), and connector 1924 downstream. Quencher container 1904 is coupled (e.g., reversibly, sterile coupled) to a fluid path (e.g., second fluid path) via connector 1914, which connects to the second fluid path, manifold 1921 and/or pump 1923 (connected to or otherwise associated with (e.g., loaded, mounted, attached, coupled)), and connector 1925 downstream. This allows for delivery of a quencher of the present disclosure (e.g., glutathione or a pharmaceutically acceptable salt thereof) through a separate fluid path. Diluent container 1906 is coupled (e.g., reversibly, sterile coupled) to a fluid path (e.g., the first fluid path) via connector 1916, which connects to the first fluid path, manifold 1920 and/or pump 1922 (connected to or otherwise associated with (e.g., loaded, mounted, attached, coupled)), and connector 1924 downstream. This allows for delivery of a diluent of the present disclosure (e.g., saline solution). Processing solution container 1908 is coupled (e.g., reversibly, sterile coupled) to a fluid path (e.g., the second fluid path) via connector 1918, which connects to the second fluid path, manifold 1921 and/or pump 1923 (connected to or otherwise associated with (e.g., loaded, mounted, attached, coupled)), and connector 1925 downstream. This allows for delivery of a processing solution of the present disclosure. In some embodiments, connectors 1912 and 1916 are connected to separate fluid paths to manifold 1920 and/or pump 1922. Pump 1922 and manifold 1920 control flow of fluid (e.g., comprising a PIC and/or diluent of the present disclosure) through the first fluid path, e.g., from PIC container 1902 through connector 1912 into the first fluid path, and from diluent container 1906 through connector 1916 into the first fluid path, and subsequently through connector 1924 into processing set 1930 (specifically, into mixing container 1932) optionally through filter 1929. In some embodiments, manifold 1920 can independently control fluid from container 1902 (e.g., comprising a PIC), and fluid from container 1906 (e.g., comprising a diluent) and/or can keep the PIC and diluent separate (e.g., prior to mixing in container 1932, prior to mixing in a dilution container). In some embodiments, connectors 1914 and 1918 are connected to separate fluid paths to manifold 1921 and/or pump 1923. Pump 1923 and manifold 1921 control flow of fluid (e.g., comprising a quencher and/or processing solution of the present disclosure) through the second fluid path, e.g., from quencher container 1904 through connector 1914 into the second fluid path, and from processing solution container 1908 through connector 1918 into the second fluid path, and subsequently through connector 1925 into processing set 1930 (specifically, into mixing container 1932) optionally through filter 1928. In some embodiments, manifold 1921 can independently control fluid from container 1904 (e.g., comprising a quencher), and fluid from container 1908 (e.g., comprising a processing solution) and/or can keep the quencher and processing solution separate (e.g., prior to mixing in container 1932). This configuration can allow for the PIC/diluent and quencher/processing solution to be transferred in separate fluid paths, e.g., prior to mixing in container 1932. In some embodiments, an additional fluid path can be coupled to manifold 1921 for transfer of fluids to and from a container configured for mixing the PIC and diluent (e.g., dilution container). In some embodiments, an additional fluid path can be coupled to valve/manifold 1920 and/or 1921, e.g., for transfer of fluids into a waste container or line. Processing set 1930 is connected to connector 1924 via optional filter 1929 and to connector 1925 via optional filter 1928. This allows for transferring a determined amount of PIC from the PIC container, quencher from the quencher container, diluent from the diluent container, and processing solution from the processing solution container into one or more containers of processing set 1930 in a sterile manner while keeping PIC/diluent and quencher/processing solution separate. Alternatively, or in addition, processing set 1930 may comprise a portion of fluid path (e.g., tubing, with branch, with Y-junction) which comprises optional filters 1928 and 1929, and the fluid path may be connected to connectors 1924 and 1925. In this example, processing set 1930 comprises mixing container 1932, incubation container 1934, and storage container (e.g., container with additive solution) 1936. Connectors 1912, 1914, 1916, 1918, 1924, and 1925 can be the same type of connector of the present disclosure, or different types of connectors of the present disclosure. In some embodiments, device 1900 comprises a housing. Optional scale 1926 can be within or outside of the housing. Scale 1926 can be used to weigh a blood composition of the present disclosure. In some embodiments, the system and/or device of FIG. 19 may comprise control circuitry and a computer system (not depicted) substantially similar to the system and/or device described for FIG. 8.

Blood composition container 1940 comprises a blood composition of the present disclosure. Blood composition container 1940 can be coupled (e.g., sterile coupled) to processing set 1930, allowing for the blood composition to be transferred into processing set 1930 (e.g., before, during, or after coupling processing set 1920 to fluid path 1906 and/or transfer of the PIC, quencher, diluent, and/or processing solution into processing set 1930). Processing set 1930 includes mixing container 1932, incubation container 1934, and additive solution container 1936. After transfer (e.g., by sterile transfer) of the blood composition into container 1932 and mixing with PIC and quencher, the mixture can be transferred (e.g., by sterile transfer) to incubation container 1934 for incubation, followed by transfer (e.g., by sterile transfer) into additive solution in container 1936. In some embodiments, the blood composition comprises RBCs (e.g., an RBC or whole blood composition). In some embodiments, the PIC comprises a nucleic acid binding ligand of the present disclosure that is an intercalator, e.g., an acridine such as β-alanine, N-(acridin-9-yl), 2-[bis(2-chloroethyl)amino]ethyl ester. In some embodiments, the quencher comprises cysteine or a derivative of cysteine, e.g., a peptide of 3-6 amino acids, wherein at least one of the amino acids is cysteine, N-acetyl cysteine, or S-acetyl cysteine. In some embodiments, the quencher is glutathione or a pharmaceutically acceptable salt thereof, e.g., glutathione monosodium salt.

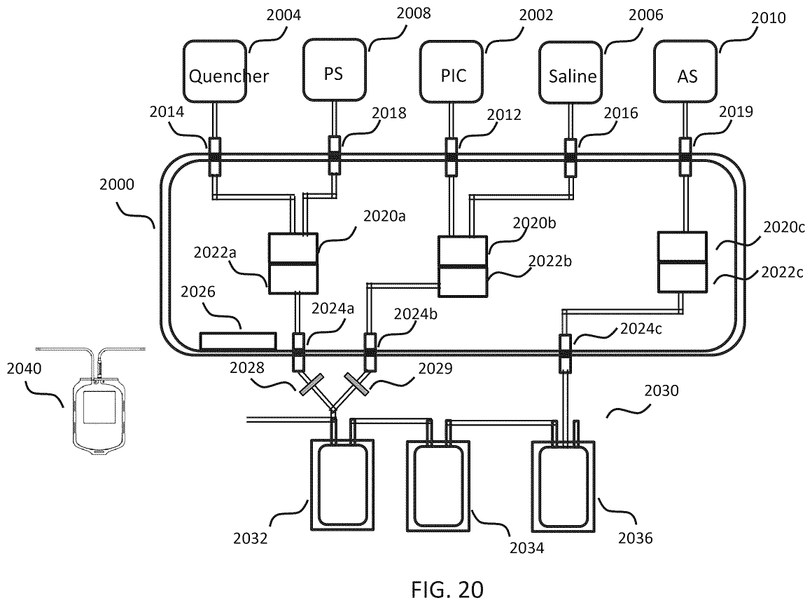

FIG. 20 depicts an exemplary system (e.g., dosing device system) for preparing a pathogen-inactivated blood composition, in accordance with some embodiments. The depicted system includes dosing device 2000 (e.g., a device comprising one or more components configured to control flow of one or more fluids of the present disclosure); one or more device-associated consumable components (e.g., reusable consumables) such as for example fluid path(s), connectors 2012, 2014, 2016, 2018, 2019, 2024a, 2024b and/or 2024c, and optional manifolds (e.g., manifold/valve) 2020a, 2020b and 2020c and optional pumps 2022a, 2020b and 2022c and processing set 2030 (e.g., a processing set of the present disclosure). In some embodiments, connectors 2012, 2014, 2016, 2018, 2019, 2024a, 2024b and/or 2024c may be a portion of a connector, with the other portion of the connector a part of processing set 2030 and/or a bulk fluid container 2002, 2004, 2006, 2008, and/or 2010 (e.g., each a portion that is configured to join with the other portion). Device 2000 comprises manifolds (e.g., valve/manifolds) 2020a, 2020b, and 2020c and/or controllers and/or actuators of manifolds 2020a, 2020b and 2020c; pumps 2022a, 2022b, and 2022c and/or controllers and/or actuators of pumps 2022a, 2022b and 2022c; and optional scale 2026. In this example, PIC container 2002 is coupled (e.g., reversibly, sterile coupled) to a fluid path (e.g., first fluid path) via connector 2012, which connects to the first fluid path, manifold 2020b and/or pump 2022b (connected to or otherwise associated with (e.g., loaded, mounted, attached, coupled)), and connector 2024b downstream. Quencher container 2004 is coupled (e.g., reversibly, sterile coupled) to a fluid path (e.g., second fluid path) via connector 2014, which connects to the second fluid path, manifold 2020a and/or pump 2022a (connected to or otherwise associated with (e.g., loaded, mounted, attached, coupled)), and connector 2024a downstream. This allows for delivery of a quencher of the present disclosure (e.g., glutathione or a pharmaceutically acceptable salt thereof) through a separate fluid path. Diluent container 2006 is coupled (e.g., reversibly, sterile coupled) to a fluid path (e.g., the first fluid path) via connector 2016, which connects to the first fluid path, manifold 2020b and/or pump 2022b (connected to or otherwise associated with (e.g., loaded, mounted, attached, coupled)), and connector 2024b downstream. This allows for delivery of a diluent of the present disclosure (e.g., saline solution). Processing solution container 2008 is coupled (e.g., reversibly, sterile coupled) to a fluid path (e.g., the second fluid path) via connector 2018, which connects to the second fluid path, /manifold 2020a and/or pump 2022a (connected to or otherwise associated with (e.g., loaded, mounted, attached, coupled)), and connector 2024a downstream. This allows for delivery of a processing solution of the present disclosure. Additive solution container 2010 is coupled (e.g., reversibly, sterile coupled) to a fluid path (e.g., a third fluid path) via connector 2019, which connects to the third fluid path, manifold 2020c and/or pump 2022c (connected to or otherwise associated with (e.g., loaded, mounted, attached, coupled)), and connector 2024c downstream. In some embodiments, connectors 2012 and 2016 are connected to separate fluid paths to manifold 2020b and/or pump 2022b. Pump 2020b and manifold 2022b control flow of fluid (e.g., comprising a PIC and/or diluent of the present disclosure) through the first fluid path, e.g., from PIC container 2002 through connector 2012 into the first fluid path, and from diluent container 2006 through connector 2016 into the first fluid path, and subsequently through connector 2024b into processing set 2030 (specifically, into mixing container 2032) optionally through filter 2029. In some embodiments, manifold 2020b can independently control fluid from container 2002 (e.g., comprising a PIC), and fluid from container 2006 (e.g., comprising a diluent) and/or can keep the PIC and diluent separate (e.g., prior to mixing in container 2032, prior to mixing in a dilution container). In some embodiments, connectors 2014 and 2018 are connected to separate fluid paths to manifold 2020a and/or pump 2022a. Pump 2022a and manifold 2020a control flow of fluid (e.g., comprising a quencher and/or processing solution of the present disclosure) through the second fluid path, e.g., from quencher container 2004 through connector 2014 into the second fluid path, and from processing solution container 2008 through connector 2018 into the second fluid path, and subsequently through connector 2024a into processing set 2030 (specifically, into mixing container 2032) optionally through filter 2028. In some embodiments, manifold 2020a can independently control fluid from container 2004 (e.g., comprising a quencher), and fluid from container 2008 (e.g., comprising a processing solution) and/or can keep the quencher and processing solution separate (e.g., prior to mixing in container 2032). This configuration can allow for the PIC/diluent and quencher/processing solution to be transferred in separate fluid paths, e.g., prior to mixing in container 2032. Pump 2022c and manifold 2020c control flow of fluid (e.g., comprising an additive solution of the present disclosure) through the third fluid path, e.g., from additive solution container 2010 through connector 2019 into the third fluid path, and subsequently through connector 2024c into processing set 2030 (specifically, into storage container 2036). In some embodiments, an additional fluid path can be coupled to manifold 2020b for transfer of fluids to and from a container configured for mixing the PIC and diluent (e.g., dilution container). In some embodiments, an additional fluid path can be coupled to manifold 2020a, 2020b, and/or 2020c, e.g., for transfer of fluids into a waste container or line. Processing set 2030 is connected to connector 2024b via optional filter 2029, to connector 2024a via optional filter 2028, and to connector 2024c. This allows for transferring a determined amount of PIC from the PIC container, quencher from the quencher container, diluent from the diluent container, processing solution from the processing solution container, and additive solution from the additive solution container into one or more containers of processing set 2030 in a sterile manner while keeping PIC/diluent, quencher/processing solution, and additive solution separate. Alternatively, or in addition, processing set 2030 may comprise a portion of fluid path (e.g., tubing, with branch, with Y-junction) which comprises optional filters 2028 and 2029, and the fluid path may be connected to connectors 2024a and 2024b. In this example, processing set 2030 comprises mixing container 2032, incubation container 2034, and storage container (e.g, container for additive solution) 2036. Connectors 2012, 2014, 2016, 2018, 2019, 2024a, 2024b, and 2024c can be the same type of connector of the present disclosure, or different types of connectors of the present disclosure. In some embodiments, device 2000 comprises a housing. Optional scale 2026 can be within or outside of the housing. Scale 2026 can be used to weigh a blood composition of the present disclosure. In some embodiments, the system and/or device of FIG. 20 may comprise control circuitry and a computer system (not depicted) substantially similar to the system and/or device described for FIG. 8.

Blood composition container 2040 comprises a blood composition of the present disclosure. Blood composition container 2040 can be coupled (e.g., sterile coupled) to processing set 2030, allowing for the blood composition to be transferred into processing set 2030 (e.g., before, during, or after coupling processing set 2020 to fluid path 2006 and/or transfer of the PIC, quencher, diluent, processing solution, and/or additive solution into processing set 2030). Processing set 2030 includes mixing container 2032, incubation container 2034, and additive solution container 2036. After transfer (e.g., by sterile transfer) of the blood composition into container 2032 and mixing with PIC and quencher, the mixture can be transferred (e.g., by sterile transfer) to incubation container 2034 for incubation, followed by transfer (e.g., by sterile transfer) into additive solution in container 2036. In some embodiments, the blood composition comprises RBCs (e.g., an RBC or whole blood composition). In some embodiments, the PIC comprises a nucleic acid binding ligand of the present disclosure that is an intercalator, e.g., an acridine such as β-alanine, N-(acridin-9-yl), 2-[bis(2-chloroethyl)amino]ethyl ester. In some embodiments, the quencher comprises cysteine or a derivative of cysteine, e.g., a peptide of 3-6 amino acids, wherein at least one of the amino acids is cysteine, N-acetyl cysteine, or S-acetyl cysteine. In some embodiments, the quencher is glutathione or a pharmaceutically acceptable salt thereof, e.g., glutathione monosodium salt.

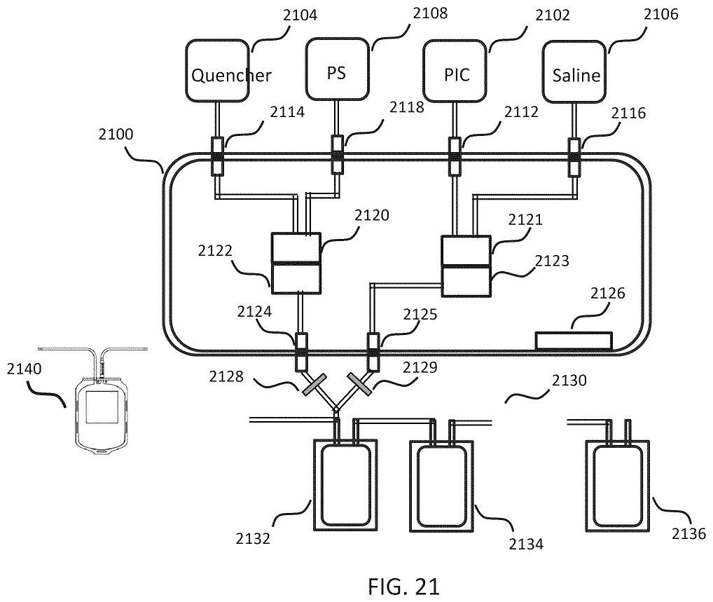

FIG. 21 depicts an exemplary system (e.g., dosing device system) for preparing a pathogen-inactivated blood composition, in accordance with some embodiments. The depicted system includes dosing device 2100 (e.g., a device comprising one or more components configured to control flow of one or more fluids of the present disclosure); one or more device-associated consumable components (e.g., reusable consumables) such as for example fluid path(s), connectors 2112, 2114, 2116, 2118, 2124 and/or 2125, and optional manifolds (e.g., manifold/valve) 2120 and 2121 and optional pumps 2122 and 2123 and processing set 2130 (e.g., a processing set of the present disclosure). In some embodiments, connectors 2112, 2114, 2116, 2118, 2124 and/or 2125 may be a portion of a connector, with the other portion of the connector a part of processing set 2130 and/or a bulk fluid container 2102, 2104, 2106, and/or 2108 (e.g., each a portion that is configured to join with the other portion). Device 2100 comprises manifolds (e.g., valve/manifolds) 2120 and 2121 and/or controllers and/or actuators for manifolds 2120 and 2121; pumps 2122 and 2123 and/or controllers and/or actuators for pumps 2122 and 2123; and optional scale 2126. In this example, PIC container 2102 is coupled (e.g., reversibly, sterile coupled) to a fluid path (e.g., first fluid path) via connector 2112, which connects to the first fluid path, manifold 2121 and/or pump 2123 (connected to or otherwise associated with (e.g., loaded, mounted, attached, coupled)), and connector 2125 downstream. Quencher container 2104 is coupled (e.g., reversibly, sterile coupled) to a fluid path (e.g., second fluid path) via connector 2114, which connects to the second fluid path, manifold 2120 and/or pump 2122 (connected to or otherwise associated with (e.g., loaded, mounted, attached, coupled)), and connector 2124 downstream. This allows for delivery of a quencher of the present disclosure (e.g., glutathione or a pharmaceutically acceptable salt thereof) through a separate fluid path. Diluent container 2106 is coupled (e.g., reversibly, sterile coupled) to a fluid path (e.g., the first fluid path) via connector 2116, which connects to the first fluid path, manifold 2121 and/or pump 2123 (connected to or otherwise associated with (e.g., loaded, mounted, attached, coupled)), and connector 2125 downstream. This allows for delivery of a diluent of the present disclosure (e.g., saline solution). Processing solution container 2108 is coupled (e.g., reversibly, sterile coupled) to a fluid path (e.g., the second fluid path) via connector 2118, which connects to the second fluid path, manifold 2120 and/or pump 2122 (connected to or otherwise associated with (e.g., loaded, mounted, attached, coupled)), and connector 2124 downstream. This allows for delivery of a processing solution of the present disclosure. In some embodiments, connectors 2112 and 2116 are connected to separate fluid paths to manifold 2121 and/or pump 2123. Pump 2123 and manifold 2121 control flow of fluid (e.g., comprising a PIC and/or diluent of the present disclosure) through the first fluid path, e.g., from PIC container 2102 through connector 2112 into the first fluid path, and from diluent container 2106 through connector 2116 into the first fluid path, and subsequently through connector 2125 into processing set 2130 (specifically, into mixing container 2132) optionally through filter 2129. In some embodiments, manifold 2121 can independently control fluid from container 2102 (e.g., comprising a PIC), and fluid from container 2106 (e.g., comprising a diluent) and/or can keep the PIC and diluent separate (e.g., prior to mixing in container 2132, prior to mixing in a dilution container). In some embodiments, connectors 2114 and 2118 are connected to separate fluid paths into manifold 2120 and/or pump 2122. Pump 2122 and manifold 2120 control flow of fluid (e.g., comprising a quencher and/or processing solution of the present disclosure) through the second fluid path, e.g., from quencher container 2104 through connector 2114 into the second fluid path, and from processing solution container 2108 through connector 2118 into the second fluid path, and subsequently through connector 2124 into processing set 2130 (specifically, into mixing container 2132) optionally through filter 2128. In some embodiments, manifold 2120 can independently control fluid from container 2104 (e.g., comprising a quencher), and fluid from container 2108 (e.g., comprising a processing solution) and/or can keep the quencher and processing solution separate (e.g., prior to mixing in container 2132). This configuration can allow for the PIC/diluent and quencher/processing solution to be transferred in separate fluid paths, e.g., prior to mixing in container 2132. In some embodiments, an additional fluid path can be coupled to manifold 2021 for transfer of fluids to and from a container configured for mixing the PIC and diluent (e.g., dilution container). In some embodiments, an additional fluid path can be coupled to valve/manifold 2120 and/or valve/manifold 2121, e.g., for transfer of fluids into a waste container or line. Processing set 2130 is connected to connector 2124 via optional filter 2128 and to connector 2125 via optional filter 2129. This allows for transferring a determined amount of PIC from the PIC container, quencher from the quencher container, diluent from the diluent container, and processing solution from the processing solution container into one or more containers of processing set 2130 in a sterile manner while keeping PIC/diluent and quencher/processing solution separate. Alternatively, or in addition, processing set 2130 may comprise a portion of fluid path (e.g., tubing, with branch, with Y-junction) which comprises optional filters 2128 and 2129, and the fluid path may be connected to connectors 2124 and 2125. In this example, processing set 2130 comprises mixing container 2132 and incubation container 2134. Additive solution can be supplied in separate storage container (e.g., container with additive solution) 2136, which can be coupled to processing set 2130 (e.g., to incubation container 2134). Connectors 2112, 2114, 2116, 2118, 2124, and 2125 can be the same type of connector of the present disclosure, or different types of connectors of the present disclosure. In some embodiments, device 2100 comprises a housing. Optional scale 2126 can be within or outside of the housing. Scale 2126 can be used to weigh a blood composition of the present disclosure. In some embodiments, the system and/or device of FIG. 21 may comprise control circuitry and a computer system (not depicted) substantially similar to the system and/or device described for FIG. 8.

Blood composition container 2140 comprises a blood composition of the present disclosure. Blood composition container 2140 can be coupled (e.g., sterile coupled) to processing set 2130, allowing for the blood composition to be transferred into processing set 2130 (e.g., before, during, or after coupling processing set 2120 to fluid path 2106 and/or transfer of the PIC, quencher, diluent, and/or processing solution into processing set 2130). Processing set 2130 includes mixing container 2132, incubation container 2134, and additive solution container 2136. After transfer (e.g., by sterile transfer) of the blood composition into container 2132 and mixing with PIC and quencher, the mixture can be transferred (e.g., by sterile transfer) to incubation container 2134 for incubation, followed by transfer (e.g., by sterile transfer) into additive solution in container 2136. In some embodiments, the blood composition comprises RBCs (e.g., an RBC or whole blood composition). In some embodiments, the PIC comprises a nucleic acid binding ligand of the present disclosure that is an intercalator, e.g., an acridine such as β-alanine, N-(acridin-9-yl), 2-[bis(2-chloroethyl)amino]ethyl ester. In some embodiments, the quencher comprises cysteine or a derivative of cysteine, e.g., a peptide of 3-6 amino acids, wherein at least one of the amino acids is cysteine, N-acetyl cysteine, or S-acetyl cysteine. In some embodiments, the quencher is glutathione or a pharmaceutically acceptable salt thereof, e.g., glutathione monosodium salt.

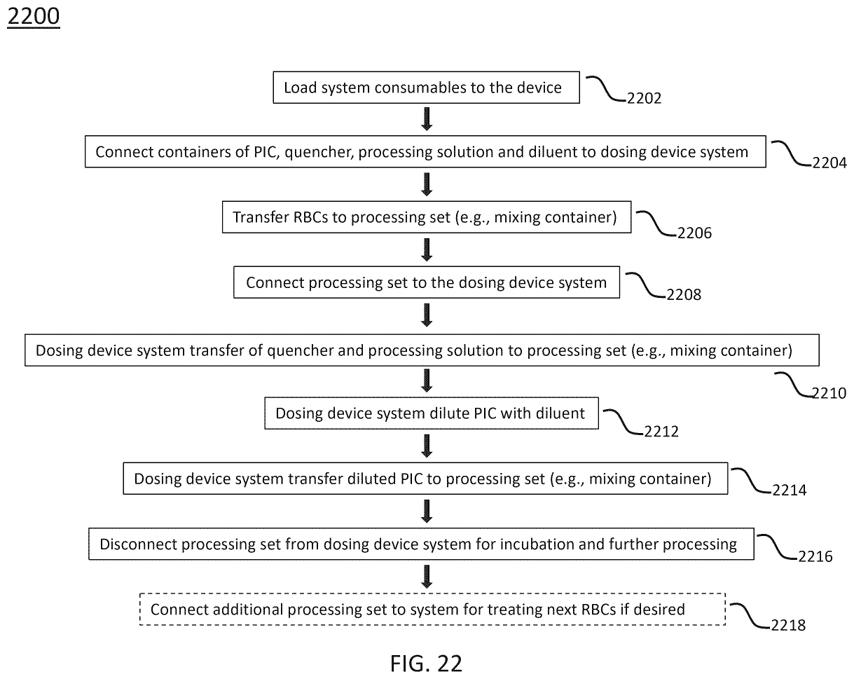

FIG. 22 depicts a flowchart of an exemplary and non-limiting method 2200 for preparing a pathogen inactivated blood composition, according to some embodiments. At block 2202, system consumable(s) (e.g., device-associated consumable(s)) are loaded to the device. System consumable(s) refers to reusable consumable component(s) of the system (e.g., associated with the device) comprising one or more fluid paths, connectors, manifolds and in certain embodiments pumps, and which are contemplated to be used for the dosing of multiple processing sets. Any of the devices of the present disclosure can be used in the methods herein. At block 2204, a PIC container (e.g., containing reconstituted PIC) of the present disclosure, quencher container (e.g., containing reconstituted PIC) of the present disclosure, and processing solution container of the present disclosure are connected to a dosing device system of the present disclosure. At block 2206, a blood composition comprising RBCs of the present disclosure is transferred (e.g., via sterile coupling) to a processing set of the present disclosure (e.g., from a blood composition container and into a mixing container as described herein). At block 2208, the processing set (e.g., via the mixing container) is connected to the dosing device system. At block 2210, the dosing device system controls fluid flow such that quencher (e.g., from a quencher container as described herein) and processing solution (e.g., from a processing solution container as described herein) are transferred to the processing set (e.g., into the mixing container). At block 2212, the dosing device system controls fluid flow such that the PIC (e.g., from a PIC container as described herein) is diluted with diluent (e.g., from a diluent container as described herein), such as for example in another container component of the dosing device system. At block 2214, the dosing device system controls fluid flow such that the diluted PIC is transferred to the processing set (e.g., into the mixing container). At block 2216, the processing set is disconnected from the dosing device system. Subsequently, the blood composition in admixture with the PIC and quencher can be incubated (e.g., upon transfer to an incubation container of the present disclosure) and optionally further processed (e.g., exchange fluid to additive solution). Optionally, at block 2218, a second processing set can be connected to the device, e.g., for treating a second blood composition, and the method can be repeated. This allows for higher throughput of blood composition processing, e.g., using a device of the present disclosure. In method 2200, some blocks can, optionally, be combined, the order of some blocks can, optionally, be changed, and some blocks can, optionally, be omitted. In some examples, additional steps can be performed in combination with the method 2200. Accordingly, the operations as illustrated (and described in greater detail herein) are exemplary by nature and, as such, should not be viewed as limiting.

In some embodiments, the PIC (e.g., β-alanine, N-(acridin-9-yl), 2-[bis(2-chloroethyl)amino]ethyl ester) and/or quencher (e.g., glutathione) does/do not require dilution prior to use (e.g., prior to transferring into a mixing container as disclosed herein). In some embodiments, the PIC and/or quencher is diluted (e.g., in a diluent as disclosed herein) prior to use (e.g., prior to transferring into a mixing container as disclosed herein). In some embodiments, dilution of the PIC and/or quencher is performed prior to coupling the PIC container and/or quencher container, respectively, to a fluid path of the system. In some embodiments, a dilution of the PIC and/or quencher is performed after coupling the PIC container and/or quencher container, respectively, to a fluid path of the system. For example, a system of the present disclosure may be configured to perform a dilution of the PIC and/or quencher. In one embodiment, the system is configured to (i) transfer an amount of diluent from a diluent container into the PIC container (e.g., to provide a diluted PIC solution); and (ii) transfer an amount of the mixture of diluent and the PIC (e.g., diluted PIC solution) into the mixing container (e.g., to provide a determined amount of PIC into the mixing container), such as for example by way of a first fluid path. In another embodiment, the system is configured to (i) transfer an amount of PIC from the PIC container into a diluent container (e.g., to provide a diluted PIC solution); and (ii) transfer an amount of the mixture of diluent and the PIC (e.g., diluted PIC solution) into the mixing container (e.g., to provide the determined amount of the PIC into the mixing container), such as for example by way of a first fluid path. In another embodiment, the system is configured to (i) transfer an amount diluent from a diluent container into a container for dilution (e.g., dilution container, third container), (ii) transfer an amount of PIC from the PIC container into the container for dilution; and (iii) transfer an amount of the mixture of diluent and the PIC (e.g., diluted PIC solution) into the mixing container (e.g., to provide the determined amount of the PIC into the mixing container), such as for example by way of a first fluid path, wherein the first fluid path comprises the container for dilution.

A variety of components configured to control flow of fluids are contemplated for use with a system and/or device as described herein. In some embodiments, the component(s) comprise a pump. Pumps contemplated for use herein include, without limitation, centrifugal or positive displacement pumps, positive displacement metering pump, rotary or reciprocating pumps. Rotary pumps can include for example, gear, lobe, vane, and roller (peristaltic) pumps. Reciprocating pumps can include for example, diaphragm, piston, syringe, and bellows pumps. In some embodiments, a pump controller and/or pump actuator of a device of the present application may control (e.g., by computer, processor, control circuitry, mechanical, motor, data, signal) a pump that is part of (e.g., component of) a device-associated consumable (e.g., reusable consumable) of a system. In some embodiments, the component(s) comprise a press, e.g., to facilitate transfer of the blood composition or the PIC and/or quencher into the mixing container by applying pressure to the blood composition container or PIC container, respectively. In some embodiments, the component(s) comprise a manifold. For example, one or more sterile tubing(s) (e.g., part of a fluid path of the present disclosure) can be connected to the manifold and thus connect one or more of the mixing container, PIC container, optional quencher container, optional additive or diluent container, and/or blood composition container. In some embodiments, the PIC container is connected to the manifold via a first segment of the first fluid path, and the manifold is connected to the mixing container via a second segment of the first fluid path. For example, by “connected to,” the PIC container may be reversibly coupled to the first segment of the first fluid path, which is in turn connected (e.g., fixedly connected, not reversibly coupled) to a manifold. Likewise, the mixing container may be reversibly coupled to the second segment of the first fluid path, which in turn is connected (e.g., fixedly connected, not reversibly coupled) to a manifold. In some embodiments, a quencher container is connected to the manifold via a fluid path (e.g., a second or third fluid path connecting a quencher container and a mixing container of the present disclosure) and such fluid path may comprise first and/or second segments similar to that described above for the PIC container and mixing container. In some embodiments, a second fluid path segment connecting the mixing container and manifold may be shared by (e.g., used for both) the PIC and quencher. In some embodiments, a second fluid path segment connecting the mixing container and manifold and used for the PIC may be a different fluid path than the second fluid path segment connecting the mixing container and manifold and used for the quencher. In some embodiments, such different fluid paths (e.g., second fluid path segments) may be joined (e.g., by way of a branch connector or Y-junction) at or in proximity to a coupling (e.g., reversible coupling, sterile coupling) for coupling the fluid path(s) to the mixing container. In some embodiments, the blood composition container is connected to a fluid path. For example, the second segment of the first fluid path can comprise a Y-junction that is also connected to the blood composition container. In some embodiments, a manifold of the present disclosure can be operably linked to an air filter, e.g., to provide sterile air used to control flow of fluids. As used herein, a manifold may generally refer to a component that manages (e.g., regulates) the control and distribution of fluids, such as by opening and/or closing the flow of fluid for one or more fluid paths (e.g., channels) (e.g., from a fluid path, to a fluid path, within a fluid path, at a fluid path junction), combining multiple fluid paths and/or dividing fluid path(s) into multiple fluid paths, such as for example using one or more valves or other control mechanisms of the manifold. In some embodiments, a manifold controller and/or manifold actuator of a device of the present application may control (e.g., by computer, processor, control circuitry, mechanical, motor, data, signal) a manifold that is part of (e.g., component of) a device-associated consumable (e.g., reusable consumable) of a system. A manifold may also be referred to as a fluid control manifold, multi-valve manifold, flow control manifold, flow control hub or cassette, manifold assembly, manifold stopcock, and the like.

In some embodiments, a fluid path of the present disclosure comprises sterile tubing, such as for example tubing compatible with biological fluids (e.g., blood products). In some embodiments, the sterile tubing is configured for mounting onto the device. In some embodiments, a fluid path of the present disclosure comprises contiguous tubing configured at one end for coupling (e.g., removably coupling, sterile coupling) to a PIC container or a quencher container and at another end for coupling (e.g., removably coupling, sterile coupling) to a mixing container. In some embodiments, a fluid path of the present disclosure further comprises one or more of: a connector, a breakable connector, a cannula, a filter, a manifold, a container, a junction (e.g., Y-junction), and a coupling.

In some embodiments, a system of the present disclosure comprises both consumable (e.g., disposable) and permanent (e.g., built-in or pre-manufactured along with one or more mechanical system components) components. In some embodiments, a consumable component comprises a fluid path that may be used two or more times, five or more times, ten or more times, fifteen or more times, or twenty or more times. For example, the same consumable fluid path component may be used at a device for a certain period of time (e.g., for a day, week, month, etc.) and/or for a certain number of uses. In some embodiments, a fluid path of the present disclosure, e.g., the first fluid path, is configured for multiple uses. For example, the fluid path can be a disposable fluid path, configured for preparing more than one composition (e.g., sequentially), and/or configured for coupling and de-coupling the PIC container and/or quencher container and the mixing container. In some embodiments, the PIC container and/or the mixing container are configured for reversibly coupling to a fluid path, e.g., the first fluid path. In some embodiments, the quencher container is configured for reversibly coupling to a fluid path, e.g., the second or third fluid path.

In some embodiments, a device of the present disclosure further comprises one or more detectors (e.g., fluid detectors) configured to detect fluid transfer within (e.g., within, through, or in) a fluid path of the present disclosure. For example, in some embodiments, the device comprises one or more fluid detectors configured to detect fluid transfer within (e.g., within, through, or in) a first fluid path of the present disclosure, e.g., between a PIC container and a mixing container. For example, in some embodiments, the device comprises one or more fluid detectors configured to detect fluid transfer within (e.g., within, through, or in) a fluid path of the present disclosure connecting a blood composition container and a mixing container. In some embodiments, the one or more fluid detectors are configured to detect fluid transfer within (e.g., within, through, or in) a fluid path and cause the device (e.g., using one or more of the components configured to control flow of fluids) to terminate fluid transfer within (e.g., within, through, or in) the fluid path.

In some embodiments, a device of the present disclosure further comprises a scale. The scale can be used, e.g., to weigh a blood composition of the present disclosure (e.g., in a blood composition container) and/or to weigh a mixing container of the present disclosure during and/or after transfer of the determined amount of the PIC into the mixing container. In some embodiments, the scale can be used to weigh other containers (e.g., contents of the containers) as provided herein, such as for example a PIC container, a quencher container, a diluent or additive container, and/or a container for dilution of a PIC or quencher. In some embodiments, the scale is at an angle (e.g., supporting surface for weighing is at an angle). In some embodiments, the scale is predominantly horizontal, such as a platform scale. In some embodiments, the scale can be predominantly vertical, such as a hanging scale. In some embodiments, the scale can be used to admix a blood composition and the determined amount of the PIC in the mixing container, e.g., by agitation or rocking, such as agitation at a speed between 10 and 30 RPM or between 14 and 22 RPM and/or rocking at an angle of rocking between 20° and 70°, between 30° and 65°, or between 38° and 58°.

In some embodiments, a device of the present disclosure further comprises one or more supports configured to support the PIC container, the diluent or additive container, the quencher container, and/or the blood composition container.

In some embodiments, a device of the present disclosure further comprises one or more components configured to receive an input. For example, such component(s) can include, without limitation, a scanner (e.g., barcode scanner, QR code scanner, radio frequency identification (RFID) scanner), QR code scanner, radio frequency identification (RFID) scanner, touchscreen display, keyboard, mouse, and the like.

In some embodiments, a device of the present disclosure further comprises one or more processors, e.g., one or more central processing units (CPUs) or processor(s). Processor(s) may comprise at least one data processor for executing program components for executing user- or system-generated requests. A user may include a person, a person using a device such as those included in this disclosure, or such a device itself. The processor may include specialized processing units such as integrated system (bus) controllers, memory management control units, floating point units, graphics processing units, digital signal processing units, etc. The processor may include a microprocessor, such as AMD Athlon, Duron or Opteron, ARM's application, embedded or secure processors, IBM PowerPC, Intel's Core, Itanium, Xeon, Celeron or other line of processors, etc. The processor may be implemented using mainframe, distributed processor, multi-core, parallel, grid, or other architectures. Some embodiments may utilize embedded technologies like application-specific integrated circuits (ASICs), digital signal processors (DSPs), Field Programmable Gate Arrays (FPGAs), etc.

Processor(s) may be disposed in communication with one or more input/output (I/O) devices via I/O interface. An I/O interface may employ communication protocols/methods such as, without limitation, audio, analog, digital, monoaural, RCA, stereo, IEEE-1394, serial bus, universal serial bus (USB), infrared, PS/2, BNC, coaxial, component, composite, digital visual interface (DVI), high-definition multimedia interface (HDMI), RF antennas, S-Video, VGA, IEEE 802.11 a/b/g/n/x, Bluetooth, cellular (e.g., code-division multiple access (CDMA), high-speed packet access (HSPA+), global system for mobile communications (GSM), long-term evolution (LTE), WiMax, or the like), etc.

In some embodiments, a device of the present disclosure is connected (e.g., via wired or wireless connection) to one or more peripherals, including without limitation a display (e.g., a touchscreen display, barcode scanner, QR code scanner, RFID scanner, label printer, antenna, keyboard, mouse, joystick, (infrared) remote control, camera, card reader, fax machine, dongle, biometric reader, microphone, touch screen, touchpad, trackball, sensor (e.g., accelerometer, light sensor, GPS, gyroscope, proximity sensor, or the like), stylus, scanner, storage device, transceiver, video device/source, visors, electrical pointing devices, etc. For example, device 100 shown in FIG. 1A is connected to display 110 (e.g., a touchscreen display), barcode scanner 112, and label printer 114. In some embodiments, the peripheral(s) include one or more components for receiving an input, including without limitation a barcode scanner, QR code scanner, RFID scanner, keyboard, mouse, touchscreen display, and the like. In some embodiments, the peripheral(s) include one or more components for generating an output, including without limitation a printer, fax machine, video display (e.g., cathode ray tube (CRT), liquid crystal display (LCD), light-emitting diode (LED), plasma, or the like), audio speaker, etc.

In some embodiments, the device can comprise a transceiver, e.g., disposed in connection with the processor(s). The transceiver may facilitate various types of wireless transmission or reception. For example, the transceiver may include an antenna operatively connected to a transceiver chip (e.g., Texas Instruments WiLink WL1283, Broadcom BCM4750IUB8, Infineon Technologies X-Gold 618-PMB9800, or the like), providing IEEE 802.11a/b/g/n, Bluetooth, FM, global positioning system (GPS), 2G/3G HSDPA/HSUPA communications, etc. In some embodiments, processor(s) may be disposed in communication with a communication network via a network interface. A network interface may communicate with a communication network and may employ connection protocols including, without limitation, direct connect, Ethernet (e.g., twisted pair 10/100/1000 Base T), transmission control protocol/internet protocol (TCP/IP), token ring, IEEE 802.11a/b/g/n/x, etc. Communication networks may include, without limitation, a direct interconnection, local area network (LAN), wide area network (WAN), wireless network (e.g., using Wireless Application Protocol), the Internet, etc. Using a network interface and communication network, a device may communicate with an external device or server. These devices may include, without limitation, personal computer(s), server(s), fax machines, printers, scanners, various mobile devices such as cellular telephones, smartphones (e.g., Apple iPhone, Blackberry, Android-based phones, etc.), tablet computers, eBook readers (Amazon Kindle, Nook, etc.), laptop computers, notebooks, gaming consoles (Microsoft Xbox, Nintendo DS, Sony PlayStation, etc.), or the like. In some embodiments, the device may itself embody one or more of these devices.

In some embodiments, processor(s) may be disposed in communication with one or more memory devices (e.g., RAM 113, ROM 114, etc.) via a storage interface. The storage interface may connect to memory devices including, without limitation, memory drives, removable disc drives, etc., employing connection protocols such as serial advanced technology attachment (SATA), integrated drive electronics (IDE), IEEE-1394, universal serial bus (USB), fiber channel, small computer systems interface (SCSI), etc. The memory devices may further include a drum, magnetic disc drive, magneto-optical drive, optical drive, redundant array of independent discs (RAID), solid-state memory devices, flash devices, solid-state drives, etc.

The memory devices may store a collection of program or database components, including, without limitation, an operating system, user interface application, user/application data, etc. The operating system may facilitate resource management and operation of the device. Examples of operating systems include, without limitation, Apple Macintosh OS X, Unix, Unix-like system distributions (e.g., Berkeley Software Distribution (BSD), FreeBSD, NetBSD, OpenBSD, etc.), Linux distributions (e.g., Red Hat, Ubuntu, Kubuntu, etc.), IBM OS/2, Microsoft Windows (XP, Vista/7/8, etc.), Apple iOS, Google Android, Blackberry OS, or the like. The user interface may facilitate display, execution, interaction, manipulation, or operation of program components through textual or graphical facilities. For example, user interfaces may provide computer interaction interface elements on a display system operatively connected to a device, such as cursors, icons, check boxes, menus, scrollers, windows, widgets, etc. Graphical user interfaces (GUIs) may be employed, including, without limitation, Apple Macintosh operating systems' Aqua, IBM OS/2, Microsoft Windows (e.g., Aero, Metro, etc.), Unix X-Windows, web interface libraries (e.g., ActiveX, Java, Javascript, AJAX, HTML, Adobe Flash, etc.), or the like.

In some embodiments, a device of the present disclosure is not, or is independent of, an automated separation device, e.g., an apheresis device. Apheresis generally refers to automated blood collection device that uses a centrifugal or filtration separation to automatically withdraw whole blood from a donor, separate the whole blood into blood components, collects certain of the components (e.g., platelets), and return to the donor the remainder of the whole blood and/or remaining uncollected blood components. Plateletpheresis is the collection of platelets using such an automated blood cell separator device, which results in obtaining a high yield of platelets (e.g., apheresis platelets) from a single donor. Some automated blood cell separator devices are capable of collection procedures not only for single platelet units, but also double and triple platelet units. Apheresis collection devices are well known in the art, with several such devices commercially available, including for example, the Amicus™ system (Fenwal, Inc), the Trima Accel® system (Terumo BCT) and the MCS®+ 9000 mobile system (Haemonetics, Inc).

In some embodiments, a device of the present disclosure is not, or is independent of, a blood collection device.

In some embodiments, a device of the present disclosure is not, or is independent of, an illumination device, e.g., a device configured to illuminate a blood composition in admixture with a photoactive pathogen inactivation compound (e.g., for a duration and at an intensity sufficient to inactivate a pathogen in the blood composition when present). For example, in some embodiments, an illumination device comprises a treatment chamber configured to receive a biological fluid (e.g., a blood composition); one or more sensors configured to detect light in the treatment chamber; and a first array of light sources positioned to illuminate the biological fluid in the treatment chamber, wherein the first array of light sources comprises a first light source channel configured to emit ultraviolet light (e.g., ultraviolet light with a first peak wavelength of the first array of from about 315 nm to about 350 nm), and wherein the first light source channel comprises one or more light sources, each of which emits light having a full-width half-maximum (FWHM) spectral bandwidth of less than 20 nanometers. In some embodiments, the systems further comprise a first platform positioned in the treatment chamber, the first platform configured to carry the biological fluid. Such illumination devices can be configured to illuminate the biological fluid in admixture with a photoactive pathogen inactivation compound for a duration and at an intensity sufficient to inactivate a pathogen in the biological fluid when present, e.g., configured to illuminate the biological fluid in admixture with a photoactive pathogen inactivation compound for a duration and at an intensity sufficient to inactivate at least 1 log of a pathogen in the biological fluid when present, and the biological fluid comprises 5 μM or less of photoactive pathogen inactivation compound after illuminating. Exemplary and non-limiting illumination devices are described, e.g., in International Pub. No. WO2019/133929.

It should be noted that, despite references to particular computing paradigms and software tools herein, the computer program instructions with which embodiments of the present subject matter may be implemented may correspond to any of a wide variety of programming languages, software tools and data formats, and be stored in any type of volatile or nonvolatile, non-transitory computer-readable storage medium or memory device, and may be executed according to a variety of computing models including, for example, a client/server model, a peer-to-peer model, on a stand-alone computing device, or according to a distributed computing model in which various of the functionalities may be effected or employed at different locations. In addition, references to particular algorithms herein are merely by way of examples. Suitable alternatives or those later developed known to those of skill in the art may be employed without departing from the scope of the subject matter in the present disclosure.

It will also be understood by those skilled in the art that changes in the form and details of the implementations described herein may be made without departing from the scope of this disclosure. In addition, although various advantages, aspects, and objects have been described with reference to various implementations, the scope of this disclosure should not be limited by reference to such advantages, aspects, and objects. Rather, the scope of this disclosure should be determined with reference to the appended claims.

Preferred embodiments are described herein. Variations of those preferred embodiments may become apparent to those working in the art upon reading the foregoing description. It is expected that skilled artisans will be able to employ such variations as appropriate, and the practice of the methods, systems and compositions described herein otherwise than as specifically described herein. Accordingly, the methods, systems and compositions described herein include all modifications and equivalents of the subject matter recited in the claims appended hereto as permitted by applicable law. Moreover, any combination of the above-described elements in all possible variations thereof is encompassed by the description unless otherwise indicated herein or otherwise clearly contradicted by context.

### List of Numbered Embodiments

Embodiment 1. A method of preparing a pathogen inactivated blood composition, comprising:

at a device comprising one or more components configured to control flow of one or more fluids:
(a) coupling a container containing a pathogen inactivation compound (PIC container) to a first fluid path, optionally wherein the first fluid path is loaded to the device prior to coupling the PIC container to the first fluid path;
(b) coupling a processing set to the first fluid path;
(c) transferring a blood composition from a container containing the blood composition (blood composition container) into a container of the processing set configured for mixing the blood composition with the PIC (mixing container);
(d) determining an amount of a pathogen inactivation compound (PIC) to pathogen-inactivate the blood composition based at least in part on one or more input parameters; and
(e) transferring the determined amount of the PIC from the PIC container into the mixing container via the first fluid path using the one or more components configured to control flow of fluids.

Embodiment 2. The method of embodiment 1, wherein the first fluid path is configured for multiple transfers of PIC.

Embodiment 3. The method of embodiment 1 or embodiment 2, further comprising, prior to (a): loading the first fluid path to the device.

Embodiment 4. The method of any one of embodiments 1-3, wherein the mixing container is further configured for subjecting the blood composition in admixture with the PIC to ultraviolet light.

Embodiment 5. The method of any one of embodiments 1-4, wherein the amount of the PIC to pathogen-inactivate the blood composition is determined based at least in part on weight and/or volume of the blood composition.

Embodiment 6. The method of embodiment 5, wherein the amount of the PIC to pathogen-inactivate the blood composition is determined based at least in part by calculating the volume of the blood composition based on the weight of the blood composition.

Embodiment 7. The method of any one of embodiments 1-6, wherein determining the amount of the PIC to pathogen-inactivate the blood composition further comprises weighing the blood composition in the blood composition container.

Embodiment 8. The method of embodiment 7, wherein the device further comprises a scale, and the blood composition is weighed in the blood composition container using the scale to determine the weight of the blood composition; wherein optionally the scale provides for motion of the blood composition.

Embodiment 9. The method of any one of embodiments 1-8, wherein the device further comprises one or more components configured to receive an input, and determining the amount of the PIC to pathogen-inactivate the blood composition further comprises receiving an input indicating the weight of the blood composition.

Embodiment 10. The method of embodiment 9, wherein the input further indicates the determined amount of the PIC.

Embodiment 11. The method of embodiment 9 or embodiment 10, wherein the one or more components configured to receive an input comprise a scanner, optionally wherein the scanner is a barcode scanner, radio frequency identification (RFID) scanner, or quick response (QR) code scanner.

Embodiment 12. The method of any one of embodiments 9-11, wherein the device further comprises a scale, and the received input comprises data representing the weight of the blood composition from the scale.

Embodiment 13. The method of any one of embodiments 9-12, wherein the one or more components configured to receive an input comprise a touchscreen display or keyboard, and the received input is a user input from the touchscreen display or keyboard.

Embodiment 14. The method of any one of embodiments 1-13, wherein the one or more input parameters comprise one or more of: a type of the blood composition, a volume of the blood composition, a number of platelets or red blood cells in the blood composition, a type of solution in the blood composition, a type of PIC, a target PIC concentration, and a weight of the blood composition.

Embodiment 15. The method of any one of embodiments 1-14, wherein the device further comprises one or more processors, and the amount of the PIC to pathogen-inactivate the blood composition is determined using the one or more processors.

Embodiment 16. The method of any one of embodiments 1-15, further comprising reconstituting the PIC in a solution.

Embodiment 17. The method of any one of embodiments 1-16, wherein the blood composition is transferred into the mixing container via a fluid path using gravity flow.

Embodiment 18. The method of any one of embodiments 1-16, wherein the one or more components configured to control flow of fluids comprise a pump.

Embodiment 19. The method of embodiment 18, wherein the determined amount of the PIC is transferred into the mixing container via the first fluid path using the pump.

Embodiment 20. The method of embodiment 18 or embodiment 19, wherein the blood composition is transferred into the mixing container via a fluid path using the pump.

Embodiment 21. The method of any one of embodiments 1-20, wherein the blood composition is transferred into the mixing container prior to coupling the mixing container to the first fluid path or after coupling the mixing container to the first fluid path.

Embodiment 22. The method of any one of embodiments 1-20, wherein the device further comprises one or more supports configured to support the PIC container and/or the blood composition container.

Embodiment 23. The method of any one of embodiments 1-22, further comprising terminating fluid transfer from the PIC container into the mixing container via the first fluid path using one or more of the components configured to control flow of fluids based at least in part on detecting transfer of the determined amount of the PIC into the mixing container.

Embodiment 24. The method of any one of embodiments 1-23, wherein the device further comprises one or more fluid detectors configured to detect fluid transfer within the first fluid path.

Embodiment 25. The method of any one of embodiments 1-24, further comprising detecting transfer of the determined amount of the PIC into the mixing container via the first fluid path using one or more fluid detectors.

Embodiment 26. The method of embodiment 25, further comprising: terminating fluid transfer from the PIC container into the mixing container via the first fluid path using one or more of the components configured to control flow of fluids based at least in part on detecting transfer of the determined amount of the PIC through the first fluid path using one or more fluid detectors.

Embodiment 27. The method of embodiment 25 or embodiment 26, wherein transfer of the determined amount of the PIC into the mixing container via the first fluid path is detected by measuring a volume of fluid transferred through the first fluid path using the one or more fluid detectors.

Embodiment 28. The method of any one of embodiments 24-27, wherein the device further comprises one or more fluid detectors configured to detect fluid transfer from the blood composition container via a fluid path into the mixing container, and the method further comprises detecting transfer of the blood composition into the mixing container via the fluid path using the one or more fluid detectors.

Embodiment 29. The method of embodiment 28, further comprising: terminating fluid transfer from the blood composition container into the mixing container via the fluid path using the one or more components configured to control flow of fluids based at least in part on detecting transfer of the blood composition through the second fluid path using the one or more fluid detectors.

Embodiment 30. The method of any one of embodiments 1-29, wherein the device further comprises a scale, and the method further comprises weighing the mixing container before, during, and/or after transfer of the determined amount of the PIC into the mixing container.

Embodiment 31. The method of embodiment 30, further comprising: terminating fluid transfer from the PIC container into the mixing container via the first fluid path using one or more of the components configured to control flow of fluids based at least in part on detecting transfer of a weight corresponding to the determined amount of the PIC into the mixing container using the scale.

Embodiment 32. The method of any one of embodiments 1-31, wherein the first fluid path comprises sterile tubing.

Embodiment 33. The method of embodiment 32, wherein the first fluid path further comprises one or more of: a connector, a breakable connector, a cannula, a filter, a manifold, a pump, a container, and a coupling.

Embodiment 34. The method of any one of embodiments 1-33, further comprising coupling a container containing a diluent (diluent container) to the first fluid path.

Embodiment 35. The method of embodiment 34, wherein transferring the determined amount of the PIC from the PIC container into the mixing container via the first fluid path comprises:
(i) transferring diluent from the diluent container into the PIC container; and
(ii) transferring an amount of the diluent solution and the PIC to provide the determined amount of the PIC from the PIC container into the mixing container.

Embodiment 36. The method of embodiment 34, wherein transferring the determined amount of the PIC from the PIC container into the mixing container via the first fluid path comprises:
(i) transferring PIC from the PIC container into the diluent container; and
(ii) transferring an amount of the diluent solution and the PIC to provide the determined amount of the PIC from the diluent container into the mixing container.

Embodiment 37. The method of embodiment 34, wherein transferring the determined amount of the PIC from the PIC container into the mixing container via the first fluid path comprises:
(i) transferring diluent from the diluent container into a third container, wherein the first fluid path further comprises the third container;
(ii) transferring PIC from the PIC container into the third container; and
(iii) transferring diluent and the determined amount of the PIC from the third container into the mixing container.

Embodiment 38. The method of embodiment 37, wherein transferring the diluent and the PIC into the third container comprises transferring the diluent and the PIC via a manifold, wherein the third container is connected to the manifold by a fluid path.

Embodiment 39. The method of any one of embodiments 35-38, wherein the PIC in the PIC container is at a concentration at least about 2 times greater than the concentration of the determined amount of PIC (e.g., diluted PIC) transferred into the mixing container (e.g., than required to provide a solution comprising the diluent and the determined amount of the PIC).

Embodiment 40. The method of embodiment 39, wherein the PIC in the PIC container is at a concentration about 2 times to about 50 times greater than the concentration of the determined amount of PIC (e.g., diluted PIC) transferred into the mixing container (e.g., than required to provide a solution comprising the diluent and the determined amount of the PIC).

Embodiment 41. The method of any one of embodiments 35-38, wherein the determined amount of the PIC is transferred into the mixing container at a volume accuracy of within about 2% of the determined amount.

Embodiment 42. The method of any one of embodiments 35-40, wherein amount of the diluent solution transferred into the PIC, the amount of PIC transferred into the diluent, or the amount of PIC and diluent transferred into the third container is transferred at a volume accuracy of within about 2% of a targeted volume.

Embodiment 43. The method of any one of embodiments 34-42, wherein the diluent comprises a saline solution or water.

Embodiment 44. The method of any one of embodiments 1-43, wherein, after the determined amount of the PIC is transferred into the mixing container via the first fluid path, less than about 0.3 mL of residual fluid is remaining in the first fluid path.

Embodiment 45. The method of any one of embodiments 1-44, further comprising, after or concurrently with (e): admixing the blood composition and the determined amount of the PIC in the mixing container.

Embodiment 46. The method of any one of embodiments 1-45, wherein the device is configured to agitate or rock the mixing container after or concurrently with transferring the determined amount of the PIC into the mixing container.

Embodiment 47. The method of embodiment 45 or embodiment 46, wherein the device further comprises a scale, and the blood composition and the determined amount of the PIC are admixed in the mixing container on the scale.

Embodiment 48. The method of any one of embodiments 1-47, wherein the device is not an automated separation device.

Embodiment 49. The method of any one of embodiments 1-47, wherein the device is independent of an automated separation device.

Embodiment 50. The method of any one of embodiments 1-49, wherein the device is independent of an illumination device.

Embodiment 51. The method of any one of embodiments 1-50, wherein the processing set comprises:
(i) the mixing container, within which the blood composition in admixture with the determined amount of the PIC can be subjected to ultraviolet light; and
(ii) at least a first storage container, coupled to the mixing container, wherein the first storage container is configured for storing the pathogen-inactivated blood composition; wherein optionally the processing set comprises one or more filters.

Embodiment 52. The method of any one of embodiments 1-51, wherein the blood composition comprises a platelet and/or a plasma composition.

Embodiment 53. The method of embodiment 52, wherein after (e) the PIC is at a concentration of about 40p M and about 70p M in the mixing container with the blood composition.

Embodiment 54. The method of embodiment 53, wherein after (e) the PIC is at a concentration of about 50p M and about 60p M, optionally about 55 μM, in the mixing container with the blood composition.

Embodiment 55. The method of any one of embodiments 52-54, wherein the one or more components configured to control flow of fluids comprise a pump, and wherein (e) comprises transferring the determined amount of the PIC from the PIC container into the mixing container via the first fluid path using the pump.

Embodiment 56. The method of any one of embodiments 52-55, wherein the mixing container is configured for subjecting the blood composition in admixture with the PIC to ultraviolet light under sterile conditions.

Embodiment 57. The method of embodiment 56, further comprising, after (e): subjecting the blood composition to illumination in the mixing container using the determined amount of the PIC.

Embodiment 58. The method of embodiment 57, further comprising, after subjecting the blood composition to illumination: transferring the pathogen-inactivated blood composition from the mixing container into one or more storage containers of the processing set.

Embodiment 59. The method of any one of embodiments 51-58, wherein the method does not comprise subjecting the pathogen-inactivated blood composition to a compound removal step, optionally wherein the method does not comprise contacting the pathogen-inactivated blood composition with a compound adsorption device (CAD).

Embodiment 60. The method of embodiment 57, further comprising, after subjecting the blood composition to illumination: transferring the pathogen-inactivated blood composition from the mixing container into a container containing a CAD of the processing set.

Embodiment 61. The method of any one of embodiments 52-60, further comprising calculating a light dose for subjecting the blood composition and PIC to ultraviolet light to photochemically inactivate pathogen(s) if present in the blood composition using the determined amount of the PIC based at least in part on one or more of: a type of the blood composition, a volume of the blood composition, a type of PIC, a concentration of the PIC, a volume of the container for illumination, a number of platelets in the blood composition, a type of solution in the blood composition, and a weight of the blood composition.

Embodiment 62. The method of embodiment 61, further comprising calculating a light dose for subjecting the blood composition and PIC to ultraviolet light to photochemically inactivate pathogen(s) if present in the blood composition using the determined amount of the PIC based at least in part on a volume of the blood composition and a concentration of the PIC, and optionally a volume of the container for illumination.

Embodiment 63. The method of embodiment 61 or embodiment 62, further comprising transferring data indicating the calculated light dose to a second device or server.

Embodiment 64. The method of embodiment 61 or embodiment 62, further comprising transferring to a second device or server data to calculate a light dose for photochemical inactivation of the blood composition using the determined amount of the PIC.

Embodiment 65. The method of embodiment 63 or embodiment 64, wherein the second device is an illumination device.

Embodiment 66. The method of any one of embodiments 52-65, wherein the PIC is a photoactive pathogen inactivating compound selected from the group consisting of a psoralen, an isoalloxazine, an alloxazine, a phthalocyanine, a phenothiazine, a porphyrin, and merocyanine 540.

Embodiment 67. The method of embodiment 66, wherein the PIC is a psoralen, optionally wherein the PIC is amotosalen.

Embodiment 68. The method of any one of embodiments 52-67, wherein the determined amount of the PIC is transferred into the mixing container at a volume accuracy of within about 2% of the determined amount.

Embodiment 69. The method of any one of embodiments 1-50, wherein the processing set comprises:
(i) the mixing container, which is configured for mixing the blood composition and the determined amount of the PIC; and
(ii) a second container, coupled to the container, wherein the second container is configured for incubating the blood composition and the determined amount of the PIC; wherein optionally the processing set comprises one or more filters (e.g., 0.2 μm filters).

Embodiment 70. The method of embodiment 69, wherein the processing set further comprises: (iii) at least a first storage container, coupled to the second container, wherein the first storage container is configured for storing the pathogen-inactivated blood composition.

Embodiment 71. The method of embodiment 69 or embodiment 70, wherein the mixing container contains a processing solution.

Embodiment 72. The method of any one of embodiments 69-71, wherein the storage container contains an additive solution.

Embodiment 73. The method of any one of embodiments 1-50 and 69-72, wherein the blood composition comprises red blood cells (RBCs).

Embodiment 74. The method of embodiment 73, further comprising mixing the blood composition with a quencher.

Embodiment 75. The method of embodiment 74, further comprising mixing the blood composition with a processing solution, optionally wherein mixing with the processing solution with the blood composition occurs prior to mixing the quencher with the blood composition.

Embodiment 76. The method of embodiment 74 or embodiment 75, wherein mixing the blood composition with a quencher comprises: at a second device comprising one or more components configured to control flow of one or more fluids:
(i) coupling a container containing a quencher (quencher container) to a first fluid path at the second device;
(ii) coupling a processing set to the first fluid path at the second device;
(iii) determining an amount of quencher to quench a pathogen inactivation compound (PIC) based at least in part on one or more input parameters; and
(iv) transferring the determined amount of the quencher from the quencher container into a container of the processing set configured for mixing the blood composition with the quencher (mixing container) using one or more of the components configured to control flow of fluids of the second device;
wherein the blood composition is transferred from the blood composition container into the mixing container in performance of step (c) before coupling the processing set of (b) to the first fluid path at the first device.

Embodiment 77. The method of embodiment 76, wherein the mixing container contains a processing solution.

Embodiment 78. The method of embodiment 76, further comprising transferring a processing solution from a processing solution container into the mixing container using one or more of the components configured to control flow of fluids at the second device.

Embodiment 79. The method of any one of embodiments 76-78, wherein the quencher is added to the mixing container before the blood composition is added to the mixing container.

Embodiment 80. The method of embodiment 78 or embodiment 79, wherein the processing solution is added to the mixing container before the blood composition is added to the mixing container.

Embodiment 81. The method of any one of embodiments 74-80, wherein the quencher and blood composition are mixed prior to mixing the PIC and blood composition.

Embodiment 82. The method of any one of embodiments 76-81, further comprising uncoupling the mixing container containing the blood composition and the quencher from the second device and coupling the uncoupled mixing container to the first device.

Embodiment 83. The method of embodiment 74 or embodiment 75, further comprising:
(i) coupling a quencher container to a second fluid path; and
(ii) transferring quencher from the quencher container into the mixing container via the second fluid path using one or more of the components configured to control flow of fluids.

Embodiment 84. The method of embodiment 83, wherein the quencher is added to the mixing container before the blood composition is added to the mixing container or after the blood composition is added to the mixing container.

Embodiment 85. The method of embodiment 83 or embodiment 84, wherein the mixing container contains a processing solution.

Embodiment 86. The method of embodiment 83 or embodiment 84, further comprising transferring a processing solution from a processing solution container into the mixing container using one or more of the components configured to control flow of fluids at the second device.

Embodiment 87. The method of any one of embodiments 83-86, wherein the quencher and blood composition are mixed prior to mixing the PIC and blood composition.

Embodiment 88. The method of embodiment 86 or embodiment 87, wherein the processing solution is added to the mixing container before the blood composition is added to the mixing container.

Embodiment 89. The method of embodiment 83, wherein the one or more components configured to control flow of fluids comprise a manifold; wherein the PIC container is connected to the manifold via a first segment of the first fluid path; wherein the manifold is connected to the mixing container via a second segment of the first fluid path, and wherein the quencher container is connected to the manifold via the second fluid path; optionally, wherein the quencher container is connected to the manifold via a first segment of the second fluid path; wherein the manifold is further connected to the mixing container via a second segment of the second fluid path.

Embodiment 90. The method of embodiment 89, wherein the second segment of the first fluid path comprises a Y-junction, and wherein the blood composition container is connected to the Y-junction.

Embodiment 91. The method of embodiment 89 or embodiment 90, wherein the manifold is operably linked to an air filter.

Embodiment 92. The method of embodiment 83, wherein the one or more components configured to control flow of fluids comprise a first manifold and a second manifold; wherein the PIC container is connected to the first manifold via a first segment of the first fluid path; wherein the manifold is connected to the processing set (e.g., mixing container) via a second segment of the first fluid path, and wherein the quencher container is connected to the second manifold via the second fluid path, optionally, wherein the quencher container is connected to the second manifold via a first segment of the second fluid path; and wherein the manifold is further connected to the processing set (e.g., mixing container) via a second segment of the second fluid path; optionally, the method further comprises coupling a processing solution container to the second fluid path and transferring the processing solution to the processing set using the second manifold.

Embodiment 93. The method of any one of embodiments 76-92, further comprising, prior to transferring the quencher from the quencher container into the mixing container: reconstituting the quencher in a liquid solution.

Embodiment 94. The method of any one of embodiments 73-93, wherein the PIC comprises a nucleic acid binding ligand that is an intercalator, optionally wherein the intercalator is an acridine.

Embodiment 95. The method of any one of embodiments 73-94, wherein the PIC is (3-alanine, N-(acridin-9-yl), 2-[bis(2-chloroethyl)amino]ethyl ester.

Embodiment 96. The method of any one of embodiments 74-95, wherein the quencher comprises cysteine or a derivative of cysteine.

Embodiment 97. The method of any one of embodiments 74-95, wherein the quencher is a peptide of 3-6 amino acids, wherein at least one of the amino acids is cysteine, N-acetyl cysteine, or S-acetyl cysteine.

Embodiment 98. The method of any one of embodiments 74-97, wherein the quencher is glutathione or a pharmaceutically acceptable salt thereof, optionally wherein the quencher is glutathione monosodium salt.

Embodiment 99. The method of any one of embodiments 70-98, wherein the determined amount of the PIC is transferred into the mixing container at a volume accuracy of within about 2% of the determined amount.

Embodiment 100. The method of any one of embodiments 74-99, wherein the quencher is transferred into the mixing container at a volume accuracy of within about 2% of the determined amount.

Embodiment 101. The method of any one of embodiments 1-100, wherein the first fluid path is configured for multiple uses.

Embodiment 102. The method of embodiment 101, wherein the first fluid path is part of a reusable consumable configured for use in a dosing device system, wherein the reusable consumable comprises one or more fluid path(s), one or more connector(s), one or more manifold(s), and optionally one or more pump(s).

Embodiment 103. The method of any one of embodiments 1-101, wherein the PIC container, the processing set, and/or the mixing container are reversibly coupled to the first fluid path.

Embodiment 104. The method of any one of embodiments 76-103, wherein the quencher container is reversibly coupled to the fluid path.

Embodiment 105. The method of any one of embodiments 103-104, wherein the reversible coupling is a sterile, reversible coupling; optionally, wherein the sterile, reversible coupling is using a sterile connector.

Embodiment 106. The method of any one of embodiments 76-105, further comprising uncoupling the processing set from the first fluid path and the second fluid path.

Embodiment 107. The method of any one of embodiments 76-105, further comprising transferring the mixture of the blood composition, PIC, quencher and processing solution into the second container and incubating the mixture.

Embodiment 108. The method of embodiment 107, further comprising replacing the PIC, quencher, and processing solution with an additive solution.

Embodiment 109. The method of embodiment 108, wherein replacing the PIC, quencher and processing solution with an additive solution, comprising connecting a storage container containing the additive solution to the second container, separating the PIC, quencher and processing solution from the RBCs, and mixing the additive solution with the RBCs to yield pathogen inactivated RBC blood composition.

Embodiment 110. A system for preparing a pathogen-inactivated blood composition, comprising:
(a) a fluid path (e.g., a first fluid path) configured to be removably mounted to a device for controlling flow of one or more fluids, wherein the fluid path is configured to be coupled to a container containing a pathogen inactivation compound (PIC), and configured to be coupled to a processing set, and wherein the fluid path is configured for transfer of the PIC to the processing set; optionally wherein the fluid path is configured to be coupled to the container containing the PIC and to the processing set via one or more connectors;
(b) a device comprising one or more components configured to control flow of one or more fluids through the fluid path; and
(c) the processing set, comprising:
   (i) a container configured for mixing a blood composition with the PIC (mixing container), wherein the mixing container is configured to be coupled to the fluid path; and at least a first storage container, coupled to the mixing container, wherein the storage container is configured for storing the pathogen-inactivated blood composition; or
   (ii) a container configured for mixing a blood composition with a PIC (mixing container), wherein the mixing container is configured to be coupled to the fluid path; a second container, coupled to the mixing container, wherein the second container is configured for incubating the blood composition in admixture with the PIC and the second container is configured to be connected to at least a first storage container configured for storing the pathogen-inactivated blood composition; or
   (iii) a container configured for mixing a blood composition with a PIC (mixing container), wherein the mixing container is configured to be coupled to the fluid path; a second container, coupled to the mixing container, wherein the second container is configured for incubating the blood composition and the PIC; and at least a first storage container, coupled to the second container, wherein the storage container is configured for storing the pathogen-inactivated blood composition; or
   (iv) a container configured for mixing a blood composition with a PIC (mixing container), wherein the mixing container is configured to be coupled to the fluid path; a second container, coupled to the mixing container, wherein the second container contains a compound adsorption device (CAD); and at least a first storage container, coupled to the second container, wherein the storage container is configured for storing the pathogen-inactivated blood composition.

Embodiment 111. The system of embodiment 110, wherein the first fluid path is configured for multiple transfers of PIC.

Embodiment 112. The system of embodiment 110 or embodiment 111, wherein the first fluid path is part of a reusable consumable of the system, wherein the reusable consumable comprises one or more fluid paths, one or more connectors, one or more manifolds, and optionally one or more pumps; optionally wherein the first fluid path comprises a manifold and/or a pump.

Embodiment 113. The system of any one of embodiments 110-112, wherein the mixing container is configured for subjecting a blood composition and PIC contained therein to ultraviolet light.

Embodiment 114. The system of any one of embodiments 110-112, further comprising a second fluid path configured to be removably mounted to a device for controlling flow of one or more fluids, wherein the second fluid path is configured to be coupled to a container containing a quencher, and configured to be coupled to the processing set, and wherein the second fluid path is configured for transfer of quencher to the processing set; optionally, wherein the second fluid path is part of a reusable consumable of the system, wherein the reusable consumable comprises one or more fluid paths, one or more connectors, one or more manifolds, and optionally one or more pumps; optionally, wherein the second fluid path comprises a manifold and/or a pump.

Embodiment 115. The system of embodiment 114, further comprising a third fluid path configured to be removably mounted to a device for controlling flow of one or more fluids, wherein the third fluid path is configured to be coupled to a container containing a processing solution, and wherein the third fluid path is connected to a manifold or configured to be coupled to the processing set, and wherein the third fluid path is configured for transfer of processing solution to the processing set.

Embodiment 116. The system of any one of embodiments 110-115, further comprising a fourth fluid path configured to be removably mounted to a device for controlling flow of one or more fluids, wherein the fourth fluid path is configured to be coupled to a container containing a diluent solution, and wherein the fourth fluid path is connected to a manifold, and wherein the manifold is configured for mixing the PIC and the diluent.

Embodiment 117. The system of embodiment 116, wherein the manifold is configured for mixing the PIC and the diluent in a container (dilution container) connected to the manifold by a fluid path.

Embodiment 118. The system of any one of embodiments 110-117, wherein the device further comprises one or more components configured to receive an input; optionally, wherein the input comprises one or more of a type of the blood composition, a volume of the blood composition, a number of platelets or red blood cells in the blood composition, a type of solution in the blood composition, a type of PIC, a target concentration of PIC, and a weight of the blood composition.

Embodiment 119. The system of embodiment 118, wherein the one or more components configured to receive an input comprise one or more of a scanner, barcode scanner, radio frequency identification (RFID) scanner, quick response (QR) code scanner, touchscreen display, or keyboard.

Embodiment 120. The system of any one of embodiments 110-119, wherein the device further comprises a scale configured to weigh a blood composition and/or the mixing container.

Embodiment 121. The system of any one of embodiments 110-120, wherein the device further comprises one or more processors configured to determine an amount of the PIC to add to the blood composition to pathogen-inactivate the blood composition; optionally, wherein the amount of the PIC is determined based at least in part on weight and/or volume of the blood composition; optionally, wherein the amount of the PIC is determined based at least in part by calculating the volume of the blood composition based on the weight of the blood composition.

Embodiment 122. The system of any one of embodiments 114-121, wherein the device further comprises one or more processors configured to determine an amount of the quencher to add to the blood composition to pathogen inactivate the blood composition; optionally, wherein the amount of the quencher is determined based at least in part on weight and/or volume of the blood composition; optionally, wherein the amount of the quencher is determined based at least in part by calculating the volume of the blood composition based on the weight of the blood composition.

Embodiment 123. The system of any one of embodiments 110-113 and 116-121, wherein the device is configured to calculate a light dose for subjecting the blood composition and PIC to ultraviolet light to photochemically inactivate pathogen(s) if present in the blood composition using the PIC based at least in part on one or more of: a type of the blood composition, a volume of the blood composition, a type of PIC, a concentration of the PIC, a volume of the container for illumination, a number of platelets in the blood composition, a type of solution in the blood composition, and a weight of the blood composition.

Embodiment 124. The system of embodiment 123, wherein the device is configured to calculate a light dose for subjecting the blood composition and PIC to ultraviolet light using the PIC based at least in part on a volume of the blood composition and a concentration of the PIC, and optionally a volume of the container for illumination.

Embodiment 125. The system of embodiment 123 or embodiment 124, wherein the device is configured to transfer data indicating the calculated light dose to a second device or server. Embodiment 126. The system of embodiment 123 or embodiment 124, wherein the device is configured to transfer to a second device or server data to calculate a light dose for subjecting the blood composition and PIC to ultraviolet light to photochemically inactivate pathogen(s) if present in the blood composition using the PIC.

Embodiment 127. The system of embodiment 125 or embodiment 126, wherein the second device is an illumination device.

Embodiment 128. The system of any one of embodiments 110-127, wherein the one or more components configured to control flow of fluids comprise a pump and/or manifold.

Embodiment 129. The system of any one of embodiments 110-128, wherein the device further comprises one or more supports configured to support the PIC container and/or the blood composition container.

Embodiment 130. The system of any one of embodiments 110-129, wherein the device further comprises one or more fluid detectors configured to detect fluid transfer from the blood composition container into the mixing container.

Embodiment 131. The system of any one of embodiments 110-130, wherein the system is configured to terminate fluid transfer from the PIC container into the mixing container via the first fluid path using one or more of the components configured to control flow of fluids based at least in part on detecting transfer of the determined amount of the PIC into the mixing container; optionally, based at least in part on detecting transfer of the determined amount of the PIC through the first fluid path using the one or more fluid detectors.

Embodiment 132. The system of any one of embodiments 110-131, wherein the system and/or the fluid path comprises one or more of: a connector, a breakable connector, a cannula, a filter, a manifold, a pump, a container, and a coupling.

Embodiment 133. The system of any one of embodiments 110-132, wherein the fluid path further comprises one or more of: a connector, a breakable connector, a cannula, a filter, a manifold, a pump, a container, and a coupling.

Embodiment 134. The system of any one of embodiments 110-133, wherein the device comprises a scale, and the system is configured to weigh the mixing container before, during, and/or after transfer of the determined amount of the PIC into the mixing container; optionally, wherein the system is configured to terminate fluid transfer from the PIC container into the mixing container via the using one or more of the components configured to control flow of fluids based at least in part on detecting transfer of a weight corresponding to the determined amount of the PIC into the mixing container using the scale.

Embodiment 135. The system of any one of embodiments 110-134, wherein the system is configured to transfer a determined amount of PIC to a processing set at a volume accuracy of within about 2% of the determined amount.

Embodiment 136. The system of any one of embodiments 110-135, wherein the system is configured to transfer and mix an amount of diluent solution and an amount of PIC, wherein the amount of PIC is transferred into the diluent, of the amount of diluent is transferred into the PIC, or the amount of PIC and the amount of diluent are transferred into a container, wherein the amount is transferred at a volume accuracy of within about 2% of a targeted volume.

Embodiment 137. The system of any one of embodiments 110-136, wherein the system is configured to transfer a determined amount of quencher to a processing set at a volume accuracy of within about 2% of the determined amount.

Embodiment 138. The system of any one of embodiments 110-137, wherein the system is configured to calculate a light dose for subjecting the blood composition and PIC to ultraviolet light to photochemically inactivate pathogen(s) if present in a blood composition using a determined amount of the PIC based at least in part on one or more of: a type of the blood composition, a volume of the blood composition, a type of PIC, a concentration of the PIC, a volume of the container for illumination, a number of platelets in the blood composition, a type of solution in the blood composition, and a weight of the blood composition.

Embodiment 139. The system of embodiment 138, wherein the system is configured to calculate a light dose for subjecting the blood composition and PIC to ultraviolet light to photochemically inactivate pathogen(s) if present in a blood composition using a determined amount of the PIC based at least in part on a volume of the blood composition and a concentration of the PIC, and optionally a volume of the container for illumination.

Embodiment 140. The system of any one of embodiments 110-139, wherein the device is not an automated separation device.

Embodiment 141. The system of any one of embodiments 110-139, wherein the device is independent of an automated separation device.

Embodiment 142. The system of any one of embodiments 110-141, wherein the device is independent of an illumination device.

Embodiment 143. The system of any one of embodiments 110-142, wherein the processing set is configured to be reversibly coupled to one or more fluid paths, optionally, wherein the processing set comprises a fluid path configured for coupling (e.g., via connector(s)) to a first fluid path from a PIC container and/or a second fluid path from a quencher container; and/or wherein the processing set comprises a fluid path configured to be connected to a blood composition container; and/or wherein the processing set comprises one or more filters.

Embodiment 144. The system of any one of embodiments 110-143, wherein the fluid path(s) is/are configured to be mounted on the device.

Embodiment 145. The system of any one of embodiments 110-144, wherein the system is configured to alert a system operator when a multi-dose bulk container of solution should be replaced.

Embodiment 146. The system of any one of embodiments 110-145, wherein the system is configured to alert a system operator when a reusable consumable portion of the system should be replaced.

The invention is illustrated further by the following examples, which are not to be construed as limiting the invention in scope or spirit to the specific procedures described in them.

### Examples

### Example 1: UVA Light Dose and Unit Volume Relationship in Treating Platelet Units

Pathogen inactivation systems for blood components targeting nucleic acids of pathogens, such as the psoralen-based INTERCEPT™ Blood Systems for platelets and plasma and the amustaline-based INTERCEPT™ Blood System for red blood cells (Cerus Corporation), currently use processing sets that deliver a single, fixed volume of pathogen inactivation compound (e.g., psoralen, amustaline) into a varying volume range of blood components (e.g. within defined “guard bands”), thus resulting in varying concentrations of pathogen inactivation compound in the blood component mixture. Because of the varied blood component volumes, the added pathogen inactivation compound is more concentrated in smaller blood component volumes and less concentrated in larger blood component volumes within the permissible range, which may lead to potentially decreased pathogen inactivation levels at lower concentrations and/or decreased health of treated blood components and/or higher residual pathogen inactivation compound (e.g., above acceptable levels) after inactivation at higher concentration.

In the case of psoralen-based pathogen inactivation, excess residual psoralen is removed to below acceptable levels (e.g., levels approved by regulatory authorities) using a compound adsorption device (CAD) as part of the processing set. The CAD component adds greater manufacturing cost and processing time to the pathogen inactivation systems.

The studies disclosed herein evaluated whether such pathogen inactivation systems could be improved by switching to dosing with a fixed concentration of pathogen inactivation compound (e.g., psoralen) across a range of varied volumes of blood component along with switching to varied UVA light doses that correspond to the varied blood component volumes for photochemical inactivation rather than subjecting the varied volumes of blood component/psoralen mixture to a fixed dose of ultraviolet A (UVA) light to photochemically inactivate pathogen(s) that may be present in the blood component.

Multiple studies were performed to determine the level of residual compound remaining after photochemical treatment of platelets with a psoralen for pathogen inactivation. More specifically, the studies were performed using a range of platelet volumes (e.g., 200 mL-450 mL; 50 mL volume increments) and using a single initial input concentration of the psoralen compound, amotosalen (S-59) by changing input volume. For each volume tested, units of platelets, prepared in 47% plasma/53% PAS, were pooled and split into two to three individual units, each in an illumination container of an INTERCEPT® Blood System processing set. The individual units were each dosed with amotosalen from an initial starting concentration (e.g., stock solution) at a volume needed to achieve a final 70 μM target concentration for each platelet volume, mixed well, and subjected to illumination with UVA light. The doses of UVA light tested for each volume included, for example, one or more light doses ranging from 2.5 J/cm² to 6.5 J/cm². (e.g., 2.5-4.5 J/cm² for 200 mL, 3.5-4.0 J/cm² for 250 mL 4.5-6.5 J/cm² for 300 mL, 5.2 J/cm² for 350 mL, 5.5-6.5 J/cm² for 400 mL, 6.4-6.5 J/cm² for 450 mL). UVA treatment was performed in the illumination container using an illuminator device with LED light (e.g., narrow bandwidth) having peak wavelength between 340-350 nm. Samples were collected both pre- and post-UVA illumination for HPLC analysis of amotosalen concentration.

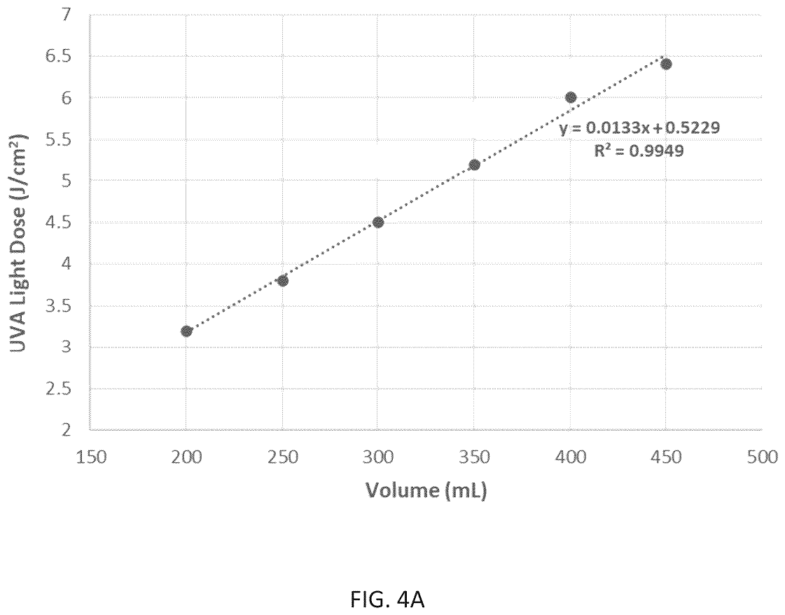

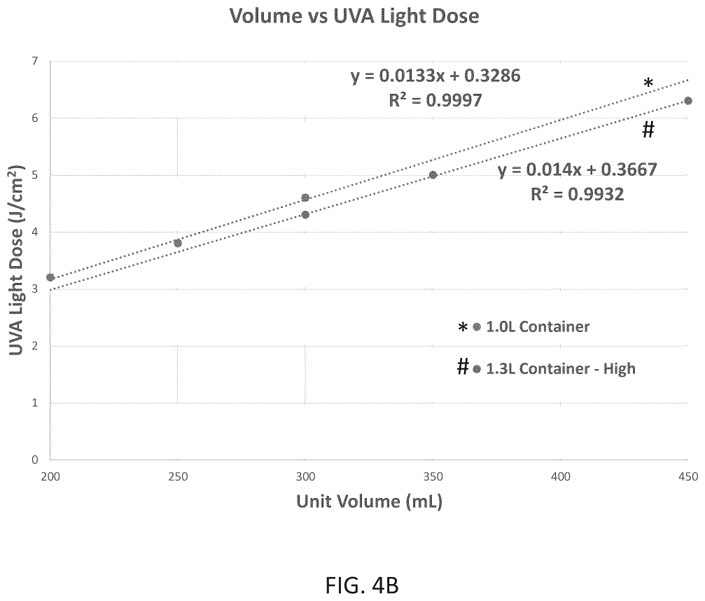

From the multiple studies above, UVA light doses that provided low residual amotosalen levels (e.g., less than 7.5 μM) post-UVA treatment were selected for each specified platelet volume. These light doses are shown in Table 1 below, together with a corresponding measured volume of platelets, which the light dose may be used in conjunction with. This exemplary light dose and volume relationship identified in the studies may be further defined in the graph of FIG. 4A, which plots UVA light dose versus volume for achieving low residual amotosalen levels.

These studies demonstrated that quantized dosing of amotosalen at a specified target concentration for each platelet volume to be treated provided an opportunity for improving the amotosalen/UVA pathogen inactivation process (e.g., decreased levels of residual amotosalen post-UVA). As the dosing methods in these studies were manual addition of amotosalen, variability was observed in both input and residual amotosalen concentrations (Table 1), indicating that the amotosalen/UVA process may be further improved with greater accuracy in dosing of the compound.

TABLE 1
Residual amotosalen (S-59) levels in treated platelets.

| Volume | UVA | Pre-UVA | Post-UVA |
|---|---|---|---|
| (mL) | (J/cm²) | S-59 [μM] | S-59 [μM] |
| 200 | 3.2 | 71.1 ± 2.4 | 4.5 ± 1.5 |
| 250 | 3.8 | 71.5 ± 3.7 | 5.0 ± 1.1 |
| 300 | 4.5 | 72.5 ± 3.2 | 5.1 ± 1.3 |
| 350 | 5.2 | 74.8 ± 2.0 | 5.0 ± 0.6 |
| 400 | 6.0 | 69.8 ± 2.6 | 4.4 ± 0.8 |
| 450 | 6.4 | 68.5 ± 0.8 | 5.2 ± 0.8 |

### Example 2: Inactivation of Klebsiella pneumoniae

A study was performed to evaluate pathogen inactivation with treatment profiles from Example 1 and the bacterium Klebsiella pneumoniae. Units of platelets, prepared in 47% plasma/53% PAS at a volume of 250 mL (except the 70 μM study group at a volume of 200 mL), were inoculated with an overnight culture of Klebsiella pneumoniae (targeting the final concentration in unit of approximately 6 log CFU/mL) and dosed with the target amotosalen concentration (40 μM, 45 μM, 55 μM or 70 μM) in an illumination container of an INTERCEPT® Blood System processing set. The units were mixed well, and samples were collected from each unit prior to UV illumination to determine pre-treatment bacterial titers and amotosalen concentration. The Klebsiella-contaminated platelet units were then subjected to a UVA light dose of 3.8 J/cm² (3.2 J/cm² for the 200 mL units) using an illuminator with LED light sources having peak wavelength between 340-350 nm. Samples were collected post-illumination and both the pre- and post-UVA illumination samples were analyzed by standard colony forming unit assay to determine the level of bacterial inactivation. As shown in Table 2 below, bacterial inactivation (log reduction) of greater than 5 logs was obtained with each of the four target amotosalen input concentrations tested.

TABLE 2
Bacterial inactivation with amotosalen/UVA

| Target S-59 | Pre-UVA | Post-UVA | |
|---|---|---|---|
| concentration | Log CFU/mL | CFU/mL | Log Reduction |
| 40 μM | 6.0 ± 0.0 | 6.7 ± 11.5 | 5.6 ± 0.8 |
| 45 μM | 5.9 ± 0.0 | 0.2 ± 0.1 | 5.9 ± 0.0 |
| 55 μM | 6.0 ± 0.1 | 0.4 ± 0.5 | 6.0 ± 0.1 |
| 70 μM | 6.1 ± 0.1 | 0.03 ± 0.1 | ≥6.1 ± 0.1 |

### Example 3: Inactivation of Bluetongue Virus

A study was performed to evaluate pathogen inactivation with treatment profiles from Example 1 and the non-enveloped virus bluetongue virus (BTV). Units of platelets, prepared in 47% plasma/53% PAS at a volume of 250 mL, were inoculated with BTV (targeting the final concentration in unit of approximately 4.5 log PFU/mL), and dosed with the target amotosalen concentration (40 μM, 50 μM, 60 μM, or 70 μM) in an illumination container of an INTERCEPT® Blood System processing set. The units were mixed well, and samples were collected from each unit prior to UV illumination to determine pre-treatment viral titers and amotosalen concentration. The BTV-contaminated platelet units were then subjected to a UVA light dose of 3.8 J/cm² using an illuminator with LED light sources having peak wavelength between 340-350 nm. Samples were collected post-illumination, and both the pre- and post-UVA illumination samples were analyzed by standard plaque assay to determine the level of virus inactivation. As shown in Table 3 below, viral inactivation greater than 4 logs was obtained with each of the four target amotosalen input concentrations tested.

TABLE 3
Viral inactivation with amotosalen/UVA

| Target S-59 | Pre-UVA | Post-UVA | |
|---|---|---|---|
| concentration | Log PFU/mL | PFU/mL | Log Reduction |
| 40 μM | 4.4 ± 0.1 | 0.3 ± 0.1 | 4.4 ± 0.1 |
| 50 μM | 4.4 ± 0.4 | 0.1 ± 0.1 | 4.4 ± 0.4 |
| 60 μM | 4.6 ± 0.5 | 0.02 ± 0.03 | >4.6 ± 0.5 |
| 70 μM | 4.2 ± 0.1 | 0.1 ± 0.2 | 4.2 ± 0.1 |

### Example 4: Inactivation of Adenovirus Type 5

Another study was performed evaluating pathogen inactivation with treatment profiles from Example 1 and the non-enveloped virus adenovirus type 5 (Ad5). Units of platelets, prepared in 47% plasma/53% PAS at a volume of 250 mL, were inoculated with Ad5 (targeting the final concentration in unit of approximately 4.5 or 6.5 log PFU/mL), and dosed with the target amotosalen concentration (45 μM or 50 μM) in an illumination container of an INTERCEPT® Blood System processing set. The units were mixed well, and samples were collected from each unit prior to UV illumination to determine pre-treatment viral titers and amotosalen concentration. The Ad5-contaminated platelet units were then subjected to a UVA light dose of 3.8 J/cm² using an illuminator with LED light sources having peak wavelength between 340-350 nm. Samples were collected post-illumination and both the pre- and post-UVA illumination samples were analyzed by standard plaque assay to determine the level of virus inactivation. As shown in Table 4 below, viral inactivation greater than 4 logs was obtained with each of the 45 μM or 50 μM target amotosalen input concentrations.

TABLE 4
Viral inactivation with amotosalen/UVA

| Target S-59 | Pre-UVA | Post-UVA | |
|---|---|---|---|
| concentration | Log PFU/mL | PFU/mL | Log Reduction |
| 50 μM | 6.7 ± 0.3 | ≤6.9 ± 12.7 | ≥6.3 ± 0.5 |
| 50 μM | 4.5 ± 0.0 | 0.0 ± 0.0 | 4.5 ± 0.0 |
| 45 μM | 4.1 ± 0.2 | 0.0 ± 0.0 | >4.1 ± 0.2 |

### Example 5: Residual Amotosalen Levels in Treated Platelet Units

Residual amotosalen was also determined from the above pathogen inactivation studies and collectively summarized in Table 5 below. Each of the 40 μM, 50 μM, 60 μM and 70 μM test conditions used 250 mL sample volumes and UVA light doses of 3.8 J/cm². Amotosalen concentrations were determined before and after illumination with UVA. The data show that for each of the tested amotosalen concentrations, residual amotosalen was below 7.5 μM, and also <5.0 μM.

TABLE 5
Residual amotosalen concentration pre- and post-UVA

| Target S-59 | | |
|---|---|---|
| concentration | Pre-UVA [μM] | Post-UVA [μM] |
| 40 μM | 40.8 ± 1.2 | 2.3 ± 0.7 |
| 50 μM | 50.7 ± 3.3 | 3.0 ± 0.8 |
| 60 μM | 62.0 ± 0.3 | 3.9 ± 1.4 |
| 70 μM | 71.5 ± 3.2 | 5.0 ± 1.1 |

### Example 6: Dosing Device System-Controlled Fluid Delivery

To evaluate if variability in dosing observed in Example 1 that resulted from manual addition of solution containing the pathogen inactivation compound could be addressed, further studies including development and testing of a dosing device system for controlled fluid transfer. The computer processor-controlled dosing device comprised the following components, among others: a peristaltic pump through which tubing can be mounted (e.g., routed), pinch valves through which tubing can be mounted, container supports and a scale.

Fluid is transferred from a container of pathogen inactivation compound (PIC) and from a container of biological fluid to be treated into a mixing container (e.g., illumination container) by way of sterile tubing, with the tubing passing through device-controlled pinch valves as a means to open and close the flow of fluid. The flow of biological fluid from its container positioned on an upper support of the device is by gravity into the mixing container, which is positioned on the scale in a lower portion of the device. The flow of PIC from its container also positioned on an upper support of the device additionally passes through the peristaltic pump to control delivery of a determined amount of PIC into the mixing container.

The scale in the dosing device, in conjunction with computer processor, provides at least two functions: 1) determining the weight of the biological fluid to be treated (e.g., after the fluid is flowed from its initial container into the mixing container), and 2) determining when a determined amount of PIC has been added to the mixing container (e.g., added to the biological fluid). From the weight of the biological fluid to be treated, the device can determine a corresponding volume of the biological fluid, and an amount of PIC (e.g., suitable amount) to be added to the volume of biological fluid, such as for example, to achieve a desired level of pathogen inactivation and/or reduce the level of residual PIC below a certain threshold.

The dosing device was tested for dosing accuracy, initially using a solution of water+dye to represent the PIC (e.g., with similar density) and using water only to represent the biological fluid. Dosing for these studies was performed using a new processing set configuration. This processing set comprises a mixing container (e.g., illumination container) coupled by way of a first port of the container to a fluid path (e.g., sterile tubing) for transfer of the biological fluid and PIC into the container (e.g., a receiving fluid path). The tubing comprises a junction (e.g., Y-junction) from which two fluid paths (e.g., sterile tubing) extend, one which is coupled (or configured to be coupled) to a container containing the PIC and the other which is configured to be coupled to a container containing the biological fluid, thereby allowing for the flow of PIC and biological fluid from their respective containers to be individually controlled. The mixing container in some embodiments may be coupled by way of a second port of the container to another fluid path (e.g., output fluid path), which is further coupled (or configured to be coupled) to a storage container and/or a CAD container.

For testing, the biological fluid (water substitute) was used at unit target volumes of either 200 mL or 450 mL, as shown below for two studies (Tables 6A and 6B). Each test unit was transferred into the mixing container, and based on the unit volume, a target amount of PIC (water+dye substitute) for delivery was determined, as shown in Tables 6a and 6b. PIC delivery was controlled by the device using weight determination and the actual amount of PIC delivered is also shown below. PIC amounts are stated in grams (g) and correspond to a volume (mL) using known density of an S-59 solution (1.001060 g/mL). These data demonstrate that the dosing device achieved high levels of accuracy within 1% in all except one case, which was 1.2%.

TABLE 6A
Accuracy of device-controlled delivery.

| Targeted | PIC quantity | PIC quantity | PIC Accuracy |
|---|---|---|---|
| volume (mL) | targeted (g) | delivered (g) | (volume) (%) |
| 200 | 9.8 | 9.77 | 0.3 |
| 200 | 9.68 | 9.68 | 0 |
| 200 | 9.12 | 9.08 | 0.4 |
| 200 | 8.99 | 8.94 | 0.6 |
| 200 | 9.76 | 9.73 | 0.3 |
| 200 | 10.23 | 10.22 | 0.1 |
| 200 | 9.39 | 9.34 | 0.5 |
| 200 | 9.18 | 9.21 | 0.3 |
| 200 | 9.45 | 9.4 | 0.5 |
| 200 | 8.31 | 8.38 | 0.8 |
| 450 | 21.12 | 21.28 | 0.8 |
| 450 | 21.54 | 21.52 | 0.1 |
| 450 | 22.35 | 22.35 | 0 |

TABLE 6B
Accuracy of device-controlled delivery.

| Targeted | PIC quantity | PIC quantity | PIC Accuracy |
|---|---|---|---|
| volume (mL) | targeted (g) | delivered (g) | (volume) (%) |
| 200 | 9.12 | 9.17 | 0.5 |
| 200 | 9.01 | 8.97 | 0.4 |
| 200 | 9.72 | 9.69 | 0.3 |
| 200 | 8.3 | 8.3 | 0 |
| 200 | 9.44 | 9.49 | 0.5 |
| 200 | 9.69 | 9.64 | 0.5 |
| 200 | 9.47 | 9.44 | 0.3 |
| 200 | 9.47 | 9.44 | 0.3 |
| 200 | 9.38 | 9.49 | 1.2 |
| 200 | 9.62 | 9.59 | 0.3 |
| 450 | 21.38 | 21.4 | 0.1 |
| 450 | 20.41 | 20.38 | 0.1 |
| 450 | 21.67 | 21.66 | 0 |

Additionally, controlled dosing of pathogen inactivation compound into a volume of biological fluid with a high degree of accuracy, together with a defined with a corresponding UVA light dose determined for any specific volume within a desired range of volumes (see e.g., Example 1), provides an opportunity for further improved pathogen inactivation systems.

From the above Examples, additional studies evaluated the device-controlled dosing of pathogen inactivation compound while varying treatment volumes in either of two illumination container sizes. More specifically, a first study was performed to demonstrate the accuracy of dosing of a psoralen pathogen inactivation compound into platelet additive solution (PAS). Amotosalen (S-59) from two different stock concentrations 1.05 mM and 1.54 mM was dosed with the dosing device into four replicates of 200 mL or 300 mL of PAS in a 1.0 L illumination container and 300 mL or 450 mL of PAS in a 1.3 L illumination container, respectively, with a target final S-59 concentration of 55 μM. After dosing, S-59 concentration was determined by HPLC with the results shown in the following Table 7. These data confirm the high level of dosing accuracy for pathogen inactivation compound (e.g., S-59 solution) delivered by a device of the present disclosure.

TABLE 7
Accuracy of device-controlled delivery of compound

| | |
|---|---|
| 1.0 L Container, | 1.3 L Container, |
| 1.05 mM S-59 | 1.54 mM S-59 |

| Rep # | 200 mL | 300 mL | 300 mL | 450 mL |
|---|---|---|---|---|
| 1 | 54.28 | 55.10 | 54.45 | 55.43 |
| 2 | 56.67 | 54.45 | 54.07 | 54.32 |
| 3 | 55.40 | 54.08 | 53.37 | 55.57 |
| 4 | 53.96 | 54.99 | 54.54 | 53.79 |
| Average | 55.08 | 54.66 | 54.11 | 54.78 |
| SD | 1.23 | 0.48 | 0.53 | 0.86 |

After confirming the accuracy of S-59 dosing into PAS, the next study evaluated dosing of S-59 into a blood component, platelets suspended in plasma and PAS, and photoconversion of S-59 post-UVA treatment to determine residual S-59 levels and generate (e.g., plot) a volume to light dose curve, similar to Example 1. Different size illumination containers (1.0 L and 1.3 L) were evaluated with multiple different platelet volumes and multiple different UVA light doses used for photochemical treatment of each volume and container size.

More specifically, platelets suspended in 43% plasma/53% PAS at volumes of 200 mL, 250 mL or 300 mL in a 1.0 L illumination container, and 300 mL, 350 mL or 450 mL in a 1.3 L illumination container, were dosed at varying input volumes of the S-59 psoralen compound to achieve a target final concentration of 55 μM. Samples were taken to confirm pre-illumination S-59 concentration and each of the S-59 containing platelet volumes was then illuminated with different light doses as shown in the following Tables 8A and 8B using an illuminator device with LED light sources (e.g., narrow bandwidth) having peak wavelength between 340-350 nm. The concentration of S-59 pre- and post-illumination was then analyzed by HPLC to confirm dosing accuracy and to determine light doses sufficient to achieve photoconversion at each volume that resulted in residual S-59 concentration below a desired target threshold, such as for example <7.5 μM or a concentration (e.g., 3-4 μM) for which three standard deviations from a mean remains <7.5 μM. These results demonstrate an advantage of the present invention, in that pathogen-inactivated platelets may be prepared with no further psoralen removal step, such as for example using a compound adsorption device (CAD), required after illumination to achieve the low residual psoralen levels (e.g., <7.5 μM) suitable for human use.

TABLE 8A
Photoconversion: 1.0 L illumination container.

| Light Dose | | 200 mL | 250 mL | 300 mL |
|---|---|---|---|---|
| 2.8 J/cm² | Pre-UVA | 53.21 ± 0.68 μM | | |
| | Post-UVA | 5.22 ± 0.50 μM | | |
| 3.0 J/cm² | Pre-UVA | 53.31 ± 0.60 μM | | |
| | Post-UVA | 4.40 ± 0.64 μM | | |
| 3.2 J/cm² | Pre-UVA | 56.27 ± 0.39 μM | | |
| | Post-UVA | 3.71 ± 0.07 μM | | |
| 3.4 J/cm² | Pre-UVA | 57.14 ± 0.24 μM | 54.72 ± 0.87 μM | |
| | Post-UVA | 3.09 ± 0.18 μM | 4.25 ± 0.13 μM | |
| 3.6 J/cm² | Pre-UVA | | 55.83 ± 0.76 μM | |
| | Post-UVA | | 3.83 ± 0.74 μM | |
| 3.8 J/cm² | Pre-UVA | | 55.27 ± 1.00 μM | |
| | Post-UVA | | 3.81 ± 0.49 μM | |
| 4.0 J/cm² | Pre-UVA | | 55.50 ± 0.72 μM | |
| | Post-UVA | | 3.11 ± 1.18 μM | |
| 4.1 J/cm² | Pre-UVA | | | 54.16 ± 0.52 μM |
| | Post-UVA | | | 7.13 ± 2.95 μM |
| 4.3 J/cm² | Pre-UVA | | | 55.89 ± 2.96 μM |
| | Post-UVA | | | 6.85 ± 3.00 μM |
| 4.6 J/cm² | Pre-UVA | | | 57.35 ± 0.70 μM |
| | Post-UVA | | | 2.86 ± 1.36 μM |
| 4.8 J/cm² | Pre-UVA | | | 57.00 ± 0.30 μM |
| | Post-UVA | | | 3.63 ± 1.66 μM |

TABLE 8A
Photoconversion: 1.3 L illumination container.

| Light Dose | | 300 mL | 350 mL | 450 mL |
|---|---|---|---|---|
| 4.1 J/cm² | Pre-UVA | 55.43 ± 2.18 μM | | |
| | Post-UVA | 4.64 ± 0.13 μM | | |
| 4.3 J/cm² | Pre-UVA | 56.94 ± 1.42 μM | | |
| | Post-UVA | 3.43 ± 1.30 μM | | |
| 4.8 J/cm² | Pre-UVA | | 55.33 ± 1.77 μM | |
| | Post-UVA | | 3.54 ± 1.01 μM | |
| 5.0 J/cm² | Pre-UVA | | 55.96 ± 1.01 μM | |
| | Post-UVA | | 2.98 ± 0.91 μM | |
| 6.1 J/cm² | Pre-UVA | | | 53.21 ± 0.72 μM |
| | Post-UVA | | | 3.79 ± 0.49 μM |
| 6.3 J/cm² | Pre-UVA | | | 53.85 ± 0.35 μM |
| | Post-UVA | | | 3.56 ± 0.37 μM |

From these data, light doses were selected and linear regression calculated for each of the 1.0 L and 1.3 L container volume data to generate (e.g., plot) volume to UVA light dose relationship curves. The two curves for 1.0 L vs 1.3 L container size are shown in FIG. 4B, and were found to have very similar slopes (and similar to Example 1), offset in light dose which arises from the different container volumes and thus fluid depth (e.g., light transmissivity) in the container. These results demonstrate the versatility of the methods described herein based on volume to light dose relationship for determining a suitable light dose useful for pathogen inactivation of any desired volume along the curve.

### Inactivation of Bacteria and Viruses

Pathogen inactivation of both viruses and bacteria was also tested using dosing device system-based quantized dosing of amotosalen and the treatment parameters described above. In one study, four units of platelets in 47% plasma/53% PAS at a higher 450 mL volume were inoculated in a 1.3 L illumination container with bluetongue virus (BTV) at a target pathogen input concentration of approximately 5.0 log PFU/mL per unit and dosed with amotosalen (S-59) to achieve at a target concentration of 55 μM. The units were mixed, and samples were collected from each unit prior to UV illumination to determine pre-treatment viral titers and S-59 concentration. The BTV-contaminated platelet units were then subjected to a UVA light dose of 6.3 J/cm² using an illuminator with LED light sources having peak wavelength between 340-350 nm. Samples were collected post-illumination, and both the pre- and post-UVA illumination samples were analyzed by standard plaque assay to determine the level of virus inactivation and by HPLC to determine the S-59 concentration. As shown in Table 9 below, viral inactivation was 5.0±0.4 logs and residual S-59 was 3.0±0.7.

A similar study was performed with the bacterium Klebsiella pneumoniae. Four units of platelets in 47% plasma/53% PAS at a volume of 450 mL were inoculated in a 1.3 L illumination container with an overnight culture of Klebsiella pneumoniae at a target input pathogen concentration of approximately 8 log CFU/mL per unit and dosed with S-59 to achieve a target concentration of 55 μM. Samples were collected pre- and post-UVA illumination with a light dose of 6.3 J/cm² for determination of bacterial titers by standard colony forming unit assay and S-59 concentration by HPLC. As shown in Table 9 below, bacterial inactivation (log reduction) was greater than 7 logs and residual S-59 was 3.2±0.5 μM.

TABLE 9
Bacterial and viral inactivation with device-delivery S-59 psoralen.
Bluetongue virus

| Pre-UVA titer | Post-UVA titer | Log reduction | Pre-UVA | Post-UVA |
|---|---|---|---|---|
| Log (pfu/mL) | (pfu/mL) | Log (pfu/mL) | S-59 (μM) | S-59 (μM) |
| 5.0 ± 0.3 | 0.7 ± 0.6 | 5.0 ± 0.4 | 56.5 ± 0.6 | 3.0 ± 0.7 |

K. pneumoniae

| Pre-UVA titer | Post-UVA titer | Log reduction | Pre-UVA | Post-UVA |
|---|---|---|---|---|
| Log (cfu/mL) | (cfu/mL) | Log (cfu/mL) | S-59 (μM) | S-59 (μM) |
| 7.9 ± 0.1 | 0.3 ± 0.4 | 7.6 ± 0.4 | 56.0 ± 0.6 | 3.2 ± 0.5 |

Additional studies were performed to confirm the quality of platelets after pathogen inactivation with amotosalen (target concentration 55 μM) and UVA light from an illumination device with LED light sources. Platelets in PAS (35% plasma/65% PAS) were treated with S-59/UVA or untreated as control, and evaluated over a 7-day period, with common quality assessments using standard assays known in the art, such as pH, blood gases (pO2, pCO2), LDH release, extracellular glucose and acetate, platelet count and mean platelet volume, ATP, platelet activation markers (e.g., P-selectin, αIIbβ3) and phosphatidylserine expression (e.g., annexin V binding). Very similar metabolic, activation and apoptosis signal profiles were observed between the S-59/UVA-treated and untreated controls.

The multiple Examples provided above describe and demonstrate how a UV light dose may be determined for photochemical pathogen inactivation of any blood component (e.g., platelet component) volume and pathogen inactivation compound (e.g., psoralen) concentration, using the multiple exemplary illustrated blood product volumes, compound concentrations and illumination container sizes. Similarly, UV light doses may be determined to include other parameter variables, such as for example, platelet resuspension media (e.g., plasma, plasma/PAS concentrations), illumination container materials, RBC contaminant levels in platelets or plasma, etc. Such light dose determination for the UV illumination step may be calculated by the dosing device and communicated to an illumination device as an output from the dosing device (e.g., wired, wireless, labelling on platelet component). Alternatively, or in addition, such light dose determination may be calculated by the illumination device or a separate (e.g., intermediate) computing device using information provided (e.g., as output from the dosing device), such as the blood component volume and pathogen inactivation compound concentration, and any other desired parameters, such as noted above.

These studies further demonstrate that quantized dosing of psoralen-based pathogen inactivation compounds at a specific target concentration while varying platelet volumes and UV light doses improves the photochemical pathogen inactivation process (e.g., lower residual psoralen, increased inactivation of pathogen(s)), while maintaining platelet quality compared to current systems that use variable compound concentrations and fixed light doses, among other benefits such as the opportunity for automation and/or higher throughput. These results further demonstrate an advantage of the present invention, in that pathogen-inactivated platelets may be prepared with no further compound (e.g., psoralen, S-59) removal step, such as for example using a compound adsorption device (CAD), as currently required after illumination to achieve the low residual psoralen levels (e.g., <7.5 μM) for human use.

### Multi-Dose Delivery of Pathogen Inactivation Compound

Bulk dosing of pathogen inactivation compound for the treatment processing of multiple blood component donations may be performed using a dosing device system of the present disclosure. In one non-limiting example for pathogen inactivation of a blood component, precise dosing of a psoralen pathogen inactivation compound (PIC) from a multi-dose bulk container of the psoralen compound amotosalen (S-59) to multiple platelet processing sets sequentially is performed using a system comprising a device and associated “consumables” (e.g., reusable components) and processing sets of the present disclosure, wherein the system comprises a pump for use in transfer of PIC and a scale for weight based determination of a volume of platelets (e.g., in the processing set). The reusable consumable component(s) of the system comprises one or more fluid paths (e.g., tubing) and connectors and optionally one or more pumps and/or manifolds, and is contemplated to be used for the dosing of multiple processing sets (e.g., 2 or more, 12 or more, 24 or more, 48 or more, 100 or more, 200 or more) and/or for certain periods of time (e.g., 8 hours or less, 12 hours or less, 24 hours or less, operator work day) before being replaced on the device (see e.g., FIG. 8-13). The device may be configured to alert the user when the reusable consumable portion of the system should be replaced (e.g., based on elapsed time since loading the consumable portion, based on number of dosing cycles with the consumable portion). In some embodiments, the device of the system may comprise one or more pumps. In some embodiments, the consumable component may further comprise an additional fluid path for reversibly coupling a container of diluent (e.g., saline) and providing a fluid path from the diluent container to a manifold in common (e.g., shared) with a fluid path for the PIC, in order to dilute a more concentrated solution of PIC to a less concentrated PIC before transferring the PIC to the processing set and mixing with the blood component (see e.g., FIGS. 11-13). Such dilution of PIC may, in some embodiments, take place in an additional container of the consumable portion of the system, such as for example a container for dilution (dilution container) connected by a fluid path to the manifold.

After loading (e.g., mounting, installing) the consumable component of the system to the device component of the system, a multi-dose “bulk” container containing the PIC, which in some embodiments may be previously reconstituted from dry (e.g., lyophilized) form, is coupled (e.g., connected, reversibly coupled), for example with a connector, to a fluid path of the consumable component. Multi-dose “bulk” containers with diluent for dilution of the PIC is similarly coupled as needed or desired (e.g., based on PIC solution). In some embodiments, the device may be configured to alert the user when a multi-dose bulk container (e.g., PIC, diluent) should be replaced, such as for example based on elapsed time since coupling the container to the system and/or based on remaining volume in the container.

After loading (e.g., mounting, installing) the consumable component of the system to the device component of the system, and in some embodiments after coupling a bulk container of PIC to the system, a disposable processing set is coupled (e.g., connected, reversibly coupled), for example with a connector, to a fluid path of the consumable component. Any processing set comprising one or more containers configured for use in pathogen inactivation may be used, such as processing sets and related methods described in the present application. The processing set may comprise, for example, container(s) configured for mixing (e.g., mixing container) the blood composition and PIC, configured for subjecting the blood composition and PIC to ultraviolet light (e.g., illumination, for photochemical inactivation of pathogen(s) if present), configured for incubating the blood composition and PIC (e.g., incubation container), configured for removing residual PIC, such as for example with a compound adsorption device (CAD), and/or configured for storage of the pathogen inactivated blood composition (e.g., storage container). The blood composition (e.g., blood component) to be treated is transferred from a blood component container to the processing set (e.g., mixing container of processing set) either before or after coupling the processing set to a fluid path of the consumable portion of the system, and the blood component container is subsequently disconnected from the processing set after transfer, such as for example by heat sealing of connecting tubing. Transferring the blood composition to the processing set prior to coupling the processing set to the consumable portion of the system may provide certain advantages such as higher throughput PIC dosing of multiple processing sets sequentially as the blood composition to be treated is already in the processing set before coupling.

In some embodiments, the volume of the blood composition (e.g., in the mixing container) is determined (e.g., by the system), such as for example by first determining the weight of the blood composition and calculating a volume therefrom using conversion factors known in the art. The weight may be obtained using a scale (e.g., subtracting the tare weight of the mixing container), which scale may in some embodiments be a component of the device (e.g., internal or external to a device housing). The device (e.g., processor(s) of the device) determines an amount of PIC to pathogen-inactivate the blood composition based at least in part on one or more parameters, such as for example the type of blood composition (e.g., platelets, plasma, RBCs, whole blood, cryoprecipitate, cryo-reduced plasma), weight and/or volume of the blood composition, a type of solution in the blood composition (e.g., additive solution, buffer, plasma), and/or a number of platelets or RBCs in the blood composition.

For one non-limiting example of photochemical pathogen inactivation of platelets with a psoralen (e.g., amotosalen), the device may obtain the weight of a platelet composition suspended in 47% plasma/53% PAS in a mixing container of a processing set (e.g., the mixing container also serving as an illumination container), and calculate the volume of the platelet composition to be 350 mL. Additional information about the platelet composition may, in some embodiments, be obtained with a device scanner. In this example, a desired final PIC concentration target of 55 μM for pathogen inactivation is selected and input to the device (e.g., user touchscreen display) in the 350 mL platelet composition volume and a known concentration of amotosalen solution in a multi-dose bulk container, the device determines the amount of PIC to transfer from the PIC container to the processing set (e.g., mixing container of processing set containing the platelets). The determined amount of PIC is then transferred from the PIC container to the mixing container of the processing set (e.g., while rocking the mixing container) by way of a fluid path of the disposable consumable component with flow of fluid controlled as described in the present application to ensure accuracy of PIC dosing within about 2% of the determined amount (e.g., 2% or better). The device further calculates a light dose for subjecting the platelets and PIC to ultraviolet A light (e.g., with peak wavelength between 330-350 nm) to photochemically inactivate pathogen(s) that may be present in the platelet composition. The light dose is calculated, for example, based at least in part on the platelet composition volume, the type of solution in the platelet composition (e.g., plasma and PAS), the PIC concentration, the illumination container volume (e.g., 1.3 L) and the previously determined volume-light dose relationship (volume-light dose curve, described above). Data indicating the calculated dose (e.g., 4.8 J/cm², to achieve residual amotosalen levels <7.5 μM, inclusive of 3 standard deviations) is then communicated as an output from the dosing device to an illumination device for subjecting the platelets and PIC to ultraviolet light, such as for example by wired (e.g., network) connection or by printing a label to affix to the processing set containing the platelets and PIC. Alternatively, such light dose determination may be calculated by the illumination device or a separate (e.g., intermediate) computing device using information provided by the dosing device (e.g., as output data from the dosing device). The illumination device is then used to complete the photochemical inactivation step.

### Example 7: Improved Stability of Amustaline (S-303)

Pathogen inactivation of RBCs uses the pathogen inactivation compound amustaline dihydrochloride (amustaline, S-303) and the quencher glutathione (GSH). Single fixed volumes of S-303 and GSH are used to treat a range of 220 mL-360 mL volumes of RBCs in additive solution in the single use INTERCEPT® Blood System disposable processing set. Following reconstitution in saline, approximately 15 mL of 600 mM GSH is added to the mixing bag of the processing set, which contains 140 mL of processing solution, followed by the addition of RBCs to the mixing bag within 4 hours, or as little as 1 hour depending on country specific approvals. S-303 is then also reconstituted in saline at a concentration of 6 mM and 15 mL is added to the mixing bag with RBC+GSH within 10 minutes of S-303 reconstitution due to instability of the S-303 compound (e.g., related to its labile ester bond) and a requirement of less than 5% degradation prior to use.

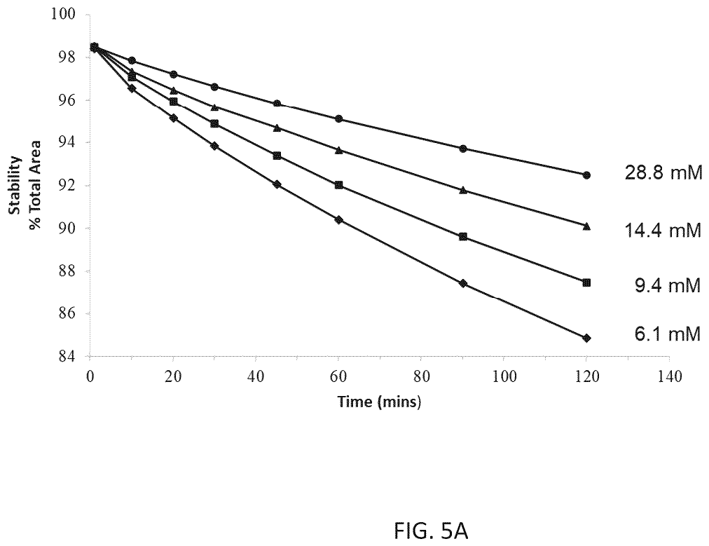

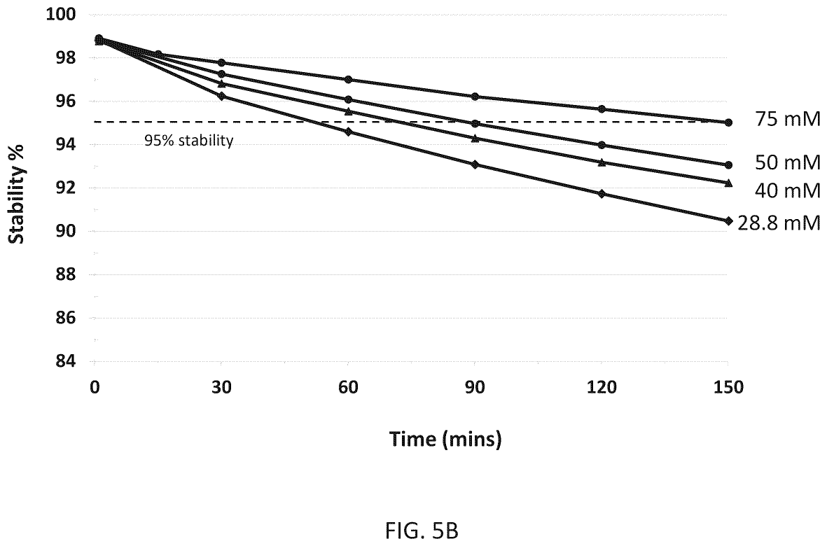

Increasing the throughput of RBC processing over the current process may benefit from the preparation of multi-use “bulk” containers of S-303 and GSH stock solutions for transfer (e.g., sequential transfer) into multiple processing sets to treat multiple RBC units. However, the instability of S-303 after reconstitution in the current INTERCEPT Blood System technology would generally not be suitable for such a multi-dose container approach. Toward an objective of multi-dose “bulk” dosing, which would require much longer hold times post-reconstitution, studies were performed to evaluate the stability of S-303 under different conditions, such as when reconstituted at higher concentrations. As control, S-303 was reconstituted at a concentration of 6.1 mM. Additional study arms reconstituted S-303 at 9.4 mM, 14.4 mM and 28.8 mM (Table 10). S-303 levels were measured over 120 min after reconstitution by standard HPLC and calculated as total peak area (e.g., area under the peak, area under the curve) to determine percent stability (percent total area). As shown in Table 10 and FIG. 5A, S-303 was surprisingly found to have increased stability at higher concentration and was >95% stable up to 60 minutes when reconstituted at 28.8 mM.

TABLE 10
Stability of S-303 at different concentrations.

| | Control | | | |
|---|---|---|---|---|
| | 6.1 mM | 9.4 mM | 14.4 mM | 28.8 mM |
| Time | Mean % | Mean % | Mean % | Mean % |
| (min) | stability | stability | stability | stability |
| 1 | 98 | 98 | 99 | 99 |
| 10 | 97 | 97 | 97 | 98 |
| 20 | 95 | 96 | 96 | 97 |
| 30 | 94 | 95 | 96 | 97 |
| 45 | 92 | 93 | 95 | 96 |
| 60 | 90 | 92 | 94 | 95 |
| 90 | 87 | 90 | 92 | 94 |
| 120 | 85 | 87 | 90 | 92 |

Subsequent studies were performed in a similar manner as the above to further evaluate S-303 stability at higher concentrations than tested initially, including reconstitution at 40 mM, 50 mM and 75 mM concentrations. As shown in FIG. 5B, S-303 exhibited even greater stability over time at higher concentrations, including stability of at least 95% through 150 min following reconstitution at 75 mM concentration, which would be sufficient for a multi-dose bulk container approach. Additionally, for the 75 mM concentration, stability was compared between S-303 in glass vials as used for the current INTERCEPT™ Blood System and S-303 in hemocompatible blood bags of different sheeting materials, with no differences in stability observed. Extrapolation of the data in a best fit curve further indicates that even higher concentrations of S-303 should continue to increase stability, such as for example approximately 170 min at 100 mM concentration and 240 min at 150 mM concentration.

The extended stability for S-303 achieved by reconstituting at higher concentrations, together with the precision dosing device system-based dosing approach for pathogen inactivation compound demonstrated in Examples 1-6, provides an opportunity to use multi-dose bulk containers and a dosing device (e.g., as described in the present application) for higher throughput dosing (e.g., sequential dosing) into multiple RBC processing sets, such as for example, bulk containers of compound being configured for dosing (directly or with dilution prior to dosing) at least 24 processing sets, which greatly exceeds the throughput capacity of the existing INTERCEPT™ Blood System.

### Example 8: Reconstitution in Alternative Solutions

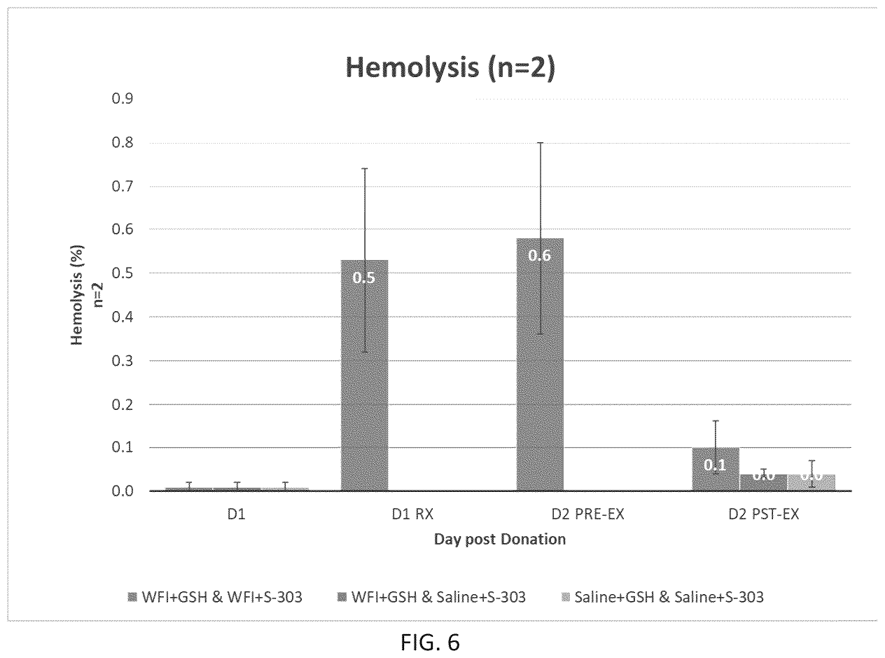

A study was performed to evaluate water (WFI, water for injection) as an alternative to saline for reconstitution of S-303 and/or GSH, for potential advantages such as manufacturing, cost of goods and/or regulatory requirements. Initially, the combinations of S-303 and GSH both reconstituted in WFI, or alternatively GSH reconstituted in WFI and S-303 reconstituted in saline were compared to the standard method of reconstituting both GSH and S-303 in saline. Following addition to RBCs, the level of hemolysis was evaluated due to water potentially being a hypotonic environment for the RBC. As shown in FIG. 6, reconstitution of S-303 in particular, resulted in notable levels of hemolysis not observed with saline reconstitution.

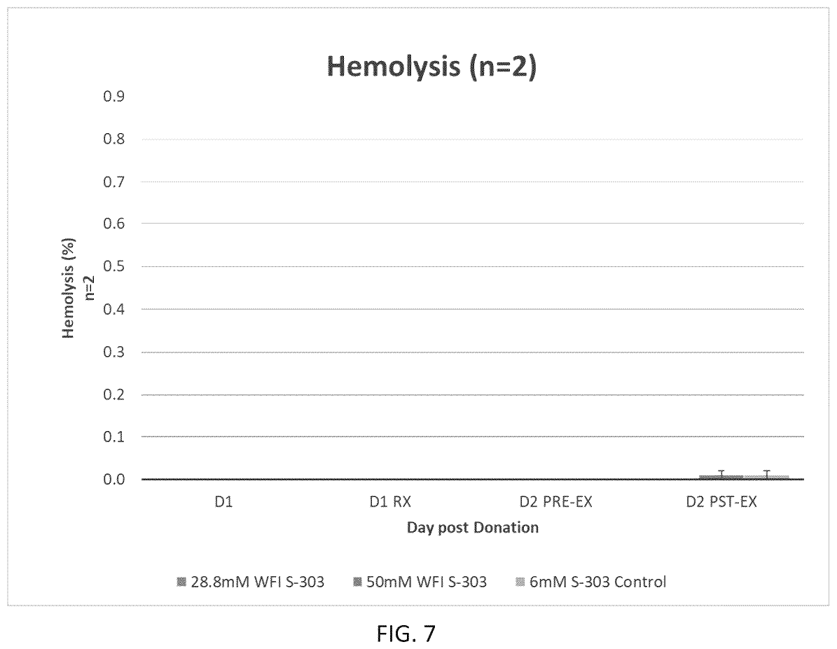

Based on the extended stability data above for reconstituting S-303 at higher concentrations, an opportunity is provided for using bulk S-303 containers and a dosing device as described above for reconstituting S-303 at higher concentrations followed by a dilution step with diluent as part of the transfer of S-303 to the RBC in the mixing container. With such a dilution step, water may be substituted as an alternative to saline in the reconstitution step for the bulk S-303, followed by dilution in saline prior to each transfer step into individual RBC processing sets. To evaluate the feasibility of water reconstitution followed by saline dilution, an additional study was performed. In the test arms, S-303 was reconstituted at 28.8 mM or 50 mM in WFI, then diluted to 6 mM in saline prior to addition to RBCs. GSH was reconstituted in WFI in both test arms. For control, both S-303 and GSH were reconstituted in saline as standard practice. As shown in FIG. 7, reconstitution of S-303 in WFI, even at concentrations up to 50 mM, followed by dilution in saline prior to addition to RBCs, eliminated the issue of hydrolysis previously observed and verified the dilution method. A similar dilution method may be used directly with reconstitution of S-303 in saline at high concentrations in bulk containers of S-303.

### Example 9: Bulk Dosing of Amustaline (S-303) and Quencher

Reconstitution of S-303 at high concentration and the resulting extended stability provides an opportunity for using multi-dose bulk S-303 containers and a dosing device system as described in the present application for delivery of S-303 to multiple RBC processing sets sequentially rather than reconstituting S-303 individually for each disposable processing set. In addition to the S-303 pathogen inactivation compound, a quencher (e.g., GSH) may also be supplied in a multi-dose bulk container. For example, in one embodiment, a bulk container of GSH quencher is reconstituted in saline or water (e.g., in a dual chamber bag or dual chamber syringe) in a volume sufficient for dosing 6 or more, 12 or more, 18 or more, or 24 or more RBC units in disposable processing sets by reversibly coupling (e.g., connecting, sterilely coupling or connecting) the GSH container to a fluid pathway (e.g., reusable fluid path, sterile tubing) at the dosing device system, coupling a first processing set to a fluid path (e.g., sterile tubing) at the dosing device system (e.g., by way of a connector), transferring a desired amount (e.g., 15 mL) of the reconstituted GSH with accuracy of at least 2% into the mixing container of the processing set, which mixing container already contains processing solution and admixing, and transferring the RBCs into the mixing container and admixing. A bulk container of S-303 is reconstituted in saline or water (e.g., in a dual chamber bag or syringe), such as for example at 50 mM, in a sufficient volume to dose 6 or more, 12 or more, 18 or more, or 24 or more RBC units in the disposable processing sets by reversibly (and sterilely) coupling the S-303 container to a fluid path (e.g., sterile tubing) at the dosing device system (e.g., by way of a connector), a portion of the S-303 (e.g., 2.22 mL) sufficient for dosing the unit of RBCs is diluted to 6 mM with saline, from a bulk saline container also connected to the dosing device system, at an accuracy within 2%, and a desired volume (e.g., 18.5 mL) of the diluted S-303 is then transferred with an accuracy of at least 2% to the mixing container of the processing set that contains processing solution into which quencher and the RBCs have already been added, uncoupling (e.g., disconnecting) the processing set for incubation and further processing, and repeating with the next processing set, up to any maximum number of processing sets for the bulk containers of GSH and S-303. Containers with any of the bulk solutions may be disconnected from the dosing device system and replaced with new containers of bulk solution(s) when a container is empty or as otherwise determined, such as for example, by the device operator or an alert from the device (e.g., if a permissible time since reconstitution and/or connecting to the device has expired). In some embodiments, rather than a dosing the same fixed volume of PIC and quencher for a range of RBC volumes, the dosing device system-controlled dosing of PIC and/or quencher comprises transferring a specific amount that is determined based on the volume of the actual RBC unit to be treated. In some embodiments, the volume may be determined by weighing the RBC unit and the dosing device further comprises a scale for weighing the unit. Based on the determined weight, the device calculates the RBC unit volume and a required dose of PIC and/or quencher for that volume to transfer into the mixing container.

Bulk dosing of PIC and quencher from multi-dose containers to multiple RBC processing sets may also be performed using system comprising a device and associated “consumables” (e.g., reusable components) of the present disclosure, wherein the system comprises a pump and a manifold (see e.g. FIG. 14-17) for use in transfer of PIC and quencher. Such configurations may provide for transfer of PIC and quencher by way of separately controlled fluid paths for accurate dosing and maintaining separation of the PIC and quencher until at or a short distance prior to (e.g., “Y-junction” portion of processing set) the fluid being transferred into a mixing container of the processing set. The reusable consumable component(s) of the system comprises one or more fluid paths, connectors, manifolds and in certain embodiments pumps, and is contemplated to be used for the dosing of multiple processing sets (e.g., sequentially, 2 or more, 12 or more, 24 or more, 48 or more, 100 or more, 200 or more) and/or for certain periods of time (e.g., 8 hours or less, 12 hours or less, 24 hours or less, operator work day) before being replaced on the device. The device may be configured to alert the user when the reusable consumable portion of the system should be replaced (e.g., based on elapsed time since loading the consumable portion, based on number of dosing cycles with the consumable portion, based on incomplete, improper, or inaccurate dosing). In some embodiments, the device of the system may comprise one or more pumps and/or controllers and/or actuators of pumps. In some embodiments, the consumable component may further comprise separate “output” fluid paths from the manifold to maintain separation of PIC and quencher (e.g., through the device associated consumable) see e.g., FIG. 16-17. In some embodiments, the consumable component may further comprise an additional fluid path for reversibly coupling a container of diluent (e.g., saline) and providing a fluid path from the diluent container to a manifold in common (e.g., shared) with a fluid path for the PIC, in order to dilute a more concentrated solution of PIC to a less concentrated PIC before transferring the PIC to the processing set and mixing with the blood component (e.g., RBCs) see e.g., FIG. 14-17. Such dilution of PIC may, in some embodiments, take place in an additional container of the consumable portion of the system, such as for example a container for dilution (dilution container) connected by a fluid path to the manifold. In some embodiments, the consumable component may further comprise an additional fluid path for reversibly coupling a container of processing solution and providing a fluid path from the processing solution container to a manifold in common (e.g., shared) with a fluid path for the quencher, for transferring the processing solution to the processing set (e.g., for processing set configurations that do not already contain processing solution) and mixing with the quencher, such as for example before mixing the quencher and blood component (e.g., RBCs) see e.g., FIG. 15, 17.

After loading (e.g., mounting, installing) the consumable component of the system to the device, a multi-dose “bulk” container containing the PIC, which may be previously reconstituted from dry (e.g., lyophilized) form, is reversibly coupled (e.g., connected) to a first fluid path of the consumable component, while a multi-dose “bulk” container containing the quencher, which may be reconstituted from a dry form, is reversibly coupled (e.g., connected) to a second fluid path of the consumable component. Multi-dose “bulk” containers containing diluent for dilution of the PIC and processing solution are similarly coupled as needed or desired (e.g., based on processing set configuration). In some embodiments, the device may be configured to alert the user when a multi-dose bulk container (e.g., PIC, quencher, processing solution, additive solution) should be replaced, such as for example based on elapsed time since coupling the container to the system and/or based on remaining volume in the container.

After loading (e.g., mounting) the consumable component of the system to the device (e.g., and after coupling the multi-dose bulk container(s)), a disposable processing set as described in the present application is reversibly coupled (e.g., connected) to a fluid path of the consumable component. The blood component (e.g., RBCs) to be treated is transferred from a blood component container to the processing set (e.g., mixing container of processing set) before or after coupling the processing set to a fluid path of the consumable portion of the system and the blood component container is then disconnected from the processing set, such as for example by heat sealing of connecting tubing. Transferring the blood component to the processing set prior to coupling the processing set to the consumable portion of the system may provide certain advantages such as higher throughput PIC and quencher dosing of multiple processing sets sequentially as the blood component is to be treated is already in the processing set before coupling. In some embodiments, the volume of the RBC (e.g., in the mixing container) is determined, such as for example by determining the weight of the RBC and calculating a volume therefrom, with the weight determined using a scale which may in some embodiments be a component of the device (e.g., internal or external to a device housing). An amount of PIC and/or quencher for treating the RBC may be determined from the weight and/or volume as described in the present application.

Any processing set comprising one or more containers configured for use in pathogen inactivation may be used, such as processing sets and related methods described in the present application. The processing set may comprise, for example, container(s) configured for mixing (e.g., mixing container) the blood component, PIC and quencher, configured for incubating the blood component, PIC and quencher (e.g., incubation container), and/or configured for replacing the PIC and quencher with additive solution and/or storage of the pathogen inactivated blood product (e.g., storage container). In some embodiments, the mixing container may already contain (e.g., manufactured with) a processing solution (see e.g., FIG. 14, 16). In some embodiments, the mixing container does not container processing solution and is configured for processing solution from a bulk container of processing solution to be added, such as described above (see e.g., FIG. 15, 17). In some embodiments, the disposable processing set comprises a container containing additive solution for replacing the PIC and quencher and subsequent storage of the pathogen inactivated RBCs. Such processing sets may comprise an openable seal (e.g., clamp, frangible seal) to restrict (e.g., prevent) fluid transfer between a container used for incubation of the blood component mixed with PIC (and quencher) and the additive solution container until desired by the user. In some embodiments, the container containing additive solution for replacing the PIC (and quencher) and subsequent storage of the pathogen inactivated RBCs is separate from (e.g., not part of) the processing set and may be used in conjunction with the processing set, such as for example by sterile “welding” to the processing set after incubation of the RBCs with PIC and quencher.

Multiple Pumps and/or Manifolds

Bulk dosing of PIC and quencher from multi-dose containers to multiple RBC processing sets may also be performed using system comprising a device and associated “consumables” (e.g., reusable components) of the present disclosure, wherein the system comprises two separate pumps and either a single manifold (see e.g. FIG. 18) or two separate manifolds (see e.g., FIGS. 19-21) for use in transfer of PIC and quencher. Such configurations provide for transfer of PIC and quencher by way of separately controlled fluid paths for accurate dosing and maintaining separation of the PIC and quencher until at or a short distance prior to (e.g., “y-junction” portion of processing set) the fluid being transferred into a mixing container of the processing set. The reusable consumable component(s) of the system comprises one or more fluid paths, connectors, manifolds and in certain embodiments pumps, and is contemplated to be used for the dosing of multiple processing sets (e.g., sequentially, 2 or more, 12 or more, 24 or more, 48 or more, 100 or more, 200 or more) and/or for certain periods of time (e.g., 8 hours or less, 12 hours or less, 24 hours or less, operator work day) before being replaced on the device. The device may be configured to alert the user when the reusable consumable portion of the system should be replaced (e.g., based on elapsed time since loading the consumable portion, based on number of dosing cycles with the consumable portion, based on incomplete, improper, or inaccurate dosing). In some embodiments, the device of the system may comprise one or more pumps. In some embodiments, the consumable component may further comprise an additional fluid path for reversibly coupling a container of diluent (e.g., saline) and providing a fluid path from the diluent container to a manifold in common (e.g., shared) with a fluid path for the PIC, in order to dilute a more concentrated solution of PIC to a less concentrated PIC before transferring the PIC to the processing set and mixing with the blood component (e.g., RBCs) see e.g., FIGS. 18-21. Such dilution of PIC may, in some embodiments, take place in an additional container of the consumable portion of the system, such as for example a container for dilution connected by a fluid path to the manifold. In some embodiments, the consumable component may further comprise an additional fluid path for reversibly coupling a container of processing solution and providing a fluid path from the processing solution container to a manifold in common (e.g., shared) with a fluid path for the quencher, for transferring the processing solution to the processing set (e.g., for processing set configurations that do not already contain processing solution) and mixing with the quencher, such as for example before mixing the quencher and blood component (e.g., RBCs) see e.g., FIGS. 19-21. In some embodiments, the system may further comprise an additional pump (and optionally an additional manifold) and an additional fluid path (e.g., a part of the consumable component) for reversibly coupling a container of additive solution and providing for the transfer of an additive solution from an additive solution container to the processing set, such as for example, when using processing set configurations that do not already contain additive solution (see e.g., FIG. 20).

After loading (e.g., mounting, installing) the consumable component of the system to the device, a multi-dose “bulk” container containing the PIC, which may be previously reconstituted from dry (e.g., lyophilized) form, is reversibly coupled (e.g., connected) to a first fluid path of the consumable component, while a multi-dose “bulk” container containing the quencher, which may be reconstituted from a dry form, is reversibly coupled (e.g., connected) to a second fluid path of the consumable component. Multi-dose “bulk” containers containing diluent for dilution of the PIC, processing solution and/or additive solution are similarly coupled as needed or desired (e.g., based on processing set configuration). In some embodiments, the device may be configured to alert the user when a multi-dose bulk container (e.g., PIC, quencher, processing solution, additive solution) should be replaced, such as for example based on elapsed time since coupling the container to the system and/or based on remaining volume in the container.

After loading (e.g., mounting) the consumable component of the system to the device (e.g., and after coupling the multi-dose bulk container(s)), a disposable processing set as described in the present application is reversibly coupled (e.g., connected) to a fluid path of the consumable component. The blood component (e.g., RBCs) to be treated is transferred from a blood component container to the processing set (e.g., mixing container of processing set) before or after coupling the processing set to a fluid path of the consumable portion of the system and the blood component container is then disconnected from the processing set, such as for example by heat sealing of connecting tubing. Transferring the blood component to the processing set prior to coupling the processing set to the consumable portion of the system may provide certain advantages such as higher throughput PIC and quencher dosing of multiple processing sets sequentially as the blood component is to be treated is already in the processing set before coupling. In some embodiments, the volume of the RBC (e.g., in the mixing container) is determined, such as for example by determining the weight of the RBC and calculating a volume therefrom, with the weight determined using a scale which may in some embodiments be a component of the device (e.g., internal or external to a device housing).

Any processing set comprising one or more containers configured for use in pathogen inactivation may be used, such as processing sets and related methods described in the present application. The processing set may comprise, for example, container(s) configured for mixing (e.g., mixing container) the blood component, PIC and quencher, configured for incubating the blood component, PIC and quencher (e.g., incubation container), and/or configured for replacing the PIC and quencher with additive solution and/or storage of the pathogen inactivated blood product (e.g., storage container). In some embodiments, the mixing container may already contain (e.g., manufactured with) a processing solution (see e.g., FIG. 18). In some embodiments, the mixing container does not container processing solution and is configured for processing solution from a bulk container of processing solution to be added, such as described above (see e.g., FIGS. 19-21). In some embodiments, the disposable processing set comprises a container containing additive solution for replacing the PIC and quencher and subsequent storage of the pathogen inactivated RBCs (see e.g., FIGS. 18, 19). Such processing sets may comprise an openable seal (e.g., clamp, frangible seal) to restrict (e.g., prevent) fluid transfer between a container used for incubation of the blood component mixed with PIC (and quencher) and the additive solution container until desired by the user. In some embodiments, the container containing additive solution for replacing the PIC (and quencher) and subsequent storage of the pathogen inactivated RBCs is separate from (e.g., not part of) the processing set and may be used in conjunction with the processing set, such as for example by sterile “welding” to the processing set after incubation of the RBCs with PIC and quencher (see e.g., FIG. 21).

In one exemplary process for preparing a pathogen inactivated RBC blood composition with a system and methods of the present application (see, e.g., FIG. 22), a a reusable consumable portion of the system, comprising 1) connectors (e.g., input connectors) and fluid paths (e.g., tubing) for transferring each of a quencher (e.g., GSH) and processing solution (e.g., from respective multi-use containers) to a manifold, and associated pump, and a fluid path from the manifold to a connector (e.g., output connector) for coupling a disposable processing set, and 2) connectors (e.g., input connectors) and fluid paths (e.g., tubing) for transferring each of a PIC (e.g., S-303) and diluent (e.g., saline) (e.g., from respective multi-use containers) to a manifold, and associated pump, 3) a container for diluting the PIC with diluent connected (e.g., by way of a fluid path such as tubing) to the manifold (e.g., PIC fluid path manifold), and a fluid path from the manifold(s) to a connector (e.g., output connector) for coupling a disposable processing set, is loaded to the device by an operator. PIC and quencher are each reconstituted with saline in multi-dose bulk containers (e.g., dual chamber bags), with the PIC reconstituted at higher concentrations (e.g., 75 mM) than will ultimately be transferred after further dilution. In some embodiments, the bulk containers of GSH and PIC are reconstituted at a desired concentration in a sufficient volume for dosing at least 6 or more, 12 or more, 18 or more, or 24 or more RBC units in disposable processing sets. Multi-dose bulk containers of reconstituted PIC, reconstituted quencher, processing solution and diluent are then reversibly coupled to the consumable portion of the system by way of one or more connector types (e.g., Luer connector, CSTD connector). In some preferred embodiments a connector for coupling the PIC container is a CSTD connector, such as for example, when a PIC is classified as a highly potent compound that requires a higher level of containment. RBCs are transferred from an RBC blood component container to the processing set (e.g., into a mixing container of the processing set) and the RBC component container is subsequently disconnected (e.g., by heat sealing tubing) from the processing set after transfer of the RBCs. The processing set is coupled (e.g., prior to or after addition of RBCs) to the consumable portion of the system, and the quencher (e.g., a determined amount of quencher based on RBC volume) and processing solution are then transferred to the processing set (e.g., into a mixing container) containing the RBCs. The PIC and diluent are transferred into the consumable portion of the system (e.g., concurrently or after the quencher and processing solution transfer) and the PIC diluted to a desired concentration with the diluent. The diluted PIC is then transferred to the processing set (e.g., mixing container) already containing the RBCs, processing solution and quencher, and the processing set containing the RBCs, PIC, quencher and processing solution is then disconnected from the consumable portion of the system for incubation (e.g., in the mixing container or another container of the processing set). In some embodiments, the mixture of RBCs, PIC, quencher and processing solution is transferred from the mixing container to a second container configured for incubating the mixture (e.g., incubation container). Following incubation, PIC, quencher and processing solution are separated from the RBCs (e.g., by centrifugation) and replaced with an additive solution (e.g., RBC additive solution). In some preferred embodiments, the additive solution is provided in a separate container that is not connected to (e.g., not part of) the processing set, such container configured for storage of the pathogen inactivated RBCs (e.g., storage container), and the storage container is connected to an incubation container of the processing set by heat welding (e.g., sterile welding, tubing welding). After replacing the replacement with additive solution, the pathogen-inactivated RBCs in additive solution are stored in the storage container (e.g., after disconnecting the storage container by heat sealing of tubing). Providing the additive solution in a separate container that is not connected to (e.g., part of) the processing set may provide certain advantages. For example, processing sets that comprise only “dry” components and no “wet” components (e.g., fluid containing), provide for more simplified manufacturing and sterilization, and decreased cost of goods (e.g., “dry” processing sets can be irradiated for sterilization). A “wet” container with additive solution may be steam sterilized. Additionally, a separate storage container with additive solution provides an opportunity to supply different additive solutions based on different geographic markets (e.g., different solutions approved in different countries), without the need for multiple processing set configurations by geography. Additional advantages may include no requirement for transfer of additive solution through the system (e.g., fewer components, fewer connectors).

In other embodiments, two or more dosing devices as described above may be used for higher throughput processing (pathogen inactivation) of RBC units. In one such method, one device is used for dosing quencher (e.g., GSH) to a processing set and a separate device is used for dosing the pathogen inactivation compound (e.g., S-303). For example, a bulk container of GSH is reconstituted in water (e.g., in a dual chamber bag or syringe) at a sufficient volume to dose 6 or more, 12 or more, 18 or more, or 24 or more RBC units in disposable processing sets, by reversibly (and sterilely) coupling the GSH container to a fluid path (e.g., sterile tubing) at the device, coupling the first processing set to a fluid path (e.g., sterile tubing) at the device, transferring 15 mL of the reconstituted GSH with accuracy of at least 2% into the mixing container of the processing set, which mixing container already contains processing solution and admixing, transferring the RBCs into the mixing container, uncoupling the processing set (before or after transferring the RBCs), and repeating with the next processing set, up to any maximum number of processing sets for the bulk container of GSH. A bulk container of S-303 is reconstituted in saline or water (e.g., in a dual chamber bag or syringe), such as for example at 50 mM, in a sufficient volume to dose 6 or more, 12 or more, 18 or more, or 24 or more RBC units in the disposable processing sets by reversibly (and sterilely) coupling the S-303 container to a fluid path (e.g., sterile tubing) at a second device, coupling the mixing container of the uncoupled processing set containing GSH and RBCs (from above) to a fluid path (e.g., sterile tubing) at a second device, diluting a portion of the S-303 (e.g., 2.22 mL) to 6 mM with saline at an accuracy within 2%, transferring 18.5 mL of the diluted S-303 with an accuracy of at least 2% to the mixing container of the processing set that contains the quencher and RBCs, uncoupling the processing set, and repeating with the next processing set to which GSH and RBCs have already been added. Such methods provide a means for high throughput pathogen inactivation of larger number of RBC that otherwise is not readily achievable with current single-use processing sets.

### RBCs

In some embodiments, a pathogen inactivation process of the present disclosure may comprise adding (e.g., transferring) a quencher to a pathogen inactivation processing set (e.g., to a mixing bag of a processing set). The addition of quencher, such as for example, from a bulk container of quencher (e.g., quencher solution), may occur before or after the blood component (e.g., RBC) to be treated is added to the processing set (e.g., mixing bag of processing set). In some embodiments, a quencher is mixed with a processing solution in the processing set (e.g., in a mixing bag of processing set). In some embodiments, a specified (e.g., determined) amount (e.g., amount calculated based on RBC volume) of quencher is added to the processing set (e.g., mixing bag of processing set) using a dosing device of the present disclosure, wherein the processing set contains the processing solution prior to coupling the mixing bag or processing set comprising the mixing bag to the dosing device system (e.g., processing set manufactured with processing solution). n some embodiments, the processing solution is transferred from a processing solution container to the processing set by the dosing device system prior. In some embodiments, a quencher may be mixed with the processing solution by the dosing device system prior to transfer to the processing set. In some embodiments, a specified amount (e.g., amount calculated based on RBC volume) of quencher is added to the processing set (e.g., mixing bag of processing set) using a dosing device of the present disclosure.

All references, including publications, patent applications, and patents, cited herein are hereby incorporated by reference to the same extent as if each reference were individually and specifically indicated to be incorporated by reference and were set forth in its entirety herein.

1. A method of preparing a pathogen inactivated blood composition, comprising:
at a device comprising one or more components configured to control flow of one or more fluids:
(a) coupling a container containing a pathogen inactivation compound (PIC; PIC container) to a first fluid path, optionally wherein the first fluid path is loaded to the device prior to coupling the PIC container to the first fluid path;
(b) coupling a processing set to the first fluid path;
(c) transferring a blood composition from a container containing the blood composition (blood composition container) into a container of the processing set configured for mixing the blood composition with the PIC (mixing container);
(d) determining an amount of the PIC to pathogen-inactivate the blood composition based at least in part on one or more input parameters; and
(e) transferring the determined amount of the PIC from the PIC container into the mixing container via the first fluid path using the one or more components configured to control flow of fluids.

2-3. (canceled)

4. The method of claim 1, wherein the amount of the PIC to pathogen-inactivate the blood composition is determined based at least in part on weight and/or volume of the blood composition; optionally wherein the amount of the PIC to pathogen-inactivate the blood composition is determined based at least in part by calculating the volume of the blood composition based on the weight of the blood composition.

5-6. (canceled)

7. The method of claim 1, wherein the device further comprises one or more components configured to receive an input, and determining the amount of the PIC to pathogen-inactivate the blood composition further comprises receiving an input indicating the weight of the blood composition; optionally wherein the input further indicates the determined amount of the PIC.

8-9. (canceled)

10. The method of claim 7, wherein the device further comprises a scale, and the received input comprises data representing the weight of the blood composition from the scale; and/or wherein the one or more components configured to receive an input comprise a touchscreen display or keyboard, and the received input is a user input from the touchscreen display or keyboard.

11. The method of claim 1, wherein the one or more input parameters comprise one or more of: a type of the blood composition, a volume of the blood composition, a number of platelets or red blood cells in the blood composition, a type of solution in the blood composition, a type of PIC, a target PIC concentration, and a weight of the blood composition.

12-16. (canceled)

17. The method of claim 1, further comprising terminating fluid transfer from the PIC container into the mixing container via the first fluid path using one or more of the components configured to control flow of fluids based at least in part on detecting transfer of the determined amount of the PIC into the mixing container.

18. (canceled)

19. The method of claim 1, wherein the device further comprises a scale, and the method further comprises weighing the mixing container before, during, and/or after transfer of the determined amount of the PIC into the mixing container; optionally wherein the method further comprises: terminating fluid transfer from the PIC container into the mixing container via the first fluid path using one or more of the components configured to control flow of fluids based at least in part on detecting transfer of a weight corresponding to the determined amount of the PIC into the mixing container using the scale.

20. The method of claim 1, wherein the first fluid path comprises sterile tubing; optionally wherein the first fluid path further comprises one or more of: a connector, a breakable connector, a cannula, a filter, a manifold, a pump, a container, and a coupling.

21. The method of claim 1, further comprising coupling a container containing a diluent (diluent container) to the first fluid path.

22. The method of claim 21, wherein transferring the determined amount of the PIC from the PIC container into the mixing container via the first fluid path comprises:
(i) transferring diluent from the diluent container into a third container, wherein the first fluid path further comprises the third container;
(ii) transferring PIC from the PIC container into the third container; and
(iii) transferring diluent and the determined amount of the PIC from the third container into the mixing container.

23. (canceled)

24. The method of claim 21, wherein the PIC in the PIC container is at a concentration at least about 2 times greater than the concentration of the determined amount of PIC transferred into the mixing container; optionally wherein the PIC in the PIC container is at a concentration about 2 times to about 50 times greater than the concentration of the determined amount of PIC transferred into the mixing container.

25. The method of claim 21, wherein the determined amount of the PIC is transferred into the mixing container at a volume accuracy of within about 2% of the determined amount.

26. (canceled)

27. The method of claim 1, wherein the device is not an automated separation device.

28. The method of claim 1, wherein the processing set comprises:
(i) the mixing container, within which the blood composition in admixture with the determined amount of the PIC can be subjected to ultraviolet light; and
(ii) at least a first storage container, coupled to the mixing container, wherein the first storage container is configured for storing the pathogen-inactivated blood composition; wherein optionally the processing set comprises one or more filters.

29. The method of claim 1, wherein the blood composition comprises a platelet and/or a plasma composition.

30. The method of claim 29, wherein after (e) the PIC is at a concentration of about 40 μM and about 70 μM in the mixing container with the blood composition; optionally wherein after (e) the PIC is at a concentration of about 50 μM and about 60 μM, optionally about 55 μM, in the mixing container with the blood composition.

31-34. (canceled)

35. The method of claim 29, further comprising calculating a light dose for subjecting the blood composition and PIC to ultraviolet light to photochemically inactivate a pathogen(s) if present in the blood composition using the determined amount of the PIC based at least in part on one or more of: a type of the blood composition, a volume of the blood composition, a type of PIC, a concentration of the PIC, a volume of the container for illumination, a number of platelets in the blood composition, a type of solution in the blood composition, and a weight of the blood composition.

36. (canceled)

37. The method of claim 35, further comprising transferring data indicating the calculated light dose to a second device or server; optionally wherein the method further comprises transferring to a second device or server data to calculate a light dose for photochemical inactivation of the blood composition using the determined amount of the PIC; optionally wherein the second device is an illumination device.

38. The method of claim 29, wherein the PIC is a photoactive pathogen inactivating compound selected from the group consisting of a psoralen, an isoalloxazine, an alloxazine, a phthalocyanine, a phenothiazine, a porphyrin, and merocyanine 540.

39-40. (canceled)

41. The method of claim 1, wherein the processing set comprises:
(i) the mixing container, which is configured for mixing the blood composition and the determined amount of the PIC; and
(ii) a second container, coupled to the mixing container, wherein the second container is configured for incubating the blood composition and the determined amount of the PIC; wherein optionally the processing set comprises one or more filters.

42. The method of claim 41, wherein the processing set further comprises: (iii) at least a first storage container, coupled to or configured to be coupled to the second container, wherein the first storage container is configured for storing the pathogen-inactivated blood composition.

43-44. (canceled)

45. The method of a claim 41, wherein the blood composition comprises red blood cells (RBCs).

46. The method of claim 45, further comprising mixing the blood composition with a quencher.

47. (canceled)

48. The method of claim 46, further comprising:
(i) coupling a quencher container to a second fluid path; and
(ii) transferring quencher from the quencher container into the mixing container via the second fluid path using one or more of the components configured to control flow of fluids.

49. (canceled)

50. The method of claim 48, further comprising transferring a processing solution from a processing solution container into the mixing container using one or more of the components configured to control flow of fluids at the second device.

51-54. (canceled)

55. The method of claim 45, wherein the PIC comprises a nucleic acid binding ligand that is an intercalator, optionally wherein the intercalator is an acridine; optionally wherein the PIC is β-alanine, N-(acridin-9-yl), 2-[bis(2-chloroethyl)amino]ethyl ester.

56. The method of claim 46, wherein the quencher comprises cysteine or a derivative of cysteine; optionally wherein the quencher is a peptide of 3-6 amino acids, wherein at least one of the amino acids is cysteine, N-acetyl cysteine, or S-acetyl cysteine.

57-58. (canceled)

59. The method of claim 46, wherein the quencher is transferred into the mixing container at a volume accuracy of within about 2% of the determined amount.

60. The method of claim 1, wherein the first fluid path is configured for multiple uses; optionally wherein the first fluid path is part of a reusable consumable configured for use in a dosing device system, wherein the reusable consumable comprises one or more fluid path(s), one or more connector(s), one or more manifold(s), and optionally one or more pump(s).

61. The method of claim 1, wherein the PIC container, the processing set, and/or the mixing container are reversibly coupled to the first fluid path.

62-66. (canceled)

67. A system for preparing a pathogen-inactivated blood composition, comprising:
(a) a first fluid path configured to be removably mounted to a device for controlling flow of one or more fluids, wherein the first fluid path is configured to be coupled to a container containing a pathogen inactivation compound (PIC), and configured to be coupled to a processing set, and wherein the first fluid path is configured for transfer of the PIC to the processing set;
(b) a device comprising one or more components configured to control flow of one or more fluids through the first fluid path; and
(c) the processing set, comprising:
(i) a container configured for mixing a blood composition with the PIC (mixing container), wherein the mixing container is configured to be coupled to the first fluid path; and at least a first storage container, coupled to the mixing container, wherein the storage container is configured for storing the pathogen-inactivated blood composition; or
(ii) a container configured for mixing a blood composition with a PIC (mixing container), wherein the mixing container is configured to be coupled to the first fluid path; a second container, coupled to the mixing container, wherein the second container is configured for incubating the blood composition in admixture with the PIC and the second container is configured to be connected to at least a first storage container configured for storing the pathogen-inactivated blood composition; or
(iii) a container configured for mixing a blood composition with a PIC (mixing container), wherein the mixing container is configured to be coupled to the first fluid path; a second container, coupled to the mixing container, wherein the second container is configured for incubating the blood composition and the PIC; and at least a first storage container, coupled to the second container, wherein the storage container is configured for storing the pathogen-inactivated blood composition; or
(iv) a container configured for mixing a blood composition with a PIC (mixing container), wherein the mixing container is configured to be coupled to the first fluid path; a second container, coupled to the mixing container, wherein the second container contains a compound adsorption device (CAD); and at least a first storage container, coupled to the second container, wherein the storage container is configured for storing the pathogen-inactivated blood composition.

68. (canceled)

69. The system of claim 67, wherein the first fluid path is part of a reusable consumable of the system, wherein the reusable consumable comprises one or more fluid paths, one or more connectors, one or more manifolds, and optionally one or more pumps; optionally wherein the first fluid path comprises a manifold and/or a pump.

70. The system of claim 67, wherein the mixing container is configured for subjecting a blood composition and PIC contained therein to ultraviolet light.

71. The system of claim 67, further comprising a second fluid path configured to be removably mounted to a device for controlling flow of one or more fluids, wherein the second fluid path is configured to be coupled to a container containing a quencher, and configured to be coupled to the processing set, and wherein the second fluid path is configured for transfer of quencher to the processing set; optionally, wherein the second fluid path is part of a reusable consumable of the system, wherein the reusable consumable comprises one or more fluid paths, one or more connectors, one or more manifolds, and optionally one or more pumps; optionally, wherein the second fluid path comprises a manifold and/or a pump.

72. The system of claim 71, further comprising a third fluid path configured to be removably mounted to a device for controlling flow of one or more fluids, wherein the third fluid path is configured to be coupled to a container containing a processing solution, and wherein the third fluid path is connected to a manifold or configured to be coupled to the processing set, and wherein the third fluid path is configured for transfer of processing solution to the processing set.

73. The system of claim 67, further comprising a fourth fluid path configured to be removably mounted to a device for controlling flow of one or more fluids, wherein the fourth fluid path is configured to be coupled to a container containing a diluent solution, and wherein the fourth fluid path is connected to a manifold, and wherein the manifold is configured for mixing the PIC and the diluent.

74. (canceled)

75. The system of claim 67, wherein the device further comprises one or more components configured to receive an input; optionally, wherein the input comprises one or more of a type of the blood composition, a volume of the blood composition, a number of platelets or red blood cells in the blood composition, a type of solution in the blood composition, a type of PIC, a target concentration of PIC, and a weight of the blood composition.

76. (canceled)

77. The system of claim 67, wherein the device further comprises a scale configured to weigh a blood composition and/or the mixing container.

78. The system of claim 67, wherein the device further comprises one or more processors configured to determine an amount of the PIC to add to the blood composition to pathogen-inactivate the blood composition; optionally, wherein the amount of the PIC is determined based at least in part on weight and/or volume of the blood composition; optionally, wherein the amount of the PIC is determined based at least in part by calculating the volume of the blood composition based on the weight of the blood composition.

79. (canceled)

80. The system of claim 67, wherein the device is configured to calculate a light dose for subjecting the blood composition and PIC to ultraviolet light to photochemically inactivate a pathogen(s) if present in the blood composition using the PIC based at least in part on one or more of: a type of the blood composition, a volume of the blood composition, a type of PIC, a concentration of the PIC, a volume of the container for illumination, a number of platelets in the blood composition, a type of solution in the blood composition, and a weight of the blood composition.

81. (canceled)

82. The system of claim 80, wherein the device is configured to transfer data indicating the calculated light dose to a second device or server; optionally wherein the device is configured to transfer to a second device or server data to calculate a light dose for subjecting the blood composition and PIC to ultraviolet light to photochemically inactivate a pathogen(s) if present in the blood composition using the PIC; optionally wherein the second device is an illumination device.

83. (canceled)

84. The system of claim 67, wherein the system is configured to terminate fluid transfer from the PIC container into the mixing container via the first fluid path using one or more of the components configured to control flow of fluids based at least in part on detecting transfer of the determined amount of the PIC into the mixing container; optionally, based at least in part on detecting transfer of the determined amount of the PIC through the first fluid path using the one or more fluid detectors.

85. The system of claim 67, wherein the system and/or the fluid path comprises one or more of: a connector, a breakable connector, a cannula, a filter, a manifold, a pump, a container, and a coupling.

86. The system of claim 67, wherein the device comprises a scale, and the system is configured to weigh the mixing container before, during, and/or after transfer of the determined amount of the PIC into the mixing container; optionally, wherein the system is configured to terminate fluid transfer from the PIC container into the mixing container via the using one or more of the components configured to control flow of fluids based at least in part on detecting transfer of a weight corresponding to the determined amount of the PIC into the mixing container using the scale.

87. The system of claim 67, wherein the system is configured to transfer a determined amount of PIC to a processing set at a volume accuracy of within about 2% of the determined amount.

88-89. (canceled)

90. The system of claim 67, wherein the system is configured to calculate a light dose for subjecting the blood composition and PIC to ultraviolet light to photochemically inactivate a pathogen(s) if present in a blood composition using a determined amount of the PIC based at least in part on one or more of: a type of the blood composition, a volume of the blood composition, a type of PIC, a concentration of the PIC, a volume of the container for illumination, a number of platelets in the blood composition, a type of solution in the blood composition, and a weight of the blood composition.

91. (canceled)

92. The system of claim 67, wherein the device is not an automated separation device.

93. The system of claim 67, wherein the processing set is configured to be reversibly coupled to one or more fluid paths, optionally, wherein the processing set comprises a fluid path configured for coupling (e.g., via connector(s)) to a first fluid path from a PIC container and/or a second fluid path from a quencher container; and/or wherein the processing set comprises a fluid path configured to be connected to a blood composition container; and/or wherein the processing set comprises one or more filters.

94-96. (canceled)
